(19) United States
(12) Patent Application Publication
(10) Pub. No.: US 2023/0189859 A1
(43) Pub. Date: Jun. 22, 2023

(54) COMPOSITIONS COMPRISING COOLNESS-REGULATING AGENT, AND METHOD OF USE THEREOF

(71) Applicant: EPC Natural Products Co., Ltd., Beijing (CN)

(72) Inventors: Jingang SHI, Beijing (CN); Hansheng WANG, Beijing (CN); Xiaorui ZHANG, Beijing (CN); Weiyao SHI, Beijing (CN); Thomas Eidenberger, Wels (AT)

(21) Appl. No.: 18/060,894

(22) Filed: Dec. 1, 2022

Related U.S. Application Data
(63) Continuation of application No. PCT/CN2022/135489, filed on Nov. 30, 2022.
(60) Provisional application No. 63/264,895, filed on Dec. 3, 2021.

(51) Int. Cl. A23L 27/30; A23G 3/42; A23L 2/60; A23L 2/56; A61K 8/60; A61Q 11/00

(52) U.S. Cl. CPC A23L 27/33; A23G 3/42; A23L 2/56; A23L 2/60; A61K 8/602; A61Q 11/00

(57) ABSTRACT

Compositions comprising one or more natural high intensity sweeteners and/or derivatives thereof are disclosed. The one or more high intensity sweeteners and/or derivatives regulate the coolness taste or flavor of the composition.

This application claims priority from U.S. Provisional Pat. Application No. 62/264,895, filed Dec. 3, 2021 and PCT Applicstion No. PCT/CN2022/135489, filed Nov. 30, 2022, both of which are incorporated herein by reference.

### FIELD

The present disclosure relates generally to a composition containing one or more agent(s) providing or enhancing a cooling effect or taste and the use of such composition in consumable products.

### BACKGROUND

High intensity sweeteners (HIS) have been widely used in the food and beverage products as sugar replacement. Presently, the sensory benchmarks associated with products containing HIS, including the natural HIS (NHIS), such as steviol glycosides, sweet tea extracts and monk fruit extracts, are mostly predicated on the taste of sugar sweetness. There is need for further exploring the characteristics of natural HIS sweeteners and their use in the food and beverage products.

### SUMMARY

One aspect of the present application relates to a composition comprising an agent that regulates a cooling effect or taste, wherein the agent comprises one or more nature high-intensity sweeteners (NHIS) and/or derivatives of NHIS, and wherein the agent is present in the composition in a sufficient amount to regulate a cooling effect or taste.

Another aspect of the present application relates to a method of improving a taste profile of a consumable product. The method comprises the step of adding to the consumable product, a sufficient amount of the composition of the present application, wherein the coolness of the consumable product is improved after the addition of the composition.

Another aspect of the present application relates to a method to regulate a salty taste of a salt-containg consumable. The method comprises the step of adding to the salt-containing consumable a composition comprising a Maillard reaction product, wherein the Maillard reaction product is prepared from a reaction mixture that comprise a glycosylated steviol glycoside, and wherein the Maillard reaction product is added at a final concentration of 1 ppm to 10,000 ppm.

Another aspect of the present application relates to a flavor or sweetening composition. The flavor or sweetening composition comprises one or more high intensity sweeteners and glycerin, wherein the weight ratio of glycerin-to-high intensity sweeteners is in the range of 99:1 to 1:99.

Another aspect of the present application relates to a food or beverage. The food or beverage comprises one or more high intensity sweeteners and glycerin, wherein the glycerin is present in the food of beverage at a concentration of 1 ppm to 10,000 ppm.

One of ordinary skill will understand that the differing embodiments disclosed in this application can all be used either independently or in combination with each other and there is no limitation implied on such combinations by the order or manner in which embodiments are disclosed.

### BRIEF DESCRIPTION OF DRAWINGS

While the present disclosure will now be described in detail, and it is done so in connection with the illustrative embodiments, it is not limited by the particular embodiments illustrated in the figures and the appended numbered paragraphs.

FIG. 1, Panel A shows relative cooling strengths of product 18-01 to 18-12 in Example 18. Panel B shows cooling longevity of product 18-01 to 18-06 (methol samples without added sugar). Panel C shows cooling longevity of product 18-07 to 18-12 (methol samples with added sugar).

Panel FIG. 2 shows the excitatory action of different concentration GRU90-MRP-FTA (product 2-02 in Example 2) sample on TRPM8 channel current in S6-211013001 cell. Panel A is a current-time diagram showing the current value changes during the whole recording time, which is related to the samples adding order. Panel B is a current representative diagram showing the current generated from a voltage stimulation.

FIG. 3 shows the excitatory action of different concentration GRU90-MRP-FTA (product 2-02 in Example 2) sample on TRPM8 channel current in S6-211013002 cell. Panel A is a current-time diagram showing the current value changes during the whole recording time, which is related to the samples adding order. Panel B is a current representative diagram showing the current generated from a voltage stimulation.

FIG. 4 shows the excitatory action of different concentration GRU90-MRP-FTA (product 2-02 in Example 2) sample on TRPM8 channel current in S6-211013003 cell. Panel A is a current-time diagram showing the current value changes during the whole recording time, which is related to the samples adding order. Panel B is a current representative diagram showing the current generated from a voltage stimulation.

FIG. 5 shows the excitatory action of different concentration GRU90-MRP-FTA (product 2-02 in Example 2) sample on TRPM8 channel current in S6-211013004 cell. Panel A is a current-time diagram showing the current value changes during the whole recording time, which is related to the samples adding order. Panel B is a current representative diagram showing the current generated from a voltage stimulation.

FIG. 6 shows the regulating action of different concentration GRU90-MRP-FTA (product 2-02 in Example 2) sample on TRPM8 channel current in S6-211013002 cell Panel A is a current-time diagram showing the current value changes during the whole recording time, which is related to the samples adding order. Panel B is a current representative diagram showing the current generated from a voltage stimulation.

FIG. 7 shows the regulating action of different concentration GRU90-MRP-FTA (product 2-02 in Example 2) sample on TRPM8 channel current in S6-211013003 cell. Panel A is a current-time diagram showing the current value changes during the whole recording time, which is related to the samples adding order. Panel B is a current representative diagram showing the current generated from a voltage stimulation.

FIG. 8 shows the regulating action of different concentration GRU90-MRP-FTA (product 2-02 in Example 2) sample on TRPM8 channel current in S6-211013004 cell. Panel A is a current-time diagram showing the current value changes during the whole recording time, which is related to the samples adding order. Panel B is a current representative diagram showing the current generated from a voltage stimulation.

FIG. 9 shows the excitatory action of different concentration GRU90-MRP-FTA (product 2-02 in Example 2) sample on TRPM4 channel current in S6-211208007 cell. Panel A is a current-time diagram showing the current value changes during the whole recording time, which is related to the samples adding order. Panel B is a current representative diagram showing the current generated from a voltage stimulation.

FIG. 10 shows the excitatory action of different concentration GRU90-MRP-FTA (product 2-02 in Example 2) sample on TRPM4 channel current in S6-211208010 cell. Panel A is a current-time diagram showing the current value changes during the whole recording time, which is related to the samples adding order. Panel B is a current representative diagram showing the current generated from a voltage stimulation.

FIG. 11 shows the regulating action of different concentration GRU90-MRP-FTA (product 2-02 in Example 2) sample on TRPM4 channel current in S6-211208008 cell. Panel A is a current-time diagram showing the current value changes during the whole recording time, which is related to the samples adding order. Panel B is a current representative diagram showing the current generated from a voltage stimulation.

FIG. 12 shows the regulating action of different concentration GRU90-MRP-FTA (product 2-02 in Example 2) sample on TRPM4 channel current in S6-211208010 cell. Panel A is a current-time diagram showing the current value changes during the whole recording time, which is related to the samples adding order. Panel B is a current representative diagram showing the current generated from a voltage stimulation.

FIG. 13 shows the excitatory action of different concentration GRU90-MRP-FTA (product 2-02 in Example 2) sample on TRPV3 channel current in S6-211208003 cell. Panel A is a current-time diagram showing the current value changes during the whole recording time, which is related to the samples adding order. Panel B is a current representative diagram showing the current generated from a voltage stimulation.

FIG. 14 shows the excitatory action of different concentration GRU90-MRP-FTA (product 2-02 in Example 2) sample on TRPV3 channel current in S6-211208004 cell. Panel A is a current-time diagram showing the current value changes during the whole recording time, which is related to the samples adding order. Panel B is a current representative diagram showing the current generated from a voltage stimulation.

FIG. 15 shows the regulating action of different concentration GRU90-MRP-FTA (product 2-02 in Example 2) sample on TRPV3 channel current in S6-211208003 cell. Panel A is a current-time diagram showing the current value changes during the whole recording time, which is related to the samples adding order. Panel B is a current representative diagram showing the current generated from a voltage stimulation.

FIG. 16 shows the regulating action of different concentration GRU90-MRP-FTA (product 2-02 in Example 2) sample on TRPV3 channel current in S6-211208003 cell. Panel A is a current-time diagram showing the current value changes during the whole recording time, which is related to the samples adding order. Panel B is a current representative diagram showing the current generated from a voltage stimulation.

One of ordinary skill will understand that the differing embodiments disclosed in this application can all be used either independently or in combination with each other and there is no limitation implied on such combinations by the order or manner in which embodiments are disclosed.

### DETAILED DESCRIPTION

### I. Definitions

Unless defined otherwise, all technical and scientific terms used herein have the same meanings as commonly understood by one of ordinary skill in the art to which this application belongs. All publications and patents specifically mentioned herein are incorporated by reference in their entirety for all purposes including describing and disclosing the chemicals, instruments, statistical analyses and methodologies which are reported in the publications which might be used in connection with the application. All references cited in this specification are to be taken as indicative of the level of skill in the art. Nothing herein is to be construed as an admission that the application is not entitled to antedate such disclosure by virtue of prior invention.

In the specification and in the claims, the terms “including” and “comprising” are open-ended terms and should be interpreted to mean “including, but not limited to.... “ These terms encompass the more restrictive terms “consisting essentially of” and “consisting of.”

It must be noted that as used herein and in the appended claims, the singular forms “a,” “an,” and “the” include plural reference unless the context clearly dictates otherwise. Further, the terms “a” (or “an”), “one or more” and “at least one” can be used interchangeably herein. It is also to be noted that the terms “comprising,” “including,” “characterized by” and “having” can be used interchangeably. Further, any reactant concentrations described herein should be considered as being described on a weight to weight (w/w) basis, unless otherwise specified to the contrary (e.g., mole to mole, weight to volume (w/v), etc.).

As used herein, the term “glycoside” refers to a molecule in which a sugar (the “glycone” part or “glycone component” of the glycoside) is bonded to a non-sugar (the “aglycone” part or “aglycone component”) via a glycosidic bond.

The terms “steviol glycoside” and “SG” are used interchangeably with reference to a glycoside of steviol, a diterpene compound shown in Formula I, wherein one or more sugar residues are attached to the steviol compound of Formula I.

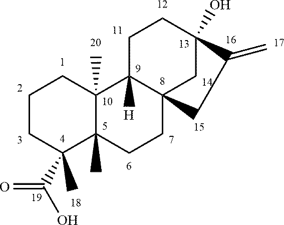

Steviol glycosides also include glycosides of isomers of steviol (isosteviol) as depicted in Formula II below, and derivatives of steviol, such as 12α-hydroxy-steviol and 15α-hydroxy-steviol.

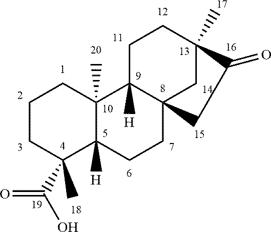

The terms “glycosidic bond” and “glycosidic linkage” refer to a type of chemical bond or linkage formed between the anomeric hydroxyl group of a saccharide or saccharide derivative (glycone) and the hydroxyl group of another saccharide or a non-saccharide organic compound (aglycone) such as an alcohol. The reducing end of the di- or polysaccharide lies towards the last anomeric carbon of the structure, whereas the terminal end lies in the opposite direction.

By way of example, a glycosidic bond in steviol and isosteviol involves the hydroxyl-group at the sugar carbon atom numbered 1 (so-called anomeric carbon atom) and a hydroxyl-group in the C19 carbonyl group of the steviol or isosteviol molecule building up a so-called O-glycoside or glycosidic ester. Additional glycosidic ester linkages can be formed at the hydroxyl group at C13 of steviol and at the carbonyl oxygen at C16 of isosteviol. Linkages at carbon atoms in the C1, C2, C3, C6, C7, C11, C12 and C15 positions of both steviol and isosteviol yield C-glycosides. In addition, C-glycosides can also be formed at the 2 methyl groups at C18 and C20 in both steviol and isosteviol.

The sugar part can be selected from any sugar with 3-7 carbon atoms, derived from either a dihydroxy-acetone (ketose) or a glycerin-aldehyde (aldose). The sugars can occur in open chain or in cyclic form, as D- or L- enantiomers and in α- or β-conformation.

Representative structures of possible sugar (Sug) conformations exemplified by glucose include D-glucopyranose and L-glucopyranose in which the position 1 is determinative of the α- or β-conformation:

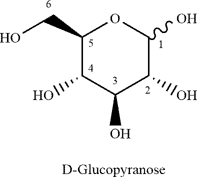

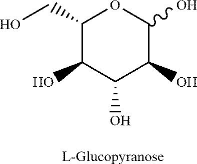

Pos 1: α- or β-conformation

The steviol glycosides for use in the sweetener or flavor composition of the present application include one or more glycosylated steviol glycoside (GSG) compounds with structures depicted in Table A.

TABLE A

Possible positions of sugar (Sug) molecules linked to steviol/isosteviol.

| Aglycone | Position | Sugar(Sug) | Conjugation | Category |
|---|---|---|---|---|
| Steviol | 13 | D- α D- β L- α L- β | D-Sug α (1-13) D-Sug β (1-13) L-Sug α (1-13) L-Sug β (1-13) | O-glucoside |
| Isosteviol | 16 | D- α/β L- α/β | D-Sug α/ β (1-16) L-Sug α/β (1-16) | O-glucoside (after reduction of keto-group) |
| Steviol Isosteviol | 19 | D/L- α/ β | D/L-Sug α/β (1-19) | Glucose-ester |
| Steviol | 1 | D/L- α/ β | D/L-Sug α/β (1-1) | C-glucoside |
| | 2 | | D/L-Sug α/β (1-2) | |
| | 3 | | D/L-Sug α/β (1-3) | |
| | (5) | | D/L-Sug α/β (1-5) | |
| | 6 | | D/L-Sug α/β (1-6) | |
| | 7 | | D/L-Sug α/β (1-7) | |
| | (9) | | D/L-Sug α/β (1-9) | |
| | 11 | | D/L-Sug α/β (1-11) | |
| | 12 | | D/L-Sug α/β (1-12) | |
| | 14 | | D/L-Sug α/β (1-14) | |
| | 15 | | D/L-Sug α/β (1-15) | |
| Steviol | (18) | D/L- α/ β | D/L-Sug α/β (1-18) | Methyl en-glucoside |
| | (20) | | D/L-Sug α/β (1-20) | |
| Isosteviol | 1 | D/L- α/ β | D/L-Sug α/β (1-1) | C-glucoside |
| | 2 | | D/L-Sug α/β (1-2) | |
| | 3 | | D/L-Sug α/β (1-3) | |
| | (5) | | D/L-Sug α/β (1-5) | |
| | 6 | | D/L-Sug α/β (1-6) | |
| | 7 | | D/L-Sug α/β (1-7) | |
| | (9) | | D/L-Sug α/β (1-9) | |
| | 11 | | D/L-Sug α/β (1-11) | |
| | 12 | | D/L-Sug α/β (1-12) | |
| | (13) | | D/L-Sug α/β (1-12) | |
| | 14 | | D/L-Sug α/β (1-14) | |
| | 15 | | D/L-Sug α/β (1-15) | |
| Isosteviol | (18) | D/L- α/ β | D/L-Sug α/β (1-18) | Methyl en-glucoside |
| | (20) | | D/L-Sug α/β (1-20) | |

Stevia plants contain a variety of different SGs in varying percentages. The phrase “steviol glycoside” is recognized in the art and is intended to include the major and minor constituents of Stevia. These “SGs” include, for example, stevioside, steviolbioside (SB), rebaudioside A (RA), rebaudioside B (RB), rebaudioside C (RC), rebaudioside D (RD), rebaudioside E (RE), rebaudioside F (RF), rebaudioside M (RM), rebaudioside O (RO), rebaudioside H (RH), rebaudioside I (RI), rebaudioside L (RL), rebaudioside N (RN), rebaudioside K (RK), rebaudioside J (RJ), rebaudioside U, rubusoside, dulcoside A (DA) as well as those listed in Tables A and B or mixtures thereof.

As used herein, the terms “rebaudioside A,” “Reb A,” “Reb-A” and “RA” are equivalent terms referring to the same molecule. The same condition applies to all lettered rebaudiosides with the exception of rebaudioside U, which may be referred to as Reb-U or Reb U, but not RU, so as to not be confused with rubusoside which is also referred to as RU.

Based on the type of sugar (i.e. glucose, rhamnose/deoxyhexose, xylose/arabinose) SGs can be grouped into three families (1) SGs with glucose; (2) SG with glucose and one rhamnose or deoxyhexose moiety; and (3) SGs with glucose and one xylose or arabinose moiety. The steviol glycosides for use in the present application are not limited by source or origin. Steviol glycosides may be extracted from Stevia leaves, synthesized by enzymatic processes, synthesized by chemical syntheses, or produced by fermentation.

Specific examples of steviol glycosides include, but are not limited to, the compounds listed in Table B-1 and isomers thereof. The steviol glycosides for use in the present application are not limited by source or origin. Steviol glycosides may be extracted from Stevia plants, Sweet tea leaves, synthesized by enzymatic processes or chemical syntheses, or produced by fermentation.

TABLE B-1

Exemplary steviol glycosides

| SG Name | MW | # Added Glucose moieties mw=180 | # Added Rhamnos e/Deoxy-hexose moieties mw=164 | # Added Xylose/ Arabinose moieties mw=150 | R1 (C-19) | R2 (C-13) | Backbone |
|---|---|---|---|---|---|---|---|
| Related SvGn# 1 | 457 | - | | | | | |
| Steviolmonoside | 479 | 1 | | | H- | Glcβ1- | Steviol |
| Steviolmonoside A | 479 | 1 | 1 | | Glcβ1- | H- | |
| SG-4 | 611 | 1 | | 1 | H- | Xy1β(1-2)Glcβ1- | Steviol |
| Dulcoside A1 | 625 | 1 | 1 | | H- | Rhaα(1-2)Glcβ1- | Steviol |
| Iso-steviolbioside | 641 | 2 | | | H- | Glcβ(1-2)Glcβ1- | Isosteviol |
| Reb-G 1 | 641 | 2 | | | H- | Glcβ(1-3)Glcβ1- | Steviol |
| Rubusoside | 641 | 2 | | | Glcβ1- | Glcβ1- | Steviol |
| Steviolbioside | 641 | 2 | | | H- | Glcβ(1-2)Glcβ1- | Steviol |
| Related SvGn#3 | 675 | - | | | | | |
| Reb-Fl | 773 | 2 | | 1 | H- | Xy1β(1-2)[Glcβ(1-3)]Glcβ1- | Steviol |
| Reb-Rl | 773 | 2 | | 1 | H- | Glcβ(1-2)[Glcβ(1-3)]Xylβ1- | Steviol |
| Stevioside F (SG-1) | 773 | 2 | | 1 | Glcβ1- | Xy1β(1-2)Glcβ1- | Steviol |
| SG-Unkl | 773 | 2 | | 1 | – | – | Steviol |
| Dulcoside A | 787 | 2 | 1 | | Glcβ1- | Rhaα(1-2)Glcβ1- | Steviol |
| Dulcoside B (JECFA C) | 787 | 2 | 1 | | H- | Rhaα(1-2)[Glcβ(1-3)]Glcβ1- | Steviol |
| SG-3 | 787 | 2 | 1 | | H- | 6-deoxyGlcβ(1-2)[Glcβ(1-3)]Glcβ1- | Steviol |
| Stevioside D | 787 | 2 | 1 | | Glcβ1- | Glcβ(1-2)6-deoxyGlcβ1- | |
| Iso-Reb B | 803 | 3 | | | H- | Glcβ(1-2)[Glcβ(1-3)]Glcβ1- | Isosteviol |
| Iso-Stevioside | 803 | 3 | | | Glcβ1- | Glcβ(1-2)Glcβ1- | Isosteviol |
| Reb B | 803 | 3 | | | H- | Glcβ(1-2)[Glcβ(1-3)]Glcβ1- | Steviol |
| Reb G | 803 | 3 | | | Glcβ1- | Glcβ(1-3)Glcβ1- | Steviol |
| Reb-KA | 803 | 3 | | | Glcβ(1-2)Glcβ1- | Glcβ1- | Steviol |
| SG-13 | 803 | 3 | | | Glcβ1- | Glcβ(1-2)Glcβ1- | Isomeric steviol (12α-hydroxy) |
| Stevioside | 803 | 3 | | | Glcβ1- | Glcβ(1-2)Glcβ1- | Steviol |
| Stevioside B (SG-15) | 803 | 3 | | | Glcβ(1-3)Glcβ1- | Glcβ1- | Steviol |
| Reb F | 935 | 3 | | 1 | Glcβ1- | Xylβ(1-2)[Glcβ(1-3)]Glcβ1- | Steviol |
| Reb R | 935 | 3 | | 1 | Glcβ1- | Glcβ(1-2)[Glcβ(1-3)]Xylβ1- | Steviol |
| SG-Unk2 | 935 | 3 | | 1 | – | – | Steviol |
| SG-Unk3 | 935 | 3 | | 1 | – | – | Steviol |
| Reb F3 (SG-11) | 935 | 3 | | 1 | Xylβ(1-6)Glcβ1- | Glcβ(1-2)Glcβ1- | Steviol |
| Reb F2 (SG-14) | 935 | 3 | | 1 | Glcβ1- | Glcβ(1-2)[Xylβ(1-3)]Glcβ1- | Steviol |
| Reb C | 949 | 3 | 1 | | Glcβ1- | Rhaα(1-2)[Glcβ(1-3)]Glcβ1- | Steviol |
| Reb C2/Reb S | 949 | 3 | 1 | | Rhaα(1-2)Glcβ1- | Glcβ(1-2)Glcβ1- | Steviol |
| Stevioside E (SG-9) | 949 | 3 | 1 | | Glcβ1- | 6-DeoxyGlcβ(1-2)[Glcβ(1-3)]Glcβ1- | Steviol |
| Stevioside E2 | 949 | 3 | 1 | | 6-DeoxyGlcpl- | Glcβ(1-2)[Glcβ(1-3)]Glcβ1- | |
| SG-10 | 949 | 3 | 1 | | Glcβ1- | Glcα(1-3)Glcβ(1-2)[Glcβ(1-3])Glcβ1- | Steviol |
| Reb L1 | 949 | 3 | 1 | | H- | Glcβ(1-3)Rhaα(1-2)[Glcβ(1-3)]Glcβ1- | Steviol |
| SG-2 | 949 | 3 | 1 | | Glcβ1- | 6-deoxyGlcβ(1-2)[Glcβ(1-3)]Glcβ1- | Steviol |
| Reb A3 (SG-8) | 965 | 4 (1 Fru) | | | Glcβ1- | Glcβ(1-2)[Fruβ(1-3)]Glcβ1- | |
| Iso-Reb A | 965 | 4 | | | Glcβ1- | Glcβ(1-2)[Glcβ(1-3)]Glcβ1- | Isosteviol |
| Reb A | 965 | 4 | | | Glcβ1- | Glcβ(1-2)[Glcβ(1-3)]Glcβ1- | Steviol |
| Reb A2 (SG-7) | 965 | 4 | | | Glcβ1- | Glcβ(1-6)[Glcβ(1-2) ]Glcβ1- | Steviol |
| Reb E | 965 | 4 | | | Glcβ(1-2)Glcβ1- | Glcβ(1-2)Glcβ1- | Steviol |
| Reb H1 | 965 | 4 | | | H- | Glcβ(1-6)Glcβ(1-3)[Glcβ1-3)]Glcβ1- | Steviol |
| Related SvGn#2 | 981 | | | | | | |
| Related SvGn#5 | 981 | | | | | | |
| Reb U2 | 1097 | 4 | | 1 | Xylβ(1-2)[Glcβ(1-3)]Glcβ1- | Glcβ(1-2)Glcβ1- | |
| Reb T | 1097 | 4 | | 1 | Xylβ(1-2)Glcβ1- | Glcβ(1-2)[Glcβ(1-3)]Glcβ1- | |
| Reb W | 1097 | 4 | | 1 | Glcβ(1-2)[Araβ(1-3)]Glcβ1- | Glcβ(1-2)Glcβ1- | |
| Reb W2 | 1097 | 4 | | 1 | Araβ(1-2)Glcβ1- | Glcβ(1-2)[Glcβ(1-3)]Glcβ1- | |
| Reb W3 | 1097 | 4 | | 1 | Araβ(1-6)Glcβ1- | Glcβ(1-2)[Glcβ(1-3)]Glcβ1- | |
| Reb U | 1097 | 4 | | 1 | Araα(1-2)-Glcβ1- | Glcβ(1-2)[Glcβ(1-3)]Glcβ1- | Steviol |
| SG-12 | 1111 | 4 | 1 | | Rhaα(1-2)Glcβ1- | Glcβ(1-2)[Glcβ(1-3)]Glcβ1- | Steviol |
| Reb H | 1111 | 4 | 1 | | Glcβ1- | Glcβ(1-3)Rhaα(1-2)[Glcβ(1-3)]Glcβ1- | Steviol |
| Reb J | 1111 | 4 | 1 | | Rhaα(1-2)Glcβ1- | Glcβ(1-2)[Glcβ(1-3)]Glcβ1- | Steviol |
| Reb K | 1111 | 4 | 1 | | Glcβ(1-2)Glcβ1- | Rhaα(1-2)[Glcβ(1-3)]Glcβ1- | Steviol |
| Reb K2 | 1111 | 4 | 1 | | Glcβ(1-6)Glcβ1- | Rhaα(1-2)[Glcβ(1-3)]Glcβ1- | Steviol |
| SG-Unk4 | 1111 | 4 | 1 | | – | – | Steviol |
| SG-Unk5 | 1111 | 4 | 1 | | – | – | Steviol |
| Reb D | 1127 | 5 | | | Glcβ(1-2)Glcβ1- | Glcβ(1-2)[Glcβ(1-3)]Glcβ1- | Steviol |
| Reb I | 1127 | 5 | | | Glcβ(1-3)Glcβ1- | Glcβ(1-2)[Glcβ(1-3)]Glcβ1- | Steviol |
| Reb L | 1127 | 5 | | | Glcβ1- | Glcβ(1-6)Glcβ(1-2)[Glcβ(1-3)]Glcβ1- | Steviol |
| Reb 13 | 1127 | 5 | | | [Glcβ(1-2)Glcβ( 1-6)]Glcβ1- | Glcβ(1-2)Glcβ1- | |
| SG-Unk6 | 1127 | 5 | | | – | – | Steviol |
| Reb Q (SG-5) | 1127 | 5 | | | Glcβ1- | Glcα(1-4)Glcβ(1-2)[Glcβ(1-3)]Glcβ1- | Steviol |
| Reb I2 (SG-6) | 1127 | 5 | | | Glcβ1- | Glcα(1-3)Glcβ1-2[Glcβ1-3)]Glcβ1- | Steviol |
| Reb Q2 | 1127 | 5 | | | Glcα(1-2)Glcα(1-4)Glcβ1- | Glcβ(1-2)Glcβ1- | |
| Reb Q3 | 1127 | 5 | | | Glcβ1- | Glcα(1-4)Glcβ(1-3)[Glcβ(1-2)]Glcβ1- | |
| Reb T1 | 1127 | 5 (1 Gal) | | | Galβ(1-2)Glcβ1- | Glcβ(1-2)[Glcβ(1-3)]Glcβ1- | |
| Related SvGn#4 | 1127 | | | | | | |
| Reb V2 | 1259 | 5 | | 1 | Xylβ(1-2)[Glcβ(1-3)]-Glcβ1- | Glcβ(1-2)[Glcβ(1-3)]Glcβ1- | Steviol |
| Reb V | 1259 | 5 | | 1 | Glcβ(1-2)[Glcβ(1-3)]Glcβ1- | Xylβ(1-2)[Glcβ(1-3)]-Glcβ1- | |
| Reb Y | 1259 | 5 | | 1 | Glcβ(1-2)[Araβ(1-3)]Glcβ1- | Glcβ(1-2)[Glcβ(1-3)]Glcβ1- | |
| Reb N | 1273 | 5 | 1 | | Rhaα(1-2)[Glcβ(1-3)]Glcβ1- | Glcβ(1-2)[Glcβ(1-3)]Glcβ1- | Steviol |
| Reb M | 1289 | 6 | | | Glcβ(1-2)[Glcβ(1-3)]Glcβ1- | Glcβ(1-2)[Glcβ(1-3)]Glcβ1- | Steviol |
| 15αOH Reb M | 1305 | 6 | | | Glcβ1-2(Glcβ1-3)Glcβ1- | Glcβ(1-2)[Glcβ1-3]Glcβ1- | 15α-Hydroxy-steviol |
| Reb O | 1435 | 6 | 1 | | Glcβ(1-3)Rhaα(1-2)[Glcβ(1-3)]Glcβ1- | Glcβ(1-2)[Glcβ(1-3)]Glcβ1- | Steviol |
| Reb O2 | 1435 | 6 | 1 | | Glcβ(1-4)Rhaα(1-2)[Glcβ(1-3)]Glcβ1- | Glcβ(1-2)[Glcβ(1-3)]Glcβ1- | |

Legend: SG-1 to 16: SGs without a specific name; SG-Unk1-6: SGs without detailed structural proof; Glc: Glucose; Rha: Rhamnose; Xyl: Xylose; Ara: Arabinose.

The terms “glycosylated steviol glycoside” and “GSG” refer to a molecule that (1) contains a SG backbone and one or more additional sugar residues, and (2) is artificially produced by glycosylation, conversion, fermentation or chemical synthesis, including isomers therefrom. For example, GRB contains a RB backbone and may be produced by glycosylation of RB or by alkaline hydrolysis of GRA.

The terms “non-Steviol glycoside”, “non-SG”, including glycosylated forms thereof, are used with reference to glycosides that are not present in Stevia plants or Stevia extracts. Exemplary non-Steviol glycosides or glycosylated forms thereof include, but are not limited to sweet tea extracts, swingle extracts, glycosylated sweet tea extracts, glycosylated swingle extracts, glycosylated sweet tea glycosides, glycosylated mogrosides, glycyrrhizin, glycosylated glycyrrhizin, rubusoside from sweet tea extract, glycosylated rubusoside from sweet tea extract, suaviosides, glycosylated suaviosides, mogrosides, glycosylated mogrosides and sucralose. The phrases “natural non-Steviol glycoside sweetener”, “natural non-SG sweetener”, including glycosylated forms thereof, are more broadly used with reference to non-Steviol glycosides, as well as other natural sweeteners that are not derived from Stevia plants or extracts, including but not limited to thaumatin, xylitol, monellin, brazzein, miraculin, curculin, pentadin, and mabinlin, and combination thereof. The phrase “non-Stevia sweetener” is more broadly used with reference to both natural non-SG sweeteners, as well as synthetic and semi-synthetic sweeteners as further described herein.

The terms “sweet tea extract” and “STE” refer to an extract prepared from the sweet tea (ST) plant. It should also be understood that an STE can be purified and/or separated into one or more sweet tea components (STCs).

The terms “sweet tea component” and “STC” refer to a component of an STE.

The terms “sweet tea glycoside” and “STG” refer to a glycoside derived from sweet tea plants or known to be present in sweet tea plants. Examples of STGs include, but are not limited to, rubusoside, suaviosides such as SU-A, SU-B, SU-C1, SU-D1, SU-D2, SU-E, SU-F, SU-G, SU-H, SU-I, and SU-J, steviolmonoside, rebaudioside A, 13-O-β-D-glucosyl-steviol, isomers of rebaudioside B, isomers of stevioside, panicloside IV and sugeroside. Some STGs, such as rubusoside, are also present in Stevia plants and are steviol glycosides (SGs).

The term “glycosylated sweet tea component (GSTC)” refers to a STC that has been subjected to an exogenously preformed glycosylation process. A GSTC may be artificially produced by enzymatic conversion, fermentation or chemical synthesis.

The term “glycosylated sweet tea glycoside (GSTG)” refers to a molecule that (1) contains a STG backbone and one or more additional sugar residues, and (2) is artificially produced by enzymatic conversion, fermentation or chemical synthesis.

The terms “glycosylated rubusoside” “glycosylated RU” and “GRU” are used interchangeably with reference molecules having a RU backbone with additional sugar units added in a glycosylation reaction under man-made conditions. GRUs include, but are not limited to, molecules having a RU backbone and 1-50 additional sugar units. As used herein, the term “sugar unit” refers to a monosaccharide unit.

As used herein, the term “enzymatically catalyzed method” refers to a method that is performed under the catalytic action of an enzyme, in particular of a glycosidase or a glycosyltransferase. The method can be performed in the presence of said glycosidase or glycosyltransferase in isolated (purified, enriched) or crude form.

The term “glycosyltransferase” (GT) refers to an enzyme that catalyzes the formation of a glycosidic linkage to form a glycoside. As used herein, the term “glycosyltransferase” also includes variants, mutants and enzymatically active portions of glycosyltransferases. Likewise, the term “glycosidase” also includes variants, mutants and enzymatically active portions of glycosidases.

The term “monosaccharide” as used herein refers to a single unit of a polyhydroxyaldehyde forming an intramolecular hemiacetal the structure of which including a six-membered ring of five carbon atoms and one oxygen atom. Monosaccharides may be present in different diasteromeric forms, such as α or β anomers, and D or L isomers. An “oligosaccharide” consists of short chains of covalently linked monosaccharide units. Oligosaccharides comprise disaccharides which include two monosaccharide units, as well as trisaccharides which include three monosaccharide units. A “polysaccharide” consists of long chains of covalently linked monosaccharide units.

The acronym “G-X” or “GX” refers to the glycosylation products of a composition “X”, i.e., product prepared from an enzymatically catalyzed glycosylation process with X and one or more sugar donors as the starting materials. For example, GSG refers to the glycosylation product of a steviol glycoside (SG).

As used herein, the term “Maillard reaction” refers to a non-enzymatic reaction of (1) one or more reducing and/or non-reducing sugars, and (2) one or more amine donors in the presence of heat, wherein the non-enzymatic reaction produces a Maillard reaction product and/or a flavor. Thus, this term is used unconventionally, since it accommodates the use of non-reducing sweetening agents as substrates, which were not heretofore thought to serve as substrates for the Maillard reaction.

The term “reaction mixture” refers to a composition comprising at least one amine donor and one sugar donor, wherein the reaction mixture is to be subjected to a Maillard reaction; a “reaction mixture” is not to be construed as the reaction contents after a Maillard reaction has been conducted, unless otherwise noted.

The term “sugar,” as used herein, refers to a sweet-tasting, soluble carbohydrate, typically used in consumer food and beverage products.

The term “sugar donor,” as used herein, refers to a sweet-tasting compound or substance from natural or synthetic sources, which can participate as a substrate in a Maillard reaction with an amine group-containing donor molecule.

The term “amine donor,” as used herein, refers to a compound or substance containing a free amino group, which can participate in a Maillard reaction.

The term “Maillard reaction product” or “MRP” refers to any compound produced by a Maillard reaction between an amine donor and a sugar donor in the form of a reducing sugar, non-reducing sugar, or both. Preferably, the sugar donor includes at least one carbonyl group. In certain embodiments, the MRP comprises a compound that provides a flavor (“Maillard flavor”), a color (“Maillard color”), or both.

As used hereinafter, the term “standard MRP” or “conventional MRP (C-MRP)” refers to an MRP formed from a reaction mixture that contains (1) at least one reducing sugar as sugar donor and (2) one or more free amino acids as amine donor.

The terms “Stevia-MRP” refers to the product of a Maillard reaction, wherein the starting material of the Maillard reaction comprises a Stevia extract (SE), a steviol glycoside (SG), a glycosylated Stevia extract (GSE), a glycosylated steviol glycoside (GSG) or combinations thereof. Accordingly, Stevia-MRPs include, but are not limited to, SE-MRPs, SG-MRPs, GSE-MRPs and GSG-MRPs.

The terms “MRP composition,” “Maillard product composition” and “Maillard flavor composition” are used interchangeably and refer to a composition comprising one or more MRPs, including SG-MRPs, SE-MRPs, GSG-MRPs, G-SE-MRPs, C-MRPs, etc.

The term “thaumatin”, as used herein, is used generically with reference to thaumatin I, II, III, a, b, c, etc. and/or combinations thereof.

The term “non-volatile”, as used herein, refers to a compound having a negligible vapor pressure at room temperature, and/or exhibits a vapor pressure of less than about 2 mm of mercury at 20° C.

The term “volatile”, as used herein, refers to a compound having a measurable vapor pressure at room temperature, and/or exhibits a vapor pressure of, or greater than, about 2 mm of mercury at 20° C.

As used herein, the term “sweetener” or “sweetening agent” generally refers to a consumable product, which produces a sweet taste when consumed alone. Examples of sweeteners include, but are not limited to, high-intensity sweeteners (HIS) and derivatives therof, bulk sweeteners, and low sweetness products produced by synthesis, fermentation or enzymatic conversion methods.

As used herein the term “high-intensity sweetener” or “HIS” refers to any synthetic sweetener, semi-synthetic sweetener, sweetener found in nature and derivatives thereof, that is sweeter than sucrose. High-intensity sweeteners are typically many times (e.g., 20 times and more, 30 times and more, 50 times and more or 100 times sweeter than sucrose). For example, sucralose is about 600 times sweeter than sucrose, sodium cyclamate is about 30 times sweeter, Aspartame is about 160-200 times sweeter, and thaumatin is about 2000 times sweeter then sucrose (the sweetness depends on the tested concentration compared with sucrose). High-intensity sweeteners are commonly used as sugar substitutes or sugar alternatives because they are many times sweeter than sugar but contribute only a few to no calories when added to foods. High-intensity sweeteners may also be used to regulate the flavor of foods. High-intensity sweeteners generally will not raise blood sugar levels.

As used herein, the term “natural high-intensity sweetener (NHIS)” refers to HIS found in nature, typically in plants, which may be in raw, extracted, purified, refined, or any other form, singularly or in combination thereof. A NHIS characteristically has higher sweetness potency, but fewer calories than sucrose, fructose, or glucose. Examples of NHIS include, but are not limited to, stevia extracts, sweet tea extracts, swingle extracts, licorice extracts, steviol glycosides, suaviosides, mogrosides, glycyrrhizin and salts thereof.

As used herein, the term “derivatives of NHIS,” includes but is not limited to, (1) glycosylation products of NHIS, (2) Maillard reaction products of NHIS, and (3) Maillard reaction products of the glycosylation products NHIS.

As used herein, the term “synthetic high intensity sweetener (SHIS)” or “artificial high intensity sweetener (AHIS)” refers to high intensity sweeteners that are not found in nature. Examples of SHIS include, but are not limited to, sucralose, aspartame, acesulfame-K (Ace-K), neotame, saccharin and aspartame, glycyrrhizic acid ammonium salt, sodium cyclamate, saccharin, advantame, neohesperidin dihydrochalcone (NHDC), salts and derivatives thereof.

As used herein, the term “bulk sweetener” refers to a sweetener, which typically adds both bulk and sweetness to a confectionery composition and includes, but is not limited to, sugars, sugar alcohols, sucrose, commonly referred to as “table sugar,” fructose, commonly referred to as “fruit sugar,” honey, unrefined sweeteners, syrups, such as agave syrup or agave nectar, maple syrup, corn syrup and high fructose corn syrup (or HFCS).

As used herein, the term “sweetener enhancer” refers to a compound (or composition) capable of enhancing or intensifying sensitivity of the sweet taste. The term “sweetener enhancer” is synonymous with a “sweetness enhancer,” “sweet taste potentiator,” “sweetness potentiator,” and/or “sweetness intensifier.” A sweetener enhancer enhances the sweet taste, flavor, mouth feel and/or the taste profile of a sweetener without giving a detectable sweet taste by the sweetener enhancer itself at an acceptable use concentration. In some embodiments, the sweetener enhancer provided herein may provide a sweet taste at a higher concentration by itself. Certain sweetener enhancers provided herein may also be used as sweetening agents.

Sweetener enhancers can be used as food additives or flavors to reduce the amounts of sweeteners in foods while maintaining the same level of sweetness. Sweetener enhancers work by interacting with sweet receptors on the tongue, helping the receptor to stay switched “on” once activated by the sweetener, so that the receptors respond to a lower concentration of sweetener. These ingredients could be used to reduce the calorie content of foods and beverages, as well as save money by using less sugar and/or less other sweeteners. Examples of sweetener enhancers include, but are not limited to, brazzein, miraculin, curculin, pentadin, mabinlin, thaumatin, and mixtures thereof.

In some cases, sweeteners can be used as sweetener enhancers or flavors when their dosages in food and beverage are low. In some cases, sweetener enhancers can be utilized as sweeteners where their dosages in foods and beverages are higher than dosages regulated by FEMA, EFSA or other related authorities.

As used herein, the phrase “low sweetness products produced by synthesis, fermentation or enzymatic conversion” refers to products that have less sweetness or similar sweetness than sucrose. Examples of low sweetness products produced by extraction, synthesis, fermentation or enzymatic conversion method include, but are not limited to, sorbitol, xylitol, mannitol, erythritol, trehalose, raffinose, cellobiose, tagatose, DOLCIA PRIMA™ allulose, inulin, N--[N-[3-(3-hydroxy-4-methoxyphenyl)propyl]-alpha-aspartyl]-L-phenylalanine 1-methyl ester, glycyrrhizin, and mixtures thereof.

For example, “sugar alcohols” or “polyols” are sweetening and bulking ingredients used in manufacturing of foods and beverages. As sugar substitutes, they supply fewer calories (about a half to one-third fewer calories) than sugar, are converted to glucose slowly, and are not characterized as causing spiked increases in blood glucose levels.

Sorbitol, xylitol, and lactitol are exemplary sugar alcohols (or polyols). These are generally less sweet than sucrose, but have similar bulk properties and can be used in a wide range of food and beverage products. In some case, their sweetness profile can be fine-tuned by being mixed together with high-intensity sweeteners.

The terms “flavor” and “flavor characteristic” are used interchangeably with reference to the combined sensory perception of one or more components of taste, aroma, and/or texture.

The terms “flavoring agent”, “flavoring” and “flavorant” are used interchangeably with reference to a product added to food or beverage products to impart, modify, regulate or enhance the flavor of food. As used herein, these terms do not include substances having an exclusively sweet, sour, or salty taste (e.g., sugar, vinegar, and table salt).

The term “natural flavoring substance” refers to a flavoring substance obtained by physical processes that may result in unavoidable but unintentional changes in the chemical structure of the components of the flavoring (e.g., distillation and solvent extraction), or by enzymatic or microbiological processes, from material of plant or animal origin.

The term “synthetic flavoring substance” refers to a flavoring substance formed by chemical synthesis.

The term “regulate,” as used herein, includes reducing, enhancing or modifying the sensory perception of a flavor characteristic without changing the nature or quality thereof.

The term “enhance,” as used herein, includes augmenting, intensifying, accentuating, magnifying, and potentiating the sensory perception of a flavor characteristics.

Unless otherwise specified, the terms “modify” or “modified” as used herein, includes altering, varying, suppressing, depressing, fortifying and supplementing the sensory perception of a flavor characteristic where the quality or duration of such characteristic was deficient.

The phrase “sensory profile” or “taste profile” is defined as the temporal profile of all basic tastes of a sweetener. The onset and decay of sweetness when a sweetener is consumed, as perceived by trained human tasters and measured in seconds from first contact with a taster’s tongue (“onset”) to a cutoff point (typically 180 seconds after onset), is called the “temporal profile of sweetness.” A plurality of such human tasters is called a “sensory panel”. In addition to sweetness, sensory panels can also judge the temporal profile of the other “basic tastes”: bitterness, saltiness, sourness, piquance (aka spiciness), and umami (aka savoriness or meatiness). The onset and decay of bitterness when a sweetener is consumed, as perceived by trained human tasters and measured in seconds from first perceived taste to the last perceived aftertaste at the cutoff point, is called the “temporal profile of bitterness”.

The phrase “sucrose equivalence” or “SugarE” is the amount of non-sucrose sweetener required to provide the sweetness of a given percentage of sucrose in the same food, beverage, or solution. For instance, a non-diet soft drink typically contains 12 grams of sucrose per 100 ml of water, i.e., 12% sucrose. This means that to be commercially accepted, diet soft drinks must generally have the same sweetness as a 12% sucrose soft drink, i.e., a diet soft drink must have a 12% SugarE. Soft drink dispensing equipment assume an SugarE of 12%, since such equipment is set up for use with sucrose-based syrups.

As used herein, the term “off-taste” refers to an amount or degree of taste that is not characteristically or usually found in a beverage product or a consumable product of the present disclosure. For example, an off-taste is an undesirable taste of a sweetened consumable to consumers, such as, a bitter taste, a licorice-like taste, a metallic taste, an aversive taste, an astringent taste, a delayed sweetness onset, a lingering sweet aftertaste, and the like, etc.

The term “orally consumable product” refers to a composition that can be drunk, eaten, swallowed, inhaled, ingested or otherwise in contact with the mouth or nose of man or animal, including compositions which are taken into and subsequently ejected from the mouth or nose. Orally consumable products are safe for human or animal consumption when used in a generally acceptable range.

As used herein, the term “fruit” refers to firm fruits, soft fruits, sliced pieces with skin remaining, and/or dried/scarified/pricked/scraped fruit, which are well-known in the art, and described herein. Examples of fruit include, but are not limited to, apple, pear, orange, tangerine, lemon, lime, apricot, plum, prune, kiwi, guava, pineapple, coconut, papaya, mango, grape, cherry, pomegranate, grape fruit passion fruit, dragon fruit, melons and berries. Example of berries include, but are not limited to, cranberry, blueberry, boysenberry, elderberry, chokeberry, lingonberry, raspberry, mulberry, gooseberry, huckleberry, strawberry, blackberry, cloudberry, blackcurrant, redcurrant and white currant. Exemplary melons include, but are not limited to, watermelon, cantaloupe, Muskmelon, honeydew melon, canary melon, casaba melon, chareatais melon, crenshaw melon, galia melon, golden Langkawi melon, hami melon, honey globe melon, horned melon, jadedew melon, kantola melon and Korean melon.

The term “fruit juice” refers to a juice derived from one or more fruits. Fruit juices include freshly prepare fruit juices, concentrated fruit juices, and juices reconstituted from concentrated fruit juices.

The term “vegetables” refers to fresh vegetables, preserved vegetables, dried vegetables, vegetable juice and vegetable extracts. Examples of vegetables include, but are not limited to, broccoli, cauliflower, artichokes, capers, cabbage, turnip, radish, carrot, celery, parsnip, beetroot, lettuce, beans, peas, potato, eggplant, tomato, sweet corn, cucumber, squash, zucchinis, pumpkins, onion, garlic, leek, pepper, spinach, yam, sweet potato, taro, and yams and cassava.

The term “vegetable juice” refers to a juice derived from one or more vegetables. Vegetables juices include freshly prepare vegetables juices, concentrated vegetables juices, and juices reconstituted from concentrated vegetables juices.

As used herein, the term “salt” refers to a chemical compound consisting of an ionic assembly of positively charged cations and negatively charged anions, which results in a compound with no net electric charge. A common example is table salt, with positively charged sodium ions and negatively charged chloride ions. The salt in this specification means all edible salts, including but not limited to NaCl based salts such as table salt, kosher salt, Himalayan salt, sea salt, Hawaiian salt, flake salt, pickling salt and iodionized salt, MgCh, CaCl₂, KCl, and trisodium citrate.

Unless otherwise noted, the term “ppm” (parts per million) means parts per million on a v/v or wt/wt basis.

### II. Compositions of the Present Application

Traditionally, chemical compounds such as menthol, menthone, methyl acetate and essential oils such as peppermint oil have been used to provide a cooling effect both orally and topically. These products are used in applications such as body wash, shampoo, mouthwash, toothpaste, chewing gum, mint confection, cooling ointment, medicated oils, insect repellent etc. However, these products have certain drawbacks. When conventional cooling agents, such as menthol, are present in compositions in excess, a bitter or burn feeling may be produced, making the final products undesirable. There is need to develop natural cooling agents or regulators to meet market taste requirements. The inventor has surprisingly found that combining composition in this invention could regulate the intensity and longevity of coolingness and provide a palatable taste.

Transient receptor potential melastatin 8 (TRPM8) is a polymodal, Ca²⁺permeant, and non-selective cation channel, identified as the physiological sensor of environmental cold. TRPM8 channels are mainly expressed in a subpopulation of sensitive primary afferent neurons, which innervate highly cold-sensitive tissues, including skin, the oral cavity, epithelium, teeth, nasal mucosa, tongue, and cornea. The oral cavity includes oral cavity includes the lips, hard palate (the bony front portion of the roof of the mouth), soft palate (the muscular back portion of the roof of the mouth), retromolar trigone (the area behind the wisdom teeth), front two-thirds of the tongue, gingiva (gums), buccal mucosa (the inner lining of the lips and cheeks), and floor of the mouth under the tongue. TRPM8 channels are also expressed in the visceral tissues innervated by pelvic or vagal nerves in e.g., the bladder, male urinary, genital tract, spermatic chord, colon, and pulmonary tissues.

TRPM8 channels can be regulated by natural and synthetic cooling mimetic agents, such as icilin, eucalyptol, menthol and its derivatives, camphor, eucalyptol, linalool, geraniol, hydroxycitronellal, cooling agent 10, CPS-113, CPS-369, Frescolat ML, Frescolat MGA, and an abundance of agonists, such as WS-12. The inventor has surprisingly found that compositions of the present application regulate a cooling effect or taste. While not wishing to be bound by theory, it is believed that the components in the compositions of the present application, including both the one or more NHIS and/or derivatives thereof, and conventional cooling agents could regulate the TRPM8 channel.

In one aspect, the present application provides a composition comprising one or more NHIS and/or derivatives thereof that regulate a coolness taste, feeling or flavor of the composition, wherein the one or more high intensity natural sweetener and/or derivative thereof are present in the composition in a total amount sufficient for regulating a cooling effect or taste. In some embodiments, the composition of the present application provides or enhances a cooling effect or taste.

In some embodiments, the composition of the present application comprises one or more NHIS and/or derivatives thereof selected from the group consisting of stevia extracts (SEs), glycosylated stevia extracts (G-SEs), stevia extract-MRPs (SE-MRPs), glycosylated stevia extract-MRPs (G-SE-MRPs), stevia glycosides (SGs), glycosylated stevia glycosides (GSGs), stevia glycoside-MRPs (SG-MRPs), glycosylated stevia glycoside-MRPs (GSG-MRPs), sweet tea extracts (STEs), glycosylated sweet tea extracts (G-STEs), sweet tea extract-MRPs (STE-MRPs), glycosylated sweet tea extract-MRPs (G-STE-MRPs), sweet tea glycosides (STGs), glycosylated sweet tea glycosides (G-STGs), sweet tea glycoside-MRPs (STG-MRPs), glycosylated sweet tea glycoside-MRPs (G-STG-MRPs), monk fruit extracts (MFEs), glycosylated monk fruit extracts (G-MFEs), monk fruit glycosides (MFGs), glycosylated monk fruit glycosides (G-MFGs), monk fruit glycoside-MRPs (MFG-MRPs), and glycosylated monk fruit glycoside-MRPs (G-MFG-MRPs).

In certain preferred embodiments, the one or more NHIS and/or derivatives thereof is further combined with one or more cooling agent(s).

Without wishing to be bound by theory, it is believed that the one or more NHIS and/or derivatives thereof of the present application can increase the solubility of cooling agents, improve their penetrating capability and prolong their retention time in the mucous membranes present in the oral cavities, including e.g., mouth, nose, breathing tube, lung, and skin, thereby lowering their threshold for achieving a cool feeling.

Taste receptors are normally regarded by location on the tongue, where they are expressed in taste cells of taste buds. However, taste receptors have been identified in other tissues ranging from the lungs and guts to the brain. These so called “extra-oral” taste receptors play hidden roles such as local chemoreceptors in the body and local effects on innate immunity. For instance, bitter taste receptors TAS2Rs are expressed not only in the oral cavity but also in many extraoral tissues including gastrointestine, nasal epithelium, airway, skin, heart and brain.

The compositions of the present application can regulate both taste and flavor sensory receptors located in mouth cavity, nose cavity, breathing airway and food tubes. On the tongue, bitter (T2R), sweet (T1R2/T1R3), and Umami (T1R1/T1R3) receptors are expressed in distinct cells of the taste bud called type II cells. Each taste cell can detect one type of taste (unisensory), and coupling of that cell to different afferent gustatory neurons dictates how the response is perceived by the brain. There are also multi-taste sensory receptors in a single cell such as bitter and sweet taste receptors co-expressed together in many extraoral chemosensory cell types, such as intestinal tuft cells and solitary chemosensory cells.

In certain embodiments, glycoside components of the present application may regulate one or more multi-taste sensory receptors, such as TRPM5, TRPM8, T1R2, T1R3, T1R1/T1R3, TAS2R4 etc. This can potentiate the activities of sweet, umami, bitter, salt, cooling channel simultaneously. Contrary to current prevailing knowledge that single stimuli regulates single taste sensation and transfers the resulting signals parallel with other taste sensation to brain for final integration of overall flavor and taste via neuronal perceptive pathways, it is believed that stimuli resulting from the glycoside components of the present application can regulate multi-taste sensations and transfer multi-signals therefrom parallel to brain, and can further regulate di, tri, tetra etc. multi-functional taste receptors, such as e.g., T1R1/TIR3 receptors responsible for sweet and umami, and sweet/bitter taste receptors in solitary chemosensory cells and transfer the signals therefrom via local congruent neurons. For instance, SG-MRPs can regulate sweet, bitter, umami, kokumi taste receptors and also the di, tri, tetra receptors, as well as flavor receptors in the olfactory bulb. It is essential to have products that can regulate at least two distinct taste receptors and their co-taste receptors (such as T1R1/T1R3) in formulation to make food and beverages more palatable. Compositions of the present application can produce different distinct stimuli and harmonize the overall flavor and taste of foods and beverages.

In one embodiment, the composition comprises one or more NHIS and/or derivatives thereof in a total amount sufficient for regulating a taste receptor selected from the group consisting of TRPM5, T1R2, T1R3, T1R1/T1R3, and TAS2R4.

The inventor of the present application has discovered that a synergistic cooling effect may be achieved when combining the one or NHIS and/or derivatives thereof of the present application with one or more cooling agents. These combined products can regulate the cooling profile of compositions containing only one or the other, including the intensity and duration of coolness, and cooling area of cooling agents, such as menthol.

Accordingly, in another embodiment, the composition includes the one or more NHIS and/or derivatives thereof in combination with one or more natural or synthetic cooling agent(s).

In certain embodiments, the one or more one or more NHIS and/or derivatives thereof and one or more natural or synthetic cooling agents are collectively present in amount(s) sufficient for synergistically regulating a cooling effect or taste. In some embodiments, the one or more one or more NHIS and/or derivatives thereof and one or more natural or synthetic cooling agents are collectively present in amount(s) sufficient for synergistically providing or enhancing a cooling effect or taste and the degree of enhancement is at least 10%, at least 20%, at least 30%, at least 50%, at least 75%, at least 100%, at least 2-fold, at least 5-fold or at least 10-fold greater than a corresponding level obtained by an otherwise identical composition containing the one or more NHIS and/or derivatives thereof without the cooling agent, or an otherwise identical composition containing the cooling agent without the one or more NHIS and/or derivatives thereof.

In some embodiments, the natural or synthetic cooling agent is present in the composition in an amount sufficient to mask a bitter flavor or an off-note in one or more NHIS and/or derivatives thereof.

In some embodiments, the one or more NHIS and/or derivatives thereof is present in the composition in a total amount sufficient to regulate a cooling effect. In some embodiments, the one or more NHIS and/or derivatives thereof is present in the composition in a total amount sufficient to provide or enhance a cooling effect by an otherwise identical composition comprising the natural or synthetic cooling agent without the one or more NHIS and/or derivatives thereof.

In some embodiments, the cooling agent is selected from icilin, eucalyptol, camphor, eucalyptol, linalool, geraniol, hydroxycitronellal, menthoxypropanediol (cooling agent 10), CPS-113, CPS-369, Frescolat ML, Frescolat MGA, and combinations thereof.

In some embodiments, the cooling agent isselected from menthol, menthone, menthol esters, ethers, menthane carboxamides, non-menthol-derived coolants, derivative therefrom and combinations thereof.

In some embodiments, the cooling agent is an ether having a FEMA number selected from the group consisting of 3748, 3805, 3806, 3807, 3808, 3810, 4006, 4327, 4308, 4155, 3784, 3849, 4053, 4054, 4154, 4230, 4285, 4497, and 4604.

In some embodiments, the cooling agent is a menthane carboxamide selected from the group consisting of WS-3, WS-5, WS-10, WS-11, WS-12, WS-14, WS-33, WS-34, WS-35, WS-56, WS-61, WS-63, WS-108, WS-109, WS-134, WS-138, and combinations thereof.

In some embodiments, the cooling agent is a non-menthol derived coolant with a FEMA number selected from the group consisting of 2962, 3804, 4557, 4603, and 4602.

In some embodiments, the weight ratio (w/w) of the one or more NHIS and/or derivatives thereof to the one or more cooling agent(s) ranges from about 99:1 to 1:2, 99:1 to 1:1, 99:1 to 2:1, 99:1 to 5:1, 99:1 to 10:1, 99:1 to 20:1, 99:1 to 40:1, 99:1 to 60:1, 99:1 to 80:1, 80:1 to 1:2, 80:1 to 1:1, 80:1 to 2:1, 80:1 to 5:1, 80:1 to 10:1, 80:1 to 20:1, 80:1 to 40:1, 80:1 to 60:1, 60:1 to 1:2, 60:1 to 1:1, 60:1 to 2:1, 60:1 to 5:1, 60:1 to 10:1, 60:1 to 20:1, 60:1 to 40:1, 40:1 to 1:2, 40:1 to 1:1, 40:1 to 2:1, 40:1 to 5:1, 40:1 to 10:1, 40:1 to 20:1, 20:1 to 1:2, 20:1 to 1:1, 20:1 to 2:1, 20:1 to 5:1, 20:1 to 10:1, 10:1 to 1:2, 20:1 to 1:1, 20:1 to 2:1, 20:1 to 5:1, 20:1 to 10:1, 10:1 to 1:1, 10:1 to 2:1, 10:1 to 5:1, 5:1 to 1:2, 5:1 to 1:1, 5:1 to 2:1, 2:1 to 1:2, 2:1 to 1:1, or 1:1 to 1:2.

In some embodiments, the weight ratio (w/w) of the one or more NHIS and/or derivatives thereof to the one or more cooling agent(s) ranges from about 100:1, 80: 1, 60: 1, 40:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:80, 1:100, or any ratio derived from any two of the aforementioned integers.

In some embodiments, the composition further comprises at least one additional steviol glycoside or steviol glycoside extract described in the present application.

In some embodiments, the composition further comprises at least one flavoring substance. In some embodiments, the flavoring substance is a fruit juice or a vegetable juice.

In another aspect, the present application provides a consumable product containing any of the compositions described herein.

In some embodiments, the consumable product containing the composition is a food product or foodstuff as e.g., described in the present application. In certain particular embodiments, the food product (or foodstuff) is a confection, a condiment, a baked good, a cereal composition, a dairy product, a chewing composition, a table-top sweetener or any other specific food product or foodstuff as described in the present application.

In other embodiments, the consumable product containing the composition is a beverage product as e.g., described in the present application. In certain particular embodiments, the beverage product is selected from the group consisting of carbonated beverages (e.g., frozen carbonated beverages, enhanced sparkling beverages, cola, fruit-flavored sparkling beverages (e.g., lemon-lime, orange, grape, strawberry and pineapple), ginger-ale, soft drinks and root beer); non-carbonated beverages (e.g., fruit juice, fruit-flavored juice, juice drinks, nectars, vegetable juice, vegetable-flavored juice, sports drinks, energy drinks, enhanced water drinks, enhanced water with vitamins, near water drinks (e.g., water with natural or synthetic flavorants), coconut water, tea type drinks (e.g., black tea, green tea, red tea, oolong tea), coffee, cocoa drink, broths, beverages comprising milk components (e.g., milk beverages, coffee comprising milk components, cafe au lait, milk tea, fruit milk beverages), beverages comprising cereal extracts, and smoothies; frozen beverages, semi-frozen (“slush”) beverages, non-frozen beverages, concentrated beverages (e.g., powdered, frozen, or syrup), dairy beverages, non-dairy beverages, probiotic beverages, prebiotic beverages, herbal beverages, non-herbal beverages, caffeinated beverages, non-caffeinated beverages, alcoholic beverages, non-alcoholic beverages, flavored beverages, non-flavored beverages, vegetable-based beverages, fruit-based beverages, root/tuber/corm-based beverages, nut-based beverages, other plant-based beverages, cola-based beverages, chocolate-based beverages, meat-based beverages, seafood-based beverages, other animal-based beverages, algae-based beverages, calorie enhanced beverages, calorie-reduced beverages, calorie-free beverages, and combinations thereof, including any specific food product or foodstuff described in the present application.

In other embodiments, the consumable product containing the composition is e.g., a pharmaceutical product, an oral hygiene product, a cosmetic product, smokable product or another consumable product as described in the present application.

Exemplary oral hygiene products include, but are not limited to those selected from the group consisting of mouthwashes, mouth rinses, breath fresheners, toothpastes, tooth polishes, dentifrices, mouth sprays, teeth whitening agents, soaps, and perfumes.

Exemplary cosmetic products include, but are not limited to those selected from the group consisting of cosmetic wipes, soaps, synthetic detergents, liquid wash compositions, shower preparations, bath preparations, bath products (e.g., capsule, oil, tablet, salt, bath salt, soap, etc.), effervescent preparations, skin care products (e.g., emulsions (as described above), ointments, pastes, gels (as described above), oils, balsams, serum, powders (e.g., face powder, body powder), masks, pencils, sticks, roll-ons, pumps, aerosols (foaming, non-foaming or post-foaming), deodorants, antiperspirants, mouthwashes, mouth rinses, foot care products (including keratolytic, deodorant), insect repellents, sunscreens, aftersun preparations, shaving products, aftershave balms, pre- and aftershave lotions, depilatory agents, hair care products (e.g., shampoos (including 2-in-1 shampoos, anti-dandruff shampoos, baby shampoos, shampoos for dry scalps, concentrated shampoos), conditioners, hair tonics, hair water?, hair rinses, styling cremes, pomades, perm and setting lotions, hair sprays, styling aids (e.g., gels or waxes), hair smoothing agents (e.g., detangling agents, relaxers), hair dyes (e.g., temporary direct-dyeing hair dyes, semi-permanent hair dyes, permanent hair dyes, hair conditioners, hair mousses, eye care products, make-up, make-up removers and baby products.

Exemplary smokable products include, but are not limited to those selected from the group consisting of tobacco products (e.g., cigarettes, electronic cigarettes (e-cigarettes), cigars, pipe and cigar tobaccos, chew tobacco, vaporizable liquids, particular forms of tobacco (e.g., shredded filler, leaf, stem, stalk, homogenized leaf cured, reconstituted binders, reconstituted tobacco from tobacco dust, fines, or other sources in sheet, pellet or other forms); cannabis products (e.g., flower materials, leaf materials, extracts, oils, edible candies, vaporizable liquids, cannabis-infused beverages, etc.); smokable materials burned to provide desirable aromas (e.g., charcoal briquettes for grilling foods, incense etc.); (e.g., flower materials, leaf materials, extracts, oils, edible candies, vaporizable liquids, cannabis-infused beverages, etc.); tobacco substitutes formulated from non-tobacco materials; and combinations thereof.

In one embodiment, an alcohol-containing consumable product includes a composition in accordance with the present application to relieve the burn feeling of alcohol in the mouth or throat.

In some embodiments, a consumable product includes a composition of the present application to relieve pain or discomfort resulting from chili or other warm, stinging substances.

In some embodiments, the composition of the present application comprises the one or more NHIS and/or derivatives thereof, and/or the one or more cooling agents, individually or collectively, in an amount ranging from 1 ppm to 15,000 ppm, from 1 ppm to 10,000 ppm, from 1 ppm to 5,000 ppm, from 10 ppm to 1,000 ppm, from 50 ppm to 900 ppm, from 50 ppm to 600 ppm, from 50 ppm to 500 ppm, from 50 ppm to 400 ppm, from 50 ppm to 300 ppm, from 50 ppm to 200 ppm, from 100 ppm to 600 ppm, from 100 ppm to 500 ppm, from 100 ppm to 400 ppm, from 100 ppm to 300 ppm, from 100 ppm to 200 ppm, from 125 ppm to 600 ppm, from 125 ppm to 500 ppm, from 125 ppm to 400 ppm, from 125 ppm to 300 ppm, from 125 ppm to 200 ppm, from 150 ppm to 600 ppm, from 150 ppm to 500 ppm, from 150 ppm to 500 ppm, from 150 ppm to 400 ppm, from 150 ppm to 300 ppm, from 150 ppm to 200 ppm, from 200 ppm to 600 ppm, from 200 ppm to 500 ppm, from 200 ppm to 400 ppm, from 200 ppm to 300 ppm, from 300 ppm to 600 ppm, from 300 ppm to 500 ppm, from 300 ppm to 400 ppm, from 400 ppm to 600 ppm, from 500 ppm to 600 ppm, from 20 ppm to 200 ppm, from 20 ppm to 180 ppm, from 20 ppm to 160 ppm, from 20 ppm to 140 ppm, from 20 ppm to 120 ppm, from 20 ppm to 100 ppm, from 20 ppm to 80 ppm, from 20 ppm to 60 ppm, from 20 ppm to 40 ppm, from 40 ppm to 150 ppm, from 40 ppm to 130 ppm, from 40 ppm to 100 ppm, from 40 ppm to 90 ppm, from 40 ppm to 70 ppm, from 40 ppm to 50 ppm, from 20 ppm to 100 ppm, from 40 ppm to 100 ppm, from 50 ppm to 100 ppm, from 60 ppm to 100 ppm, from 80 ppm to 100 ppm, from 5 ppm to 100 ppm, from 5 ppm to 95 ppm, from 5 ppm to 90 ppm, from 5 ppm to 85 ppm, from 5 ppm to 80 ppm, from 5 ppm to 75 ppm, from 5 ppm to 70 ppm, from 5 ppm to 65 ppm, from 5 ppm to 60 ppm, from 5 ppm to 55 ppm, from 5 ppm to 50 ppm, from 5 ppm to 45 ppm, from 5 ppm to 40 ppm, from 5 ppm to 35 ppm, from 5 ppm to 30 ppm, from 5 ppm to 25 ppm, from 5 ppm to 20 ppm, from 5 ppm to 15 ppm, from 5 ppm to 10 ppm.

In some embodiments, the composition of the present application comprises the one or more NHIS and/or derivatives thereof, and/or the one or more cooling agents, individually or collectively, in an amount of 1-99 wt%, 1-95 wt%, 1-90 wt%, 1-80 wt%, 1-70 wt%, 1-60 wt%, 1-50 wt%, 1-40 wt%, 1-30 wt%, 1-20 wt%, 1-10 wt%, 1-5 wt%, 5-99 wt%, 5-95 wt%, 5-90 wt%, 5-80 wt%, 5-70 wt%, 5-60 wt%, 5-50 wt%, 5-40 wt%, 5-30 wt%, 5-20 wt%, 5-10 wt%, 10-99 wt%, 10-95 wt%, 10-90 wt%, 10-80 wt%, 10-70 wt%, 10-60 wt%, 10-50 wt%, 10-40 wt%, 10-30 wt%, 10-20 wt%, 20-99 wt%, 20-95 wt%, 20-90 wt%, 20-80 wt%, 20-70 wt%, 20-60 wt%, 20-50 wt%, 20-40 wt%, 20-30 wt%, 30-99 wt%, 30-95 wt%, 30-90 wt%, 30-80 wt%, 30-70 wt%, 30-60 wt%, 30-50 wt%, 30-40 wt%, 40-99 wt%, 40-95 wt%, 40-90 wt%, 40-80 wt%, 40-70 wt%, 40-60 wt%, 40-50 wt%, 50-99 wt%, 50-95 wt%, 50-90 wt%, 50-80 wt%, 50-70 wt%, 50-60 wt%, 60-99 wt%, 60-95 wt%, 60-90 wt%, 60-80 wt%, 60-70 wt%, 70-99 wt%, 70-95 wt%, 70-90 wt%, 70-80 wt%, 80-99 wt%, 80-95 wt%, 80-90 wt%, 90-99 wt%, 90-95 wt%, 95-99 wt% of the composition.

In some embodiments, the composition of the present application comprises the one or more NHIS and/or derivatives thereof, and/or the one or more cooling agents, individually or collectively, in an amount of at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, at least 99 wt % of the composition.

In some embodiments, the one or more NHIS and/or derivatives thereof comprise one or more SGs and/or one or more non-SG components selected from the group consisting of sweet tea extracts, monk fruit extracts, suaviosides and mogrosides. In some embodiments, the one or more NHIS and/or derivatives thereof comprise thaumatin.

In some embodiments, the composition of the present application further comprises one or more SHIS selected from the group consisting of cyclamates and salts thereof, aspartame, saccharin and salts thereof, glycyrrhizin, sucralose, xylitol, acesulfame-K, neotame, N--[N-[3-(3-hydroxy-4-methoxyphenyl) propyl]-alpha-aspartyI]-L-phenylalanine 1-methyl ester (ANS9801), monellin, brazzein, miraculin, curculin, pentadin and mabinlin.

In some embodiments, the composition of the present application further comprises one or more flavonoid glycosides, isoflavone glycosides, saponin glycosides, phenol glycosides, cynophore glycosides, anthraquinone glycosides, cardiac glycosides, bitter glycosides, coumarin glycosides, or sulfur glycosides.

Exemplary flavonoids include, but are not limited to, anthocyanidins; anthoxanthins, including flavones, such as luteolin, apigenin, tangeritin; and flavonols, such as quercetin, kaempferol, myricetin, fisetin, galangin, isorhamnetin, pachypodol, rhamnazin, pyranoflavonols, furanoflavonols; flavanones, such as hesperetin, naringenin, eriodictyol, and homoeriodictyol; flavanonols, such as taxifolin (or dihydroquercetin) and dihydrokaempferol; and flavans, including flavanols, such as catechin, gallocatechin, catechin 3-gallate, gallocatechin 3-gallate, epicatechin, epigallocatechin (EGC), epicatechin 3-gallate, epigallocatechin 3-gallate, theaflavin, theaflavin-3′-gallate, theaflavin-3,3′-digallate, thearubigin, and proanthocyanidins, which are dimers, trimers, oligomers, or polymers of the flavanols, and glycosides thereof.

Exemplary isoflavonoids include isoflavones, such as genistein, daidzein, glycitein; isoflavanes, isoflavandiols, isoflavenes, coumestans, pterocarpans, and glycosides thereof.

In some embodiments, the composition of the present application further comprises one or more polyphenols. Exemplary polyphenols include gallic acid, ellagic acid, quercetin, isoquercitrin, rutin, citrus flavonoids, catechins, proanthocyanidins, procyanidins, anthocyanins, reservatrol, isoflavones, curcumin, hesperidin, naringin, and chlorogenic acid, and glycosides thereof.

In some embodiments, the composition further comprises one or more tannins. Exemplary tannins include gallic acid esters, ellagic acid esters, ellagitannins, including rubusuaviins A, B, C, D, -E, and -F; punicalagins, such as pedunculagin and 1(β)-O-galloyl pedunculagin; strictinin, sanguiin H-5, sanguiin H-6, 1-desgalloyl sanguiin H-6. lambertianin A, castalagins, vescalagins, castalins, casuarictins, grandimins, punicalins, roburin A, tellimagrandin II, terflavin B; gallotannins, including digalloyl glucose and 1,3,6-trigalloyl glucose; flavan-3-ols, oligostilbenoids, proanthocyanidins, polyflavonoid tannins, catechol-type tannins, pyrocatecollic type tannins, flavolans, and glycosides thereof.

In some embodiments, the composition of the present application further comprises one or more carotenoids. Exemplary carotenoids include carotenes, including α-, β-, γ-, δ-, and ε-carotenes, lycopene, neurosporene, phytofluene, phytoene; and xanthophylls, including canthaxanthin, cryptoxanthin, zeaxanthin, astaxanthin, lutein, rubixanthin, and glycosides thereof.

In some embodiments, the composition of the present application further comprises one or more diterpenes, diterpenoids, triterpenes and/or triterpenoids. Exemplary diterpenes and diterpenoids include steviol, ent-16α,17-dihydroxy-kaurane-19-oic acid, ent-13-hydroxy-kaurane-16-en-19-oic acid, ent-16β,17-dihydroxy-kaurane-3-one, ent-16α,17-dihydroxy-kaurane-19-oic acid, ent-16α,17-dihydroxy-kaurane-3-one, ent-kaurane-3α,16β,17-3-triol, ent-13,17-dihydroxy-kaurane-15-en-19-oic acid, and glycosides thereof. Exemplary triterpenes and triterpenoids, include oleanolic acid, ursolic acid, saponin, and glycoside thereof.

In some embodiments, the composition of the present application comprises at least one flavoring substance, such as a fruit juice or vegetable juice.

In some embodiments, the composition of the present application further comprises one or more warming, bitter or tingling substances.

Another aspect of the present application relates to a method of improving the taste profile of a consumable (such as a food product or beverage). The method comprises the step of adding to the consumable, a sufficient amount of the composition of the present application. In some embodiments, the method further includes adding to the consumable at least one flavoring substance in an amount sufficient to improve a flavor profile of the food product or beverage.

Another aspect of the present application relates to a method for improving a cooling profile of a composition. The method comprises the step of adding one or more NHIS and/or derivatives thereof to the composition in an amount sufficient to enhance a cooling effect or taste. In some embodiments, the composition comprises one or more warming, bitter or tingling agents.

In another aspect, a method for improving a cooling profile of a composition. The method comprises the step of adding one or more NHIS and/or derivatives thereof and one or more cooling agent to the composition in an amount sufficient to enhance a cooling effect or taste. In certain particular embodiments, the composition comprises one or more warming, bitter or tingling agents.

In some embodiments, the compositions of the present application may be used for medical treatment in the pharmaceutical arts for e.g., increasing production of tears for relief of dry eye discomfort; reducing blood glucose levels; reducing weight gain, or fat depot levels; and providing treatments for metabolic syndrome, obesity, prediabetes, and type II diabetes, cancer, bladder weakness, hypercholesterolemia, hypertension, coronary heart disease, diabetic neuropathy and retinopathy, erectile dysfunction, kidney disease, and pancreatitis, among others.

In summary, there are at least other two types of neuronal perceptive pathways: groups of substances, including those described in the compositions of the present application, a single stimuli could regulate multi-independent single-taste receptors (sensing one taste only such as sweet or bitter, or umami or cooling) simultaneously, it could regulate the signals of cell to cell communication, attenuates signals by inputting different concentration of stimuli. There are groups of substances, a single stimuli could mainly regulate the multi-taste receptors (both sweet and bitter, both sweet and umami, combination of sweet/bitter/umami etc.) and transfer the signals via congruent neurons to the brain. Like multi-sensory interactions in connection with the ventriloquism effect, in which the spatial properties of stimuli presented in one modality can influence the spatial content of perception in another modality simultaneously, the inventor believes that different basic tastes, including sweet, bitter and umami also interact with each other in the course of neuronal perceptive pathways through congruent neurons before final integration of overall taste and flavor is achieved. There are spatially distributed neurons in the neuronal perceptive pathways, where both neurons respond to single-taste signals or neurons response to multi-taste signals present.

### A. Nature High Intensity Sweetener (NHIS) and Glycosylated NHIS

In some embodiments, the composition of the present application comprises one or more NHIS and/or glycosylated NHIS.

NHISs of the present application include, but are not limited to, Stevia extracts (SEs), steviol glycosides (SGs), sweet tea extracts (STE), sweet tea glycosides (STGs) such as sauviosides, monk fruit extracts (MFEs) and morgrosides.

Some of the SGs for use in the compositions and consumable products described herein include those described in Table B-2. In Table B-2, the phrase “# Added sugar moieties” means sugar moieties added to the steviol or isosteviol backbone. The “added sugar moieties” are native to the respective steviol glycoside and are NOT sugar groups added in an exogenous glycosylation reaction.

TABLE B-2

Exemplary steviol glycosides (SGs)

| SG Name | MW | # Added Glucose moieties mw=180 | # Added Rhamnose /Deoxy-hexose moieties mw=164 | # Added Xylose/ Arabinose moieties mw=150 | R1 (C-19) | R2 (C-13) | Backbone |
|---|---|---|---|---|---|---|---|
| Related SvGn# 1 | 457 | - | | | | | |
| Steviolmonoside | 479 | 1 | | | H- | Glcβ1- | Steviol |
| Steviolmonoside A | 479 | 1 | 1 | | Glcβ1- | H- | |
| SG-4 | 611 | 1 | | 1 | H- | Xylβ(1-2)Glcβ1- | Steviol |
| Dulcosid e Al | 625 | 1 | 1 | | H- | Rhaα(1-2)Glcβ1- | Steviol |
| Isosteviolbioside | 641 | 2 | | | H- | Glcβ(1-2)Glcβ1- | Isosteviol |
| Reb-G 1 | 641 | 2 | | | H- | Glcβ(1-3)Glcβ1- | Steviol |
| Rubusosi de | 641 | 2 | | | Glcβ1- | Glcβ1- | Steviol |
| Steviolbi oside | 641 | 2 | | | H- | Glcβ(1-2)Glcβ1- | Steviol |
| Related SvGn#3 | 675 | – | | | | | |
| Reb-Fl | 773 | 2 | | 1 | H- | Xylβ(1-2)[Glcβ(1-3)]Glcβ1- | Steviol |
| Reb-Rl | 773 | 2 | | 1 | H- | Glcβ(1-2)[Glcβ(1-3)]Xylβ1- | Steviol |
| Steviosid e F (SG-1) | 773 | 2 | | 1 | Glcβ1- | Xylo(1-2)Glcβ1- | Steviol |
| SG-Unkl | 773 | 2 | | 1 | - | - | Steviol |
| Dulcosid e A | 787 | 2 | 1 | | Glcβ1- | Rhaα(1-2)Glcβ1- | Steviol |
| Dulcosid eB (JECFA C) | 787 | 2 | 1 | | H- | Rhaα(1-2)[Glcβ(1-3)]Glcβ1- | Steviol |
| SG-3 | 787 | 2 | 1 | | H- | 6-deoxyGlcβ(1-2)[Glcβ(1-3)]Glcβ1- | Steviol |
| Steviosid eD | 787 | 2 | 1 | | Glcβ1- | Glcβ(1-2)6-deoxyGlcβ1- | |
| Iso-Reb B | 803 | 3 | | | H- | Glcβ(1-2)[Glcβ(1-3)]Glcβ1- | Isosteviol |
| Iso-Steviosid e | 803 | 3 | | | Glcβ1- | Glcβ(1-2)Glcβ1- | Isosteviol |
| Reb B | 803 | 3 | | | H- | Glcβ(1-2)[Glcβ(1-3)]Glcβ1- | Steviol |
| Reb G | 803 | 3 | | | Glcβ1- | Glcβ(1-3)Glcβ1- | Steviol |
| Reb-KA | 803 | 3 | | | Glcβ(1-2)Glcβ1- | Glcβ1- | Steviol |
| SG-13 | 803 | 3 | | | Glcβ1- | Glcβ(1-2)Glcβ1- | Isomeric steviol (12α-hydroxy) |
| Steviosid e | 803 | 3 | | | Glcβ1- | Glcβ(1-2)Glcβ1- | Steviol |
| Steviosid e B (SG-15) | 803 | 3 | | | Glcβ(1-3)Glcβ1- | Glcβ1- | Steviol |
| Reb F | 935 | 3 | | 1 | Glcβ1- | Xylβ(1-2)[Glcβ(1-3)]Glcβ1- | Steviol |
| Reb R | 935 | 3 | | 1 | Glcβ1- | Glcβ(1-2)[Glcβ(1-3)]Xylβ1- | Steviol |
| SG-Unk2 | 935 | 3 | | 1 | - | - | Steviol |
| SG-Unk3 | 935 | 3 | | 1 | - | - | Steviol |
| Reb F3 (SG-11) | 935 | 3 | | 1 | Xylβ(1-6)Glcβ1- | Glcβ(1-2)Glcβ1- | Steviol |
| Reb F2 (SG-14) | 935 | 3 | | 1 | Glcβ1- | Glcβ(1-2)[Xylβ(1-3)]Glcβ1- | Steviol |
| Reb C | 949 | 3 | 1 | | Glcβ1- | Rhaα(1-2)[Glcβ(1-3)]Glcβ1- | Steviol |
| Reb C2/Reb S | 949 | 3 | 1 | | Rhaα(1-2)Glcβ1- | Glcβ(1-2)Glcβ1- | Steviol |
| Steviosid e E (SG-9) | 949 | 3 | 1 | | Glcβ1- | 6-DeoxyGlcβ(1-2)[Glcβ(1-3)]Glcβ1- | Steviol |
| Steviosid e E2 | 949 | 3 | 1 | | 6-DeoxyGlcβ 1- | Glcβ(1-2)[Glcβ(1-3)]Glcβ1- | |
| SG-10 | 949 | 3 | 1 | | Glcβ1- | Glcα(1-3)Glcβ(1-2)[Glcβ(1-3])Glcβ1- | Steviol |
| Reb L1 | 949 | 3 | 1 | | H- | Glcβ(1-3)Rhaα(1-2)[Glcβ(1-3)]Glcβ1- | Steviol |
| SG-2 | 949 | 3 | 1 | | Glcβ1- | 6-deoxyGlcβ(1-2)[Glcβ(1-3)]Glcβ1- | Steviol |
| Reb A3 (SG-8) | 965 | 4 (1 Fru | | | Glcβ1- | Glcβ(1-2)[Fruβ(1-3)]Glcβ1- | |
| Iso-Reb A | 965 | 4 | | | Glcβ1- | Glcβ(1-2)[Glcβ(1-3)]Glcβ1- | Isosteviol |
| Reb A | 965 | 4 | | | Glcβ1- | Glcβ(1-2)[Glcβ(1-3)]Glcβ1- | Steviol |
| Reb A2 (SG-7) | 965 | 4 | | | Glcβ1- | Glcβ(1-6)[Glcβ(1 -2)]Glcβ1- | Steviol |
| Reb E | 965 | 4 | | | Glcβ(1-2)Glcβ1- | Glcβ(1-2)Glcβ1- | Steviol |
| Reb H1 | 965 | 4 | | | H- | Glcβ(1-6)Glcβ(1-3)[Glcβ1-3)]Glcβ1- | Steviol |
| Related SvGn#2 | 981 | - | | | | | |
| Related SvGn#5 | 981 | - | | | | | |
| Reb U2 | 1097 | 4 | | 1 | Xylβ(1-2)[Glcβ(1-3)]Glcβ1- | Glcβ(1-2)Glcβ1- | |
| Reb T | 1097 | 4 | | 1 | Xylβ(1-2)Glcβ1- | Glcβ(1-2)[Glcβ(1-3)]Glcβ1- | |
| Reb W | 1097 | 4 | | 1 | Glcβ(1-2)[Araβ(1-3)]Glcβ1- | Glcβ(1-2)Glcβ1- | |
| Reb W2 | 1097 | 4 | | 1 | Araβ(1-2)Glcβ1- | Glcβ(1-2)[Glcβ(1-3)]Glcβ1- | |
| Reb W3 | 1097 | 4 | | 1 | Araβ(1-6)Glcβ1- | Glcβ(1-2)[Glcβ(1-3)]Glcβ1- | |
| Reb U | 1097 | 4 | | 1 | Araα(1-2)-Glcβ1- | Glcβ(1-2)[Glcβ(1-3)]Glcβ1- | Steviol |
| SG-12 | 1111 | 4 | 1 | | Rhaα(1-2)Glcβ1- | Glcβ(1-2)[Glcβ(1-3)]Glcβ1- | Steviol |
| Reb H | 1111 | 4 | 1 | | Glcβ1- | Glcβ(1-3)Rhaα(1-2)[Glcβ(1-3)]Glcβ1- | Steviol |
| Reb J | 1111 | 4 | 1 | | Rhaα(1-2)Glcβ1- | Glcβ(1-2)[Glcβ(1-3)]Glcβ1- | Steviol |
| Reb K | 1111 | 4 | 1 | | Glcβ(1-2)Glcβ1- | Rhaα(1-2)[Glcβ(1-3)]Glcβ1- | Steviol |
| Reb K2 | 1111 | 4 | 1 | | Glcβ(1-6)Glcβ1- | Rhaα(1-2)[Glcβ(1-3)]Glcβ1- | Steviol |
| SG-Unk4 | 1111 | 4 | 1 | | – | – | Steviol |
| SG-Unk5 | 1111 | 4 | 1 | | – | – | Steviol |
| Reb D | 1127 | 5 | | | Glcβ(1-2)Glcβ1- | Glcβ(1-2)[Glcβ(1-3)]Glcβ1- | Steviol |
| Reb I | 1127 | 5 | | | Glcβ(1-3)Glcβ1- | Glcβ(1-2)[Glcβ(1-3)]Glcβ1- | Steviol |
| Reb L | 1127 | 5 | | | Glcβ1- | Glcβ(1-6)Glcβ(1-2)[Glcβ(1-3)]Glcβ1- | Steviol |
| Reb 13 | 1127 | 5 | | | [Glcβ(1-2) Glcβ(1-6)] Glcβ1- | Glcβ(1-2)Glcβ1- | |
| SG-Unk6 | 1127 | 5 | | | - | - | Steviol |
| Reb Q (SG-5) | 1127 | 5 | | | Glcβ1- | Glcα(1-4)Glcβ(1-2)[Glcβ(1-3)]Glcβ1- | Steviol |
| Reb 12 (SG-6) | 1127 | 5 | | | Glcβ1- | Glcα(1-3)Glcβ1-2[Glcβ1-3)]Glcβ1- | Steviol |
| Reb Q2 | 1127 | 5 | | | Glcα(1-2)Glcα(1-4)Glcβ1- | Glcβ(1-2)Glcβ1- | |
| Reb Q3 | 1127 | 5 | | | Glcβ1- | Glcα(1-4)Glcβ(1-3)[Glcβ(1-2)]Glcβ1- | |
| Reb T1 | 1127 | 5 (1 Gal) | | | Galβ(1-2)Glcβ1- | Glcβ(1-2)[Glcβ(1-3)]Glcβ1- | |
| Related SvGn#4 | 1127 | – | | | | | |
| Reb V2 | 1259 | 5 | | 1 | Xylβ(1-2)[Glcβ(1-3)]-Glcβ1- | Glcβ(1-2)[Glcβ(1-3)]Glcβ1- | Steviol |
| Reb V | 1259 | 5 | | 1 | Glcβ(1-2)[Glcβ(1-3)]Glcβ1- | Xylβ(1-2)[Glcβ(1-3)]-Glcβ1- | |
| Reb Y | 1259 | 5 | | 1 | Glcβ(1-2)[Araβ(1-3)]Glcβ1- | Glcβ(1-2)[Glcβ(1-3)]Glcβ1- | |
| Reb N | 1273 | 5 | 1 | | Rhaα(1-2)[Glcβ(1-3)]Glcβ1- | Glcβ(1-2)[Glcβ(1-3)]Glcβ1- | Steviol |
| Reb M | 1289 | 6 | | | Glcβ(1-2)[Glcβ(1-3)]Glcβ1- | Glcβ(1-2)[Glcβ(1-3)]Glcβ1- | Steviol |
| 15α-OHReb M | 1305 | 6 | | | Glcβ1-2(Glcβ1-3)Glcβ1- | Glcβ(1-2)[Glcβ1-3]Glcβ1- | 15α-Hydroxy-steviol |
| Reb O | 1435 | 6 | 1 | | Glcβ(1-3)Rhaα(1-2)[Glcβ(1-3)]Glcβ1- | Glcβ(1-2)[Glcβ(1-3)]Glcβ1- | Steviol |
| Reb O2 | 1435 | 6 | 1 | | Glcβ(1-4)Rhaα(1-2)[Glcβ(1-3)]Glcβ1- | Glcβ(1-2)[Glcβ(1-3)]Glcβ1- | |

Legend: SG-1 to 16: SGs without a specific name; SG-Unk1-6: SGs without detailed structural proof; Glc: Glucose; Rha: Rhamnose; Xyl: Xylose; Ara: Arabinose.

As described above, the present application provides compositions that may include one or more glycosylated NHIS formed from a reaction mixture containing a NHIS. As used herein, a glycosylated NHIS may also be referred to as a derivative of NHIS.

Generally, the glycosylated NHIS of the present application is prepared as follows: (i) dissolving a sugar-donor material in water to form a liquefied sugar-donor material; (ii) adding a starting NHIS composition to liquefied sugar-donor material to obtain a mixture; and (iii) adding an effective amount of an enzyme to the mixture to form a reaction mixture, wherein the enzyme catalyzes the transfer of sugar moieties from the sugar-donor material to the SG in the starting NHIS composition; and (iv) incubating the reaction mixture at a desired temperature for a desired length of reaction time to glycosylate the NHIS in the starting NHIS composition with sugar moieties present in the sugar-donor molecule.

After achieving a desired ratio of glycosylated NHIS and residual NHIS (i.e., unreacted NHIS) contents, the reaction mixture can be heated to a sufficient temperature for a sufficient amount of time to inactivate the enzyme. In some embodiments, the enzyme is removed by filtration in lieu of inactivation. In other embodiments, the enzyme is removed by filtration following inactivation. In some embodiments the sugar is glucose and the sugar donor is a glucose donor. In some embodiments, the glucose donor is starch. In some embodiments the resulting solution comprising glycosylated NHIS, residual NHIS and dextrin is decolorized.

In some embodiments the resulting solution of glycosylated NHIS, including residual NHIS and dextrin is dried. In some embodiments, the drying is by spray drying. In some embodiments, step (i) comprises the substeps of (a) mixing a glucose-donor material with a desired amount of water to form a suspension, (b) adding a desired amount of enzyme to the suspension and (c) incubate the suspension at a desired temperature for a desired time to form liquefied glucose-donor material. Starch can be a suitable substitute for dextrin(s) and/or dextrin(s) can be obtained by the hydrolysis of starch. The unreacted NHIS, with or without dextrins, can be separated from glycosylated NHIS if necessary.

In some embodiments, the composition of the present application comprises one or more glycosylated NHIS in an amount of 0.001-99 wt %, 0.001-75 wt %, 0.001-50 wt%, 0.001-25 wt%, 0.001-10 wt %, 0.001-5 wt %, 0.001-2 wt %, 0.001-1 wt %, 0.001-0.1 wt %, 0.001-0.01 wt %, 0.01-99 wt %, 0.01-75 wt %, 0.01-50 wt%, 0.01-25 wt%., 0.01-10 wt %, 0.01-5 wt %, 0.01-2 wt %, 0.01-1 wt %, 0.1-99 wt %, 0.1-75 wt %, 0.1 wt-50 wt%, 0.1-25 wt%, 0.1-10 wt %, 0.1-5 wt %, 0.1-2 wt %, 0.1-1 wt %, 0. 1-0.5 wt %, 1-99 wt %, 1-75 wt %, 1-50 wt%, 1-25 wt%, 1-10 wt %, 1-5 wt %, 5-99 wt %, 5-75 wt %, 5-50 wt%, 5-25 wt%, 5-10 wt %, 10-99 wt %, 10-75 wt %, 10-50 wt%, 10-25 wt%, 10-15 wt %, 20-99 wt %, 20-75 wt %, 20-50 wt%, 30-99 wt %, 30-75 wt %, 30-50 wt%, 40-99 wt %, 40-75 wt %, 40-50 wt%, 50-99 wt %, 50-75 wt %, 60-99 wt %, 60-75 wt %, 70-99 wt %, 70-75 wt %, 80-99 wt %, 80-90 wt %, 90-99 wt% of the composition.

In some embodiments, the glycosylated NHIS is a mono-glycosylated, di-glycosylated, tri-glycosylated, tetra-glycosylated, or penta-glycosylated glycosylation product of a NHIS.

### (I) Glycosylation Reaction

The glycosylated NHIS of the present application can be prepared by an enzyme-mediated or non-enzyme-mediated process in which one or more sugar residues are transferred from one or more sugar donors to a substrate to produce a glycosylated NHIS product, such as a GSG product. This process is referred to hereinafter as a glycosylation reaction.

In some embodiments, the glycosylated NHIS product is a GSG product. The GSGs of the present application can also be prepared by a enzyme-mediated or nonenzyme-mediated process in which one or more sugar residues are removed from a GSG substrate to produce a new GSG product. This process is referred to hereinafter as a conversion reaction. The substrate of a conversion reaction can be, for example, a GRA, GRB, GRC, GRD, GRE, GRF, GRI, GRM, GRN, GRO, glycosylated steviolmonoside, glycosylated steviolbioside, glycosylated dulcoside, glycosylated rubusoside or glycosylated stevioside.

In some embodiments, the glycosylated NHIS products described in the present application, such as the GSG products, are formed by an exogenous glycosylation reaction in the present of a glycosyltransferase.

As used herein, a “glycosyltransferase” refers to an enzyme that catalyzes the formation of a glycosidic linkage to form a glycoside. A glycoside is any molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. The sugar group is known as the glycone and the non-sugar group is known as the aglycone. The glycone can be part of a single sugar group (monosaccharide) or several sugar groups (oligosaccharide). A glycosyltransferase according to the present application further embraces “glycosyltransferase variants” engineered for enhanced activities.

Glycosyltransferases utilize “activated” sugar phosphates as glycosyl donors, and catalyze glycosyl group transfer to an acceptor molecule comprising a nucleophilic group, usually an alcohol. A retaining glycosyltransferases is one which transfers a sugar residue with the retention of anomeric configuration. Retaining glycosyltransferase enzymes retain the stereochemistry of the donor glycosidic linkage after transfer to an acceptor molecule. An inverting glycosyltransferase, on the other hand, is one which transfers a sugar residue with the inversion of anomeric configuration. Glycosyltransferases are classified based on amino acid sequence similarities. Glycosyltransferases are classified by the Nomenclature Committee of the International Union of Biochemistry and Molecular Biology (NC-IUBMB) in the enzyme class of EC 2.4.1 on the basis of the reaction catalyzed and the specificity.

Glycosyltransferases can utilize a range of donor substrates. Based on the type of donor sugar transferred, these enzymes are grouped into families based on sequence similarities. Exemplary glycosyltransferases include glucanotransferases, N-acetylglucosaminyltransferases, N-acetylgalactosaminyltransferases, fucosyltransferases, mannosyltransferases, galactosyltransferases, sialyltransferases, galactosyltransferases, fucosyltransferase, Leloir glycosyltransferases, non-Leloir glycosyltransferases, and other glycosyltransferases in the enzyme class of EC 2.4.1. The Carbohydrate-Active Enzymes database (CAZy) provides a continuously updated list of the glycosyltransferase families.

In some embodiments, the glycosylated NHIS products are formed from a reaction mixture comprising an exogenous glycosyltransferase classified as an EC 2.4.1 enzyme, including but not limited to members selected from the group consisting of cyclomaltodextrin glucanotransferase (CGTase; EC 2.4.1.19), amylosucrase (EC 2.4.1.4), dextransucrase (EC 2.4.1.5), amylomaltase, sucrose:sucrose fructosyltransferase (EC 2.4.1.99), 4-α-glucanotransferase (EC 2.4.1.25), lactose synthase (EC 2.4.1.22), sucrose-1,6-α-glucan 3(6)-α-glucosyltransferase, maltose synthase (EC 2.4.1.139), alternasucrase (EC 2.4.1.140), including variants thereof.

Cyclomaltodextrin glucanotransferase, also known as CGTase, is an enzyme assigned with enzyme classification number EC 2.4.1.19, which is capable of catalyzing the hydrolysis and formation of (1→4)-α-D-lucosidic bonds, and in particular the formation of cyclic maltodextrins from polysaccharides as well as the disproportionation of linear oligosaccharides.

Dextransucrase is an enzyme assigned with enzyme classification number EC 2.4.1.5, and is also known as sucrose 6-glucosyltransferase, SGE, CEP, sucrose-1,6-α-glucan glucosyltransferase or sucrose: 1,6-α-D-glucan 6-α-D-glucosyltransferase. Dextransucrases are capable of catalyzing the reaction: sucrose + [(1→6)-α-D-glucosyl]n = D-fructose + [(1→6)-α-D-glucosyl]n+1. In addition, a glucosyltransferase (DsrE) from Leuconostoc mesenteroides, NRRL B-1299 has a second catalytic domain (“CD2”) capable of adding alpha-1,2 branching to dextrans (U.S. Pat. Nos. 7,439,049 and 5,141,858; U.S. Pat. Appl. Publ. No. 2009-0123448; Bozonnet et al., J. Bacteria 184:5753-5761, 2002).

Glycosyltransferases and other glycosylating enzymes for use in the present application may be derived from any source and may be used in a purified form, in an enriched concentrate or as a crude enzyme preparation.

In some embodiments, the glycosylation reaction is carried out by glycosylating an aglycone or glycoside substrate using e.g., a nucleotide sugar donor (e.g., sugar mono- or diphosphonucleotide) or “Leloir donor” in conjunction with a “Leloir glycosyltransferase” (after Nobel prize winner, Luis Leloir) that catalyzes the transfer of a monosaccharide unit from the nucleotide-sugar (“glycosyl donor’) to a “glycosyl acceptor”, typically a hydroxyl group in an aglycone or glycoside substrate.

Accordingly, in some embodiments the glycosylated NHIS products of the present application are formed from a reaction mixture comprising a nucleotide sugar.

In certain embodiments, the glycosylation reactions may involve the use of a specific Leloir glycosyltransferase in conjunction with a wide range of sugar nucleotides donors, including e.g., UDP-glucose, GDP-glucose, ADP-glucose, CDP-glucose, TDP-glucose or IDT-glucose in combination with a glucose-dependent glycosyltransferase (GDP-glycosyltransferases; GGTs), ADP-glucose-dependent glycosyltransferase (ADP-glycosyltransferases; AGTs), CDP-glucose-dependent glycosyltransferase (CDP-glycosyltransferases; CGTs), TDP-glucose-dependent glycosyltransferase (TDP-glycosyltransferases;TGTs) or IDP-glucose-dependent glycosyltransferase (IDP-glycosyltransferases; IGTs), respectively.

In particular embodiments, the exogenous glycosylation reaction is carried out using an exogenous Leloir-type UDP-glycosyltransferase enzyme of the classification EC 2.4.1.17, which catalyzes the transfer of glucose from UDP-α-D-glucuronate (also known as UDP-glucose) to an acceptor, releasing UDP and forming acceptor β-D-glucuronoside. In some embodiments, the glycosyltransferases include, but are not limited to, enzymes classified in the GT1 family. In certain preferred embodiment, the glycosylation reaction is catalyzed by an exogenous UDP-glucose-dependent glycosyltransferase. In some embodiments, the glycosylation reaction is catalyzed by a glycosyltransferase capable of transferring a non-glucose monosaccharide, such as fructose, galactose, ribose, arabinose, xylose, mannose, psicose, fucose and rhamnose, and derivative thereof, to the recipient.

U.S. Pat. No. 9,567,619 describes several UDP-dependent glycosyltransferases that can be used to transfer monosaccharides to rubusoside, including UGT76G1 UDP glycosyltransferase, HV1 UDP-glycosyltransferase, and EUGT11, a UDP glycosyltransferase-sucrose synthase fusion enzyme. The EUGT11 fusion enzyme contains a uridine diphospho glycosyltransferase domain coupled to a sucrose synthase domain and can exhibit 1,2-β glycosidic linkage and 1,6-β glycosidic linkage enzymatic activities, as well as sucrose synthase activity. Of the foregoing enzymes, UGT76G1 UDP glycosyltransferase contains a 1,3-O-glucose glycosylation activity which can transfer a second glucose moiety to the C-3′ of 13-O-glucose of rubusoside to produce rebaudioside G (“Reb G”); HV1 UDP-glycosyltransferase contains a 1,2-O-glucose glycosylation activity which can transfer a second glucoside moiety to the C-2′ of 19-O-glucose of rubusoside to produce rebaudioside KA (“Reb KA”); and the EUGT11 fusion enzyme contains a 1,2-O-glucose glycosylation activity which transfers a second glucose moiety to the C-2′ of 19-O-glucose of rubusoside to produce rebaudioside KA or transfer a second glucose moiety to the C-2′ of 13-O-glucose of rubusoside to produce stevioside. In addition, HV1 and EUGT11 can transfer a second sugar moiety to the C-2′ of 19-O-glucose of rebaudioside G to produce rebaudioside V (“Reb V”) and can additionally transfer a second glucose moiety to the C-2′ of 13-O-glucose of rebaudioside KA to produce rebaudioside E (“Reb E”). Furthermore, when used singly or in combination, these enzymes can be used to generate a variety of steviol glycosides known to be present in Stevia rebaudiana, including rebaudioside D (“Reb D”) and rebaudioside M (“Reb M”).

In some embodiments, monosaccharides that can be transferred to a saccharide or monosaccharide acceptor include, but are not limited to glucose, fructose, galactose, ribose, arabinose, xylose, mannose, psicose, fucose and rhamnose, and derivative thereof, as well as acidic sugars, such as sialic acid, glucuronic acid and galacturonic acid.

In some embodiments, glycosylation of SGs is driven by an exogenous glycosyl hydrolase (GH). GHs normally cleave a glycosidic bond. However, they can be used to form glycosides by selecting conditions that favor synthesis via reverse hydrolysis. Reverse hydrolysis is frequently applied e.g., in the synthesis of aliphatic alkylmonoglucosides.

Glycosyl hydrolases have a wide range of donor substrates employing usually monosaccharides, oligosaccharides or/and engineered substrates (i.e., substrates carrying various functional groups). They often display activity towards a large variety of carbohydrate and non-carbohydrate acceptors. Glycosidases usually catalyze the hydrolysis of glycosidic linkages with either retention or inversion of stereochemical configuration in the product.

In some embodiments, the GRGs of the present application are formed from a reaction mixture comprising an exogenous glycosyl hydrolase, classified as an EC 3.2.1 enzyme, including but not limited to alpha-glucosidase, beta-glucosidase and beta-fructofuranosidase.

Exemplary glycosyl hydrolases for use in the present application include, but are not limited to α-amylases (EC 3.2.1.1), α-glucosidases (EC 3.2.1.20), β-glucosidases (EC 3.2.1.21), α-galactosidases (EC 3.2.1.22), β-galactosidases (EC 3.2.1.23), α-mannosidase (EC 3.2.1.24), β-mannosidase (EC 3.2.1.25), β-fructofuranosidase (EC 3.2.1.26), amylo-1,6-glucosidases (EC 3.2.1.33), β-D-fucosidases (EC 3.2.1.38), α-L-rhamnosidases (EC 3.21.40), glucan 1,6-α-glucosidases (EC 3.2.70), and variants thereof.

In some embodiments, the GRGs of the present application are formed using a class of glycoside hydrolases or glycosyltransferases known as “transglycosylases.” As used herein, the term “transglycosylase” and “transglycosidase” (TG) are used interchangeably with reference to a glycoside hydrolase (GH) or glycosyltransferase (GT) enzyme capable of transferring a monosaccharide moiety from one molecule to another. Thus, a GH can catalyze the formation of a new glycosidic bond either by transglycosylation or by reverse hydrolysis (i.e., condensation).

The acceptor for transglycosylase reaction acceptor can be saccharide acceptor or a monosaccharide acceptor. Thus, a transglycosidase can transfer a monosaccharide moiety to a diverse set of aglycones, including e.g., monosaccharide acceptors, such as aromatic and aliphatic alcohols. Transglycosidases can transfer a wide variety of monosaccharides (D- or L-configurations) to saccharide acceptors, including glycosides, as well as monosaccharide acceptors, including a wide variety of flavonoid aglycones, such as naringenin, quercetin, hesperetin.

Monosaccharides that can be transferred to a saccharide or monosaccharide acceptor include, but are not limited to glucose, fructose, galactose, ribose, arabinose, xylose, mannose, psicose, fucose and rhamnose, and derivative thereof, as well as acidic sugars, such as sialic acid, glucuronic acid and galacturonic acid. The term “transglucosidase” is used when the monosaccharide moiety is a glucose moiety.

Transglycosidases include GHs or GTs from the enzyme classes of EC 3.2.1 or 2.4.1, respectively. In spite of the inclusion of certain glycosyltransferases as transglycosidases, TGs are classified into various GH families on the basis of sequence similarity. A large number of retaining glycosidases catalyze both hydrolysis and transglycosylation reactions. In particular, these enzymes catalyze the intra- or intermolecular substitution of the anomeric position of a glycoside. Under kinetically controlled reactions, retaining glycosidases can be used to form glycosidic linkages using a glycosyl donor activated by a good anomeric leaving group (e.g., nitrophenyl glycoside). In contrast, thermodynamically controlled reverse hydrolysis uses high concentrations of free sugars.

Transglycosidases corresponding to any of the GH families with notable transglycosylase activity may be used in the present application, and may include the use of e.g., members of the GH2 family, including LacZ β-galactosidase, which converts lactose to allolactose; GH13 family, which includes cyclodextran glucanotransferases that convert linear amylose to cyclodextrins, glycogen debranching enzyme, which transfers three glucose residues from the four-residue glycogen branch to a nearby branch, and trehalose synthase, which catalyzes the interconversion of maltose and trehalose; GH16 family, including xyloglucan endotransglycosylases, which cuts and rejoins xyloglucan chains in the plant cell wall; GH31, for example, α-transglucosidases, which catalyze the transfer of individual glucosyl residues between α-(1→4)-glucans; GH70 family, for example, glucansucrases, which catalyze the synthesis of high molecular weight glucans, from sucrose; GH77 family, for examples amylomaltase, which catalyzes the synthesis of maltodextrins from maltose; and the GH23, GH102, GH103, and GH104 families, which include lytic transglycosylases that convert peptidoglycan to 1,6-anhydrosugars.

In one embodiment, the glycosyltransferase is a transglucosylase from the glycoside hydrolase 70 (GH70) family. GH70 enzymes are transglucosylases produced by lactic acid bacteria from, e.g., Streptococcus, Leuconostoc, Weisella or Lactobacillus genera. Together with the families GH13 and GH77 enzymes, they form the clan GH-H. Most of the enzymes classified in this family use sucrose as the D-glucopyranosyl donor to synthesize α-D-glucans of high molecular mass (>106 Da) with the concomitant release of D-fructose. They are also referred to as glucosyltransferases or glucansucrases.

A wide range of α-D-glucans, varying in size, structure, degree of branching and spatial arrangements can thus be produced by GH70 family members. For example, GH70 glucansucrases can transfer D-glucosyl units from sucrose onto hydroxyl acceptor groups. Glucansucrases catalyze the formation of linear as well as branched α-D-glucan chains with various types of glycosidic linkages, namely α-1,2; α-1,3; α-1,4; and/or α-1,6.

In addition, sucrose analogues such as α-D-glucopyranosyl fluoride, p-nitrophenyl α-D-glucopyranoside, α-D-glucopyranosyl α-L-sorofuranoside and lactulosucrose can be utilized as D-glucopyranosyl donors. A large variety of acceptors may be recognized by glucansucrases, including carbohydrates, alcohols, polyols or flavonoids to yield oligosaccharides or gluco-conjugates.

Exemplary glucansucrases for use in the present application include e.g., dextransucrase (sucrose:1,6-α-D-glucosyltransferase; EC 2.4.1.5), alternansucrase (sucrose:1,6(1,3)-α-D-glucan-6(3)-α-D-glucosyltransferase, EC 2.4.1.140), mutansucrase (sucrose:1,3-αD-glucan-3-α-D-glucosyltransferase; EC 2.4.1.125), and reuteransucrase (sucrose:1,4(6-α-D-glucan-4(6)-αD-glucosyltransferase; EC 2.4.1.-). The structure of the resultant glucosylated product is dependent upon the enzyme specificity.

In some embodiments, a fructosyltransferase may be used to catalyze the transfer of one or more fructose units, optionally comprising terminal glucose, of the following sequence: (Fru)n-Glc consisting of one or more of: β 2,1, β 2,6, α 1,2 and β-1,2 glycosidic bonds, wherein n typically is 3-10. Variants include Inulin type β-1,2 and Levan type β-2,6 linkages between fructosyl units in the main chain. Exemplary fructosytransferase for use in the present application include e.g., β-fructofuranosidase (EC 3.2.1.26), inulosucrase (EC 2.4.1.9) levansucrase (EC 2.4.1.10), or endoinulinase.

In some embodiments, a galactosyltransferase or β-galactosidase may be used to catalyze the transfer of multiple saccharide units, in which one of the units is a terminal glucose and the remaining units are galactose and disaccharides comprising two units of galactose. In certain embodiments, the resulting structure includes a mixture of galactopyranosyl oligomers (DP=3-8) linked mostly by β-(1,4) or β-(1,6) bonds, although low proportions of β-(1,2) or β-(1,3) linkages may also be present. Terminal glucosyl residues are linked by β-(1,4) bonds to galactosyl units. These structures may be synthesized by the reverse action of β-galactosidases (EC 3.2.1.23) on lactose at relatively high concentrations of lactose.

In some embodiments, the transglycosidase is an enzyme having trans-fucosidase, trans-sialidase, trans-lacto-N-biosidase and/or trans-N-acetyllactosaminidase activity.

In some embodiments, the glycosylation reactions may utilize a combination of any of glycosyltransferases described herein in combination with any one of the glycosyl hydrolases or transglycosidases described herein. In these reactions, the transglycosylase and the glycosyl hydrolase or transglycosidase may be present in a range of ratios (w/w), wherein the transglycosylase/glycosyl hydrolase ratio (w/w) ranges from 100:1, 80:1, 60:1, 40:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:80, 1:100, or any ratio derived from any two of the aforementioned integers.

In some embodiments, the sugar donor in the glycosylation reaction is a glucose-based donor. Examples of glucose-based donors include, but are not limited to, glucose, dextrin, and maltodextrin.

In some embodiments, the sugar donor in the glycosylation reaction is a non-glucose-based sugar. Examples of non-glucose-based sugars include, but are not limited to, arabinose, fructose, galactose, lactose, mannose, rhamnose and xylose.

In some embodiments, a glycosylation reaction is performed with a combination of different sugars a sugar donor.

In some embodiments, multiple rounds of glycosylation reaction are performed with a different sugar donor in each round.

In some embodiments, the substrate of the glycosylation reaction is a SG, such as RA, RB, RC, RD, RE, RF, RI, RM, RN, RO, steviolmonoside, steviolbioside, dulcoside A, dulcoside B, rubusoside and stevioside.

In some embodiment, the substrate of the glycosylation reaction is a GSG, such as GRA, GRB, GRC, GRD, GRE, GRF, GRI, GRM, GRN, GRO, glycosylated steviolmonoside, glycosylated steviolbioside, glycosylated dulcoside, glycosylated rubusoside and glycosylated stevioside.

In some embodiments, the glycosylation reaction is performed with substrate-to-sugar donor weight ratio in the range of 99:1 to 1:99, 90:1 to 1:99, 80:1 to 1:99, 70:1 to 1:99, 60:1 to 1:99, 50:1 to 1:99, 40:1 to 1:99, 30:1 to 1:99, 20:1 to 1:99, 10:1 to 1:99, 1:1 to 1:99, 99:1 to 1:90, 90:1 to 1:90, 80:1 to 1:90, 70:1 to 1:90, 60:1 to 1:90, 50:1 to 1:90, 40:1 to 1:90, 30:1 to 1:90, 20:1 to 1:90, 10:1 to 1:90, 1:1 to 1:90, 99:1 to 1:60, 90:1 to 1:60, 80:1 to 1:60, 70:1 to 1:60, 60:1 to 1:60, 50:1 to 1:60, 40:1 to 1:60, 30:1 to 1:60, 20:1 to 1:60, 10:1 to 1:60, 1:1 to 1:60, 99:1 to 1:30, 90:1 to 1:30, 80:1 to 1:30, 70:1 to 1:30, 60:1 to 1:30, 50:1 to 1:30, 40:1 to 1:30, 30:1 to 1:30, 20:1 to 1:30, 10:1 to 1:30, 1:1 to 1:30, 99:1 to 1:10, 90:1 to 1:10, 80:1 to 1:10, 70:1 to 1:10, 60:1 to 1:10, 50:1 to 1:10, 40:1 to 1:10, 30:1 to 1:10, 20:1 to 1:10, 10:1 to 1:10, 1:1 to 1:10.

The glycosylating enzyme may be dissolved in the reaction mixture or immobilized on a solid support which is contacted with the reaction mixture. If the enzyme is immobilized, it may be attached to an inert carrier. Suitable carrier materials are known in the art. Examples for suitable carrier materials are clays, clay minerals such as kaolinite, diatomeceous earth, perlite, silica, alumina, sodium carbonate, calcium carbonate, cellulose powder, anion exchanger materials, synthetic polymers, such as polystyrene, acrylic resins, phenol formaldehyde resins, polyurethanes and polyolefins, such as polyethylene and polypropylene. For preparing carrier-bound enzymes the carrier materials usually are used in the form of fine powders, wherein porous forms are preferred. The particle size of the carrier material usually does not exceed 5 mm, in particular 2 mm. Further, suitable carrier materials are calcium alginate and carrageenan. Enzymes may directly be linked by glutaraldehyde. A wide range of immobilization methods are known in the art. Ratio of reactants can be adjusted based on the desired performance of the final product. The temperature of the glycosylation reaction can be in the range of 1-100° C., preferably 40-80° C., more preferably 50-70° C.

In certain embodiments, the GSG used in the present application are prepared as follows: (i) mixing a starting SG composition (e.g., a rubusoside) with a sugar-donor material to obtain a mixture; and (ii) adding an effective amount of an enzyme to the mixture to form a reaction mixture, where the enzyme catalyzes the transfer of sugar moieties from the sugar-donor material to the SG molecules in the starting SG composition; and (iii) incubating the reaction mixture at a desired temperature for a desired length of reaction time to glycosylate the SG molecules with sugar moieties present in the sugar-donor molecule to generate GSG. In some embodiments, after achieving a desired ratio of GSG to residual SG contents, the reaction mixture can be heated to a sufficient temperature for a sufficient amount of time to inactivate the enzyme. In some embodiments, the enzyme is removed by filtration in lieu of inactivation. In other embodiments, the enzyme is removed by filtration following inactivation, resulting a solution comprising GSG, residual SG from the starting SG composition and residual sugar donor. In some embodiments the resulting solution comprising GSG, residual SG and residue sugar donor is decolorized.

Examples of sugar donors include, but are not limited to, dextrin, maltodextin, glucose, fructose, galactose, lactose, mannose, fruit juice, vegetable juice and honey.

In some embodiments, the GSG used in the present application are prepared as follows: (i) dissolving a glucose-donor material in water to form a liquefied glucose-donor material; (ii) adding a starting SG composition to liquefied glucose-donor material to obtain a mixture; and (iii) adding an effective amount of an enzyme to the mixture to form a reaction mixture, wherein the enzyme catalyzes the transfer of glucose moieties from the glucose-donor material to the SG molecules in the starting SG composition; and (iv) incubating the reaction mixture at a desired temperature for a desired length of reaction time to glycosylate the SG molecules with glucose moieties present in the glucose-donor molecule. In some embodiments, after achieving a desired ratio of GSG and SG contents, the reaction mixture is heated to a sufficient temperature for a sufficient amount of time to inactivate the enzyme. In some embodiments, the enzyme is removed by filtration in lieu of inactivation. In other embodiments, the enzyme is removed by filtration following inactivation. In some embodiments the resulting solution comprising GSGs, residual SGs and dextrin is decolorized. In certain embodiments the resulting solution of GSGs, including residual SGs and dextrin is dried. In some embodiments, the drying is by spray drying. In some embodiments, step (i) comprises the substeps of (a) mixing a glucose-donor material with a desired amount of water to form a suspension, (b) adding a desired amount of enzyme to the suspension and (c) incubate the suspension at a desired temperature for a desired time to form liquefied glucose-donor material. Starch can be a suitable substitute for dextrin(s) and/or dextrin(s) can be obtained by the hydrolysis of starch.

The enzymatically catalyzed reaction can be carried out batch wise, semi-batch wise or continuously. Reactants can be supplied at the start of reaction or can be supplied subsequently, either semi-continuously or continuously. The catalytic amount of glycosidase or glycosyltransferase required for the method of the invention depends on the reaction conditions, such as temperature, solvents and amount of substrate.

The reaction can be performed in aqueous media such as buffer. A buffer adjusts the pH of the reaction mixture to a value suitable for effective enzymatic catalysis. Typically the pH is in the range of about pH 4 to about pH 9, for example, of about pH 5 to about pH 7. Suitable buffers comprise, but are not limited to, sodium acetate, tris(hydroxymethyl) aminomethane (“Tris”) and phosphate buffers.

Optionally, the reaction may take place in the presence of a solvent mixture of water and a water miscible organic solvent at a weight ratio of water to organic solvent of from 0.1: 1 to 9:1, for example, from 1:1 to 3:1. The organic solvent is not primary or secondary alcohol and, accordingly, is non-reactive towards the polysaccharide. Suitable organic solvents comprise alkanones, alkylnitriles, tertiary alcohols and cyclic ethers, and mixtures thereof, for example, acetone, acetonitrile, t-pentanol, t-butanol, 1,4-dioxane and tetrahydrofuran, and mixtures thereof. Generally, the use of organic solvents is not preferred.

The final product of the glycosylation reaction, such as glycosylated rubusoside and glycosylated stevioside, may be further purified to remove residual sugar donor, such as maltodextrin.

In some embodiments, a GSG, such as glycosylated rubusoside is subjected to enzyme treatment (e.g., α-amylase treatment) to produce a GSG with reduced level of glycosylation (e.g., GSG with shortened side chains at the glycosylation sites) compared to the pre-treatment GSG.

In some embodiments, a GSG, such as glycosylated rubusoside, is subjected to another glycosylation reaction to produce GSGs with increased level of glycosylation (e.g., elongated side chains at the glycosylation sites) compared to the pre-treatment GSG.

### (II) Conversion Reactions

### (A) Enzyme-Mediated (or Enzymatic) Conversion

In some embodiments, the glycosylated NHIS, such as GSGs, of the present application are formed by an exogenous conversion reaction in the present of a glycosyl hydrolase (GH), which cleaves a glycosidic bond and is thus capable of converting a GSG, such as glycosylated stevioside to another GSG, such as glycosylated rubusoside, by removing a glucose at the C-13 position of the stevioside.

### (B) Non-Enzyme-Mediated (Non-Enzymatic) Conversion

In some embodiments, the glycosylated NHIS, such as GSGs, of the present application are formed by non-enzymatic hydrolysis. The non-enzymatic hydrolysis can be carried out under alkaline or acid conditions. Table C shows an exemplary list of hydrolysis products from natural diterpene glycoside.

In some embodiments, a GSG of the present application are produced by converting an original GSG into another GSG or GSGs by alkaline or acid hydrolysis.

In some embodiments, glycosylated steviolbioside of the present application is produced from glycosylated stevioside. For examples, glycosylated stevioside can be hydrolyzed to remove a glucose unit from the glycoside chain on the C19 carbon of glycosylated stevioside, which converts glycosylated steviolside to glycosylated steviolbioside.

In some embodiments, glycosylated steviolmonoside of the present application is produced from glycosylated rubusoside. For example, glycosylated rubusoside, can be hydrolyzed to remove a glucose unit from the glycoside chain on the C19 carbon of glycosylated rubusoside, which converts glycosylated rubusoside to glycosylated steviolmonoside.

In some embodiments, glycosylated dulcoside B of the present application is produced from GRC. For example, GRC can be hydrolyzed to remove a glucose unit from the glycoside chain on the C19 carbon of GRC, which converts GRC to glycosylated dulcoside B.

Table C. Compounds reported as degradation products from natural diterpene glycosides by alkaline or acid conditions.

| Common Name | Oligosaccharide Moieties | | AS | Chemical Formula | Starting Material |
|---|---|---|---|---|---|
| | C-13 | C-19 | | | |
| - | Xylβ(1-2)Glcβ₁- | - | I | C₃₁H₄₈O₁₂ | a |
| Dulcoside A₁ | Rhaα(1-2)Glcβ₁- | - | I | C₃₂H₅₀O₁₂ | Dulcoside A |
| Rebaudioside G₁ | Glcβ(1-3)Glcβ₁- | - | I | C₃₂H₅₀O₁₃ | Rebaudioside G |
| Rebaudioside F₁ | Xylβ(1-2)[Glcβ(1-3)]Glcβ₁- | - | I | C₃₇H₅₈O₁₇ | Rebaudioside F |
| Rebaudioside R₁ | Glc(1-2)[Glcβ(1-3)]Xylβ1- | - | I | C₃₇H₅₈O₁₇ | Rebaudioside R |
| Rebaudioside Z₁ | Glcβ(1-6)[Glcβ(1-2)]Glcβ₁- | – | I | C₃₈H₆₀O₁₈ | Rebaudioside Z |
| – | Glcβ(1-2)[Glcβ(1-3)]Glcβ₁- | | II | C₃₈H₆₀O₁₈ | – |
| – | 6-deoxyGlcβ(1-2)[Glcβ(1-3)]Glcβ₁- | – | I | C₃₈H₆₀O₁₇ | b |
| Rebaudioside H₁ | Glcβ(1-6)Glcβ(1-3)[Glcβ(1-3)]Glcβ₁- | – | I | C₄₄H₇₀O₂₃ | Rebaudioside H |
| Rebaudioside L₁ | Glcβ(1-3)Rhaα(1-2)[Glcβ(1-3)]Glcβ₁- | – | I | C₄₄H₇₀O₂₃ | Rebaudioside L |
| Isosteviol | – | – | III | C₂₀H₃₀O₃ | Rebaudioside A/Stevioside |
| Endo-steviol | – | – | IV | C₂₀H₃₀O₃ | Rebaudioside A/Stevioside |
| Endo-steviolmonoside | Glcβ₁- | – | IV | C₂₆H₄₀O₈ | Rebaudioside A/Rubusoside |
| Endo-rebaudioside G₁ | Glcβ(1-3)Glcβ₁- | – | IV | C₃₂H₅₀O₁₃ | Rebaudioside A |
| Endo-steviolbioside | Glcβ(1-2)Glcβ₁- | – | IV | C₃₂H₅₀O₁₃ | Rebaudioside A |
| Endo-rubusoside | Glcβ₁- | Glcβ₁- | IV | C₃₂H₅₀O₁₃ | Rebaudioside A/Rubusoside |
| Iso-stevioside/Endo-stevioside | Glcβ(1-2)Glcβ₁- | Glcβ₁- | IV | C₃₈H₆₀O₁₈ | Rebaudioside A |
| Iso-rebaudioside B/Endo-rebaudioside B | Glcβ(1-2)[Glcβ(1-3)]-Glcβ₁- | – | IV | C₃₈H₆₀O₁₈ | Rebaudioside A |
| – | – | Glcβ(1-2)[Glcβ(1-3)]Glcβ₁- | III | C₃₈H₆₀O₁₈ | Rebaudioside M |
| Iso-rebaudioside AEndo-rebaudioside A | Glcβ(1-2)[Glcβ(1-3)]Glcβ₁- | Glcβ₁- | IV | C₄₄H₇₀O₂₃ | Rebaudioside A |
| – | Glcβ(1-2)[Glcβ(1-3)]Glcβ₁- | Glcβ(1-2)[Glcβ(1-3)]Glcβ₁- | IV | C₅₆H₉₀O₃₃ | Rebaudioside M |
| – | Glcβ(1-2)[Glcβ(1-3)]Glcβ₁- | Glcβ(1-2)[Glcβ(1-3)]Glcβ₁- | V | C₅₆H₉₂O₃₄ | Rebaudioside M |

a 13-[(2-O-β-D-xylopyranosyl-β-D-glucopyranosyl-)oxy]ent-kaur-16-en-19-oic acid β-D-glucopyranosyl ester;
b 13-[(2-O-6-deoxy-β-D-glucopyranosyl-3-O-β-D-glucopyranosyl-β-D-glucopyranosyl)oxy]ent-kaur-16-en-19-oic acid β-D-glucopyranosyl ester.

In some embodiments, alkaline hydrolysis of the starting or raw material is preferred for simplicity and economics. Sodium hydroxide is the preferred alkali to use for hydrolysis of GRA, GRC, glycosylated stevioside or glycosylated rubusoside, but potassium hydroxide and other well-known alkali used in food processing can also be used.

In some embodiments, the starting or raw materials can include 50 wt % or greater, 55 wt % or greater, 60 wt % or greater, 65 wt % or greater, 70 wt % or greater, 75 wt % or greater, 80 wt % or greater, 85 wt % or greater, 90 wt % or greater, 95 wt % or greater, or 99 wt % or greater of GRA, GRC, glycosylated stevioside or glycosylated rubusoside.

In some embodiments, a GSG starting material is dissolved in water (preferably potable water) to form a solution, alkali is then added to the solution , and the solution temperature is raised preferably to 85° C. to 95° C., and more preferably to 90° C. to start alkaline hydrolysis. If the alkaline hydrolysis is conducted at temperatures lower than 85° C., the reaction proceeds slowly until the alkali is exhausted. The solution is stirred and is maintained at the selected temperature for a duration that provides the desired concentrations of the hydrolysis products in the solution or until the alkali is exhausted. The preferred duration of alkaline hydrolysis at commercial scale is a minimum 30 minutes; shorter durations typically do not exhaust the amounts of alkali used in commercial production. The final product solution (containing both the unhydrolyzed GSG starting material such as GRA, also referred to as “residual GSG”, and the hydrolysis product such as GRB) is typically very close to pH 7.0, but pH can be adjusted (typically by adding HCl or NaOH).

The product solution produced as described above may be brown in color, has a faint “burnt sugar” smell, and has a weak “caramel” taste. Brown color, burnt sugar smell, and caramel taste can be removed by column chromatography such as an activated charcoal column, a polymer resin adsorption column or with an ion exchange column as the chromatography matrix, binding the caramel components to the be column while letting the steviol glycosides pass through. Depending upon the use of the sweetener or flavoring agent of the present application, the brown color, burnt sugar smell, and caramel taste may be desirable, or unnoticeable, in either case avoiding the need to remove the brown color, burnt sugar smell, and caramel taste.

The alkaline hydrolysis products can be kept in solution as a syrup ready for distribution as a liquid sweetener, or dried for distribution as a dry sweetener. Drying is by spray-drying, lyophilization, oven drying, and other drying processes well-known in the art of sweeteners.

### (III) Glycosylation and Conversion Products

In some embodiments, the GSG of the present application is a GSG composition obtained from a glycosylation reaction or conversion reaction. In some embodiments, the GSG composition comprises GSGs in an individual or total amount that equals to, or is greater than, 20 wt %, 30 wt %, 40 wt %, 50 wt %, 60 wt %, 70 wt %, 80 wt %, 90 wt % or 95 wt % of the GSG composition. The GSGs may be the glycosylation product of a single SG (e.g., RA, RB, RC, RD, RE, RI, RI, RM, RN, RO, RU, STV, STB, STM, DA etc.) with different levels of glycosylation, or the glycosylation product of multiple SGs with different levels of glycosylation.

In some embodiments, the GSG composition further comprises one or more unreacted residual SGs in an individual or total amount that equals to, or is less than 0.01 wt %, 0.1 wt %, 1 wt %, 2 wt %, 5 wt %, 10 wt %, 20 wt %, 30 wt %, 40 wt % or 50 wt % of the GSG composition.

In some embodiments, the GSG composition further comprises unreacted residual dextrins and/or maltodextrin in an individual or total amount that equals to, or is less than 0.01 wt %, 0.1 wt %, 1 wt %, 2 wt %, 5 wt %, 10 wt %, 20 wt %, 30 wt %, 40 wt % or 50 wt % of the GSG composition.

In some embodiments, the GSG composition comprises one or more unreacted SGs in an individual or total amount that equals to, or is less than 0.01 wt %, 0.1 wt %, 1 wt %, 2 wt %, 5 wt %, 10 wt %, 20 wt %, 30 wt %, 40 wt % or 50 wt % of the GSG composition.

In some embodiments, the GSG composition comprises unreacted dextrins and/or maltodextrin in an amount that equals to, or is less than 0.01 wt %, 0.1 wt %, 1 wt %, 2 wt %, 5 wt %, 10 wt %, 20 wt %, 30 wt %, 40 wt % or 50 wt % of the GSG composition.

In some embodiments, the GSG composition is a GRA, GRB, GRC, GRD, GRE, GRF, GRM, GRN, GRU, GDA, GSTV, GSTB or GSTM composition.

The glycosylation product of the present application may comprise both reacted and unreacted components from the starting materials (i.e., the mixture of materials before the initiation of the glycosylation reaction). In some embodiments, the glycosylation reaction product of the present application comprises GRA, GRB, GRC, GRD, GRE, GRF, GRM, GRN, GRU, GDA, GSTV, GSTB or GSTM in a range between 1-99.5 wt %, 1-5 wt %, 1-10 wt %, 1-20 wt %, 1-30 wt %, 1-40 wt %, 1-50 wt %, 1-60 wt %, 1-70 wt %, 1-80 wt %, 1-90 wt %, 1-99 wt %, 5-10 wt %, 5-20 wt %, 5-30 wt %, 5-40 wt %, 5-50 wt %, 5-60 wt %, 5-70 wt %, 5-80 wt %, 5-90 wt %, 5-99 wt %, 10-20 wt %, 10-30 wt %, 10-40 wt %, 10-50 wt %, 10-60 wt %, 10-70 wt %, 10-80 wt %, 10-90 wt %, 10-99 wt %, 20-30 wt %, 20-40 wt %, 20-50 wt %, 20-60 wt %, 20-70 wt %, 20-80 wt %, 20-90 wt %, 20-99 wt %, 30-40 wt %, 30-50 wt %, 30-60 wt %, 30-70 wt %, 30-80 wt %, 30-90 wt %, 30-99 wt %, 40-50 wt %, 40-60 wt %, 40-70 wt %, 40-80 wt %, 40-90 wt %, 40-99 wt %, 50-60 wt %, 50-70 wt %, 50-80 wt %, 50-90 wt %, 50-99 wt %, 60-70 wt %, 60-80 wt %, 60-90 wt %, 60-99 wt %, 70-80 wt %, 70-90 wt %, 70-99 wt %, 80-90 wt %, 80-99 wt %, 90-99 wt % of the of the glycosylation reaction product.

In some embodiments, GRA, GRB, GRC, GRD, GRE, GRF, GRM, GRN, GRU, GDA, GSTV, GSTB or GSTM is present in the glycosylation reaction product in an amount that equals to, or is greater than, 0.01 wt %, 0.1 wt %, 1 wt %, 2 wt %, 5 wt %, 10 wt %, 20 wt %, 30 wt %, 40 wt %, 50 wt %, 60 wt %, 70 wt %, 80 wt %, 90 wt %, 95 wt %, or 99 wt % of the glycosylation reaction product.

In some embodiments, unreacted RA, RB, RC, RD, RE, RF, RM, RN, RU, DA, STV, STB or STM is present in the glycosylation reaction product in an amount that equals to, or less than 0.01 wt %, 0.1 wt %, 1 wt %, 2 wt %, 5 wt %, 10 wt %, 20 wt %, 30 wt %, 40 wt %, 50 wt % or 60 wt % of the glycosylation reaction product.

In some embodiments, the glycosylation reaction product includes GSG and residual SGs at a GSG:residual SG weight ratio of 99:1 to 1:99, 90:1 to 1:99, 80:1 to 1:99, 70:1 to 1:99, 60:1 to 1:99, 50:1 to 1:99, 40:1 to 1:99, 30:1 to 1:99, 20:1 to 1:99, 10:1 to 1:99, 1:1 to 1:99, 99:1 to 1:90, 90:1 to 1:90, 80:1 to 1:90, 70:1 to 1:90, 60:1 to 1:90, 50:1 to 1:90, 40:1 to 1:90, 30:1 to 1:90, 20:1 to 1:90, 10:1 to 1:90, 1:1 to 1:90, 99:1 to 1:60, 90:1 to 1:60, 80:1 to 1:60, 70:1 to 1:60, 60:1 to 1:60, 50:1 to 1:60, 40:1 to 1:60, 30:1 to 1:60, 20:1 to 1:60, 10:1 to 1:60, 1:1 to 1:60, 99:1 to 1:30, 90:1 to 1:30, 80:1 to 1:30, 70:1 to 1:30, 60:1 to 1:30, 50:1 to 1:30, 40:1 to 1:30, 30:1 to 1:30, 20:1 to 1:30, 10:1 to 1:30, 1:1 to 1:30, 99:1 to 1:10, 90:1 to 1:10, 80:1 to 1:10, 70:1 to 1:10, 60:1 to 1:10, 50:1 to 1:10, 40:1 to 1:10, 30:1 to 1:10, 20:1 to 1:10, 10:1 to 1:10, 1:1 to 1:10.

The glycosylation product of the present application may comprise both reacted and unreacted components from the starting materials (i.e., the mixture of materials before the initiation of the glycosylation reaction). In some embodiments, the glycosylation reaction product of the present application comprises GSG in a range between 1-99.5 wt %, 1-5 wt %, 1-10 wt %, 1-20 wt %, 1-30 wt %, 1-40 wt %, 1-50 wt %, 1-60 wt %, 1-70 wt %, 1-80 wt %, 1-90 wt %, 1-99 wt %, 5-10 wt %, 5-20 wt %, 5-30 wt %, 5-40 wt %, 5-50 wt %, 5-60 wt %, 5-70 wt %, 5-80 wt %, 5-90 wt %, 5-99 wt %, 10-20 wt %, 10-30 wt %, 10-40 wt %, 10-50 wt %, 10-60 wt %, 10-70 wt %, 10-80 wt %, 10-90 wt %, 10-99 wt %, 20-30 wt %, 20-40 wt %, 20-50 wt %, 20-60 wt %, 20-70 wt %, 20-80 wt %, 20-90 wt %, 20-99 wt %, 30-40 wt %, 30-50 wt %, 30-60 wt %, 30-70 wt %, 30-80 wt %, 30-90 wt %, 30-99 wt %, 40-50 wt %, 40-60 wt %, 40-70 wt %, 40-80 wt %, 40-90 wt %, 40-99 wt %, 50-60 wt %, 50-70 wt %, 50-80 wt %, 50-90 wt %, 50-99 wt %, 60-70 wt %, 60-80 wt %, 60-90 wt %, 60-99 wt %, 70-80 wt %, 70-90 wt %, 70-99 wt %, 80-90 wt %, 80-99 wt %, 90-99 wt % of the of the total GRA.

In some embodiments, GSG is present in the glycosylation reaction product in an amount that equals to, or is greater than, 0.01 wt %, 0.1 wt %, 1 wt %, 2 wt %, 5 wt %, 10 wt %, 20 wt %, 30 wt %, 40 wt %, 50 wt %, 60 wt %, 70 wt %, 80 wt %, 90 wt %, 95 wt %, or 99 wt % of the glycosylation reaction product.

In some embodiments, unreacted SG is present in the glycosylation reaction product in an amount that equals to, or less than 0.01 wt %, 0.1 wt %, 1 wt %, 2 wt %, 5 wt %, 10 wt %, 20 wt %, 30 wt %, 40 wt %, 50 wt % or 60 wt % of the glycosylation reaction product.

In some embodiments, unreacted dextrin and/or maltodextrin is present in the glycosylation reaction product in an amount that equals to, or less than 0.01 wt %, 0.1 wt %, 1 wt %, 2 wt %, 5 wt %, 10 wt %, 20 wt % or 30 wt % of the glycosylation reaction product.

The GSG molecules of the present application include GSG molecules with different levels of glycosylation. In some embodiments, the GSG molecules of the present application comprise 1-20 additional monosaccharide units that are added to the SG backbone during a man-made glycosylation reaction. In some embodiments, the additional monosaccharide units are glucose units. In some embodiments, the additional monosaccharide units are non-glucose units, such as fructose, xylose and galactose units. In some embodiments, the additional monosaccharide units are a mixture of glucose units and non-glucose units. In some embodiments, the GSG of the present application comprises mono-glycosylated SG, di-glycosylated SG, tri-glycosylated SG, tetra-glycosylated SG and/or penta-glycosylated SG.

In some embodiments, the GSG composition of the present application contains mono-glycosylated SG, di-glycosylated SG, tri-glycosylated SG, tetra-glycosylated SG and/or penta-glycosylated SG, individually or in combination, in an amount of less than 99 wt %, 90 wt %, 80 wt %, 70 wt %, 65 wt %, 60 wt %, 55 wt %, 50 wt %, 45 wt %, 40 wt %, 35 wt %, 30 wt %, 25 wt %, 20 wt %, 15 wt %, 10 wt %, 5 wt %, 2 wt %, or 1 wt % of mono-glycosylated SG.

In some embodiments, the GSG composition of the present application contains mono-glycosylated SG, di-glycosylated SG, tri-glycosylated SG, tetra-glycosylated SG and/or penta-glycosylated SG, individually or in combination, in an amount that equals to, or is greater than, 1 wt %, 2 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt % or 80 wt % of the total GSG.

In some embodiments, the GSG composition of the present application contains mono-glycosylated SG, di-glycosylated SG, tri-glycosylated SG, tetra-glycosylated SG and/or penta-glycosylated SG, individually or in combination, in an amount that is in the range of 1-99 wt %, 1-95 wt %, 1-90 wt %, 1-85 wt %, 1-80 wt %, 1-75 wt %, 1-70 wt %, 1-65 wt %, 1-60 wt %, 1-55 wt %, 1-50 wt %, 1-45 wt %, 1-40 wt %, 1-35 wt %, 1-30 wt %, 1-25 wt %, 1-20 wt %, 1-15 wt %, 1-10 wt %, 1-5 wt %, 1-2 wt %, 2-99 wt %, 2-95 wt %, 2-90 wt %, 2-85 wt %, 2-80 wt %, 2-75 wt %, 2-70 wt %, 2-65 wt %, 2-60 wt %, 2-55 wt %, 2-50 wt %, 2-45 wt %, 2-40 wt %, 2-35 wt %, 2-30 wt %, 2-25 wt %, 2-20 wt %, 2-15 wt %, 2-10 wt %, 2-5 wt %, 5-99 wt %, 5-95 wt %, 5-90 wt %, 5-85 wt %, 5-80 wt %, 5-75 wt %, 5-70 wt %, 5-65 wt %, 5-60 wt %, 5-55 wt %, 5-50 wt %, 5-45 wt %, 5-40 wt %, 5-35 wt %, 5-30 wt %, 5-25 wt %, 5-20 wt %, 5-15 wt %, 5-10 wt %, 10-99 wt %, 10-95 wt %, 10-90 wt %, 10-85 wt %, 10-80 wt %, 10-75 wt %, 10-70 wt %, 10-65 wt %, 10-60 wt %, 10-55 wt %, 10-50 wt %, 10-45 wt %, 10-40 wt %, 10-35 wt %, 10-30 wt %, 10-25 wt %, 10-20 wt %, 10-15 wt %, 15-99 wt %, 15-95 wt %, 15-90 wt %, 15-85 wt %, 15-80 wt %, 15-75 wt %, 15-70 wt %, 15-65 wt %, 15-60 wt %, 15-55 wt %, 15-50 wt %, 15-45 wt %, 15-40 wt %, 15-35 wt %, 15-30 wt %, 15-25 wt %, 15-20 wt %, 20-99 wt %, 20-95 wt %, 20-90 wt %, 20-85 wt %, 20-80 wt %, 20-75 wt %, 20-70 wt %, 20-65 wt %, 20-60 wt %, 20-55 wt %, 20-50 wt %, 20-45 wt %, 20-40 wt %, 20-35 wt %, 20-30 wt %, 20-25 wt %, 25-99 wt %, 25-95 wt %, 25-90 wt %, 25-85 wt %, 25-80 wt %, 25-75 wt %, 25-70 wt %, 25-65 wt %, 25-60 wt %, 25-55 wt %, 25-50 wt %, 25-45 wt %, 25-40 wt %, 25-35 wt %, 25-30 wt %, 30-99 wt %, 30-95 wt %, 30-90 wt %, 30-85 wt %, 30-80 wt %, 30-75 wt %, 30-70 wt %, 30-65 wt %, 30-60 wt %, 30-55 wt %, 30-50 wt %, 30-45 wt %, 30-40 wt %, 30-35 wt %, 35-99 wt %, 35-95 wt %, 35-90 wt %, 35-85 wt %, 35-80 wt %, 35-75 wt %, 35-70 wt %, 35-65 wt %, 35-60 wt %, 35-55 wt %, 35-50 wt %, 35-45 wt %, 35-40 wt %, 40-99 wt %, 40-95 wt %, 40-90 wt %, 40-85 wt %, 40-80 wt %, 40-75 wt %, 40-70 wt %, 40-65 wt %, 40-60 wt %, 40-55 wt %, 40-50 wt %, 40-45 wt %, 45-99 wt %, 45-95 wt %, 45-90 wt %, 45-85 wt %, 45-80 wt %, 45-75 wt %, 45-70 wt %, 45-65 wt %, 45-60 wt %, 45-55 wt %, 45-50 wt %, 50-99 wt %, 50-95 wt %, 50-90 wt %, 50-85 wt %, 50-80 wt %, 50-75 wt %, 50-70 wt %, 50-65 wt %, 50-60 wt %, 50-55 wt %, 55-99 wt %, 55-95 wt %, 55-90 wt %, 55-85 wt %, 55-80 wt %, 55-75 wt %, 55-70 wt %, 55-65 wt %, 55-60 wt %, 60-99 wt %, 60-95 wt %, 60-90 wt %, 60-85 wt %, 60-80 wt %, 60-75 wt %, 60-70 wt %, 60-65 wt %, 65-99 wt %, 65-95 wt %, 65-90 wt %, 65-85 wt %, 65-80 wt %, 65-75 wt %, 65-70 wt %, 70-99 wt %, 70-95 wt %, 70-90 wt %, 70-85 wt %, 70-80 wt %, 70-75 wt %, 75-99 wt %, 75-95 wt %, 75-90 wt %, 75-85 wt %, 75-80 wt %, 80-99 wt %, 80-95 wt %, 80-90 wt %, 80-85 wt %, 85-99 wt %, 85-95 wt %, 85-90 wt %, 90-99 wt %, 90-95 wt % or 95-99 wt % of the total GSG.

In some embodiments, the GSG composition contains less than 60 wt %, 50 wt %, 40 wt %, 30 wt %, 20 wt %, 15 wt %, 10 wt %, 5 wt % or 2 wt % of mono-glycosylated SG. In some embodiments, the GSG contains greater than 5 wt %, 10 wt %, 15 wt %, 20 wt %, 30 wt %, 40 wt %, 50 wt % or 60 wt % of mono-glycosylated SG. In some embodiments, the GSG contain about 5 wt %, 10 wt %, 15 wt %, 20 wt %, 30 wt %, 40 wt %, 50 wt % or 60 wt % of mono-glycosylated SG.

In some embodiments, the GSG composition contains less than 60 wt %, 50 wt %, 40 wt %, 30 wt %, 20 wt %, 15 wt %, 10 wt %, 5 wt % or 2 wt % of di-glycosylated SG. In some embodiments, the GSG contains greater than 5 wt %, 10 wt %, 15 wt %, 20 wt %, 30 wt %, 40 wt %, 50 wt % or 60 wt % of di-glycosylated SG. In some embodiments, the GSG contain about 5 wt %, 10 wt %, 15 wt %, 20 wt %, 30 wt %, 40 wt %, 50 wt % or 60 wt % of di-glycosylated SG.

In some embodiments, the GSG composition contains less than 60 wt %, 50 wt %, 40 wt %, 30 wt %, 20 wt %, 15 wt %, 10 wt %, 5 wt % or 2 wt % of tri-glycosylated SG. In some embodiments, the GSG contains greater than 5 wt %, 10 wt %, 15 wt %, 20 wt %, 30 wt %, 40 wt %, 50 wt % or 60 wt % of tri-glycosylated SG. In some embodiments, the GSG contain about 5 wt %, 10 wt %, 15 wt %, 20 wt %, 30 wt %, 40 wt %, 50 wt % or 60 wt % of tri-glycosylated SG.

In some embodiments, the GSG composition contains less than 60 wt %, 50 wt %, 40 wt %, 30 wt %, 20 wt %, 15 wt %, 10 wt %, 5 wt % or 2 wt % of tetra-glycosylated SG. In some embodiments, the GSG contains greater than 5 wt %, 10 wt %, 15 wt %, 20 wt %, 30 wt %, 40 wt %, 50 wt % or 60 wt % of tetra-glycosylated SG. In some embodiments, the GSG contains about 5 wt %, 10 wt %, 15 wt %, 20 wt %, 30 wt %, 40 wt %, 50 wt % or 60 wt % of tetra-glycosylated SG.In some embodiments, the GSG composition contains less than 60 wt %, 5

0 wt %, 40 wt %, 30 wt %, 20 wt %, 15 wt %, 10 wt %, 5 wt % or 2 wt % of penta-glycosylated SG. In some embodiments, the GSG contains greater than 5 wt %, 10 wt %, 15 wt %, 20 wt %, 30 wt %, 40 wt %, 50 wt % or 60 wt % of penta-glycosylated SG. In some embodiments, the GSG contain about 5 wt %, 10 wt %, 15 wt %, 20 wt %, 30 wt %, 40 wt %, 50 wt % or 60 wt % of penta-glycosylated SG.

In some embodiments, the GSG composition is a glycosylation product with glucose as sugar donor (glucosylation product). In some embodiments, the GSG composition is a glycosylation product with arabinose as sugar donor (arabinosylation product). In some embodiments, the GSG composition is a glycosylation product with fructose as sugar donor (fructosylation product). In some embodiments, the GSG composition is a glycosylation product with galactose as sugar donor (galactosylation product). In some embodiments, the GSG composition is a glycosylation product with lactose as sugar donor (lactosylation product). In some embodiments, the GSG composition is a glycosylation product with mannose as sugar donor (mannosylation product). In some embodiments, the GSG composition is a glycosylation product with rhamnose as sugar donor (rhamnosylation product). In some embodiments, the GSG composition is a glycosylation product with xylase as sugar donor (xylosylation product).

In some embodiments, the GSG of the present application comprise a mixture of two, three or more glycosylation products selected from the group consisting of glucosylation products, arabinosylation products, fructosylation products, galactosylation products, lactosylation products, mannosylation products, rhamnosylation products, and xylosylation products. In some embodiments, the GSG of the present application comprise two glycosylation products mixed at a weight ratio of in the range of 99:1 to 1:99, 90:1 to 1:99, 80:1 to 1:99, 70:1 to 1:99, 60:1 to 1:99, 50:1 to 1:99, 40:1 to 1:99, 30:1 to 1:99, 20:1 to 1:99, 10:1 to 1:99, 1:1 to 1:99, 99:1 to 1:90, 90:1 to 1:90, 80:1 to 1:90, 70:1 to 1:90, 60:1 to 1:90, 50:1 to 1:90, 40:1 to 1:90, 30:1 to 1:90, 20:1 to 1:90, 10:1 to 1:90, 1:1 to 1:90, 99:1 to 1:60, 90:1 to 1:60, 80:1 to 1:60, 70:1 to 1:60, 60:1 to 1:60, 50:1 to 1:60, 40:1 to 1:60, 30:1 to 1:60, 20:1 to 1:60, 10:1 to 1:60, 1:1 to 1:60, 99:1 to 1:30, 90:1 to 1:30, 80:1 to 1:30, 70:1 to 1:30, 60:1 to 1:30, 50:1 to 1:30, 40:1 to 1:30, 30:1 to 1:30, 20:1 to 1:30, 10:1 to 1:30, 1:1 to 1:30, 99:1 to 1:10, 90:1 to 1:10, 80:1 to 1:10, 70:1 to 1:10, 60:1 to 1:10, 50:1 to 1:10, 40:1 to 1:10, 30:1 to 1:10, 20:1 to 1:10, 10:1 to 1:10, 1:1 to 1:10. In some embodiments, the two glycosylation products are fructosylation product and glucosylation product.

### B. Maillard Reaction Product Prepared From the SGs and GSGs of The Present Application

### (I) The Maillard Reaction

The Maillard reaction generally refers to a non-enzymatic browning reaction of a sugar donor with an amine donor in the presence of heat which produces flavor. Common flavors produced as a result of the Maillard reaction include, for example, those associated with red meat, poultry, coffee, vegetables, bread crust etc. subjected to heat. A Maillard reaction relies mainly on sugars and amino acids but it can also contain other ingredients including: autolyzed yeast extracts, hydrolyzed vegetable proteins, gelatin (protein source), vegetable extracts (i.e., onion powder), enzyme treated proteins, meat fats or extracts and acids or bases to adjust the pH of the reaction. The reaction can be in an aqueous environment with an adjusted pH at specific temperatures for a specified amount of time to produce a variety of flavors. Typical flavors include those associated with chicken, pork, beef, caramel, chocolate etc. However, a wide variety of different taste and aroma profiles can be achieved by adjusting the ingredients, the temperature and/or the pH of the reaction. The main advantage of the reaction flavors is that they can produce characteristic meat, burnt, roasted, caramellic, or chocolate profiles desired by the food industry, which are not typically achievable by using compounding of flavor ingredients.

Reducing groups can be found on reducing sugars (sugar donors) and amino groups can be found on amino donors such as free amino acids, peptides, and proteins. Initially, a reactive carbonyl group of a reducing sugar condenses with a free amino group, with a concomitant loss of a water molecule. A reducing sugar substrate for Maillard reaction typically has a reactive carbonyl group in the form of a free aldehyde or a free ketone. The resultant N-substituted glycoaldosylamine is not stable. The aldosylamine compound rearranges, through an Amadori rearrangement, to form a ketosamine. Ketosamines that are so-formed may further react through any of the following three pathways: (a) further dehydration to form reductones and dehydroreductones; (b) hydrolytic fission to form short chain products, such as diacetyl, acetol, pyruvaldehyde, and the like, which can, in turn, undergo Strecker degradation with additional amino groups to form aldehydes, and condensation, to form aldols; and (c) loss of water molecules, followed by reaction with additional amino groups and water, followed by condensation and/or polymerization into melanoids. Factors that affect the rate and/or extent of Maillard reactions include among others the temperature, water activity, and pH. The Maillard reaction is enhanced by high temperature, low moisture levels, and alkaline pH.

In the Maillard reaction, suitable carbonyl containing reactants include those that comprise a reactive aldehyde (--CHO) or keto (--CO--) group, such that the carbonyl free aldehyde or free keto group is available to react with an amino group associated with the reactant. Typically, the reducing reactant is a reducing sugar, e.g., a sugar that can reduce a test reagent, e.g., can reduce Cu2+ to Cu+, or can be oxidized by such reagents.

Monosaccharides, disaccharides, oligosaccharides, polysaccharides (e.g., dextrins, starches, and edible gums) and their hydrolysis products are suitable reducing reactants if they have at least one reducing group that can participate in a Maillard reaction. Reducing sugars include aldoses or ketoses such as glucose, fructose, maltose, lactose, glyceraldehyde, dihydroxyacetone, arabinose, xylose, ribose, mannose, erythrose, threose, and galactose. Other reducing reactants include uronic acids (e.g., glucuronic acid, glucuronolactone, and galacturonic acid, mannuronic acid, iduronic acid) or Maillard reaction intermediates bearing at least one carbonyl group such as aldehydes, ketones, alpha-hydroxycarbonyl or dicarbonyl compounds.

In some embodiments, the present application provides a Maillard reaction product (MRP) composition that is formed from heating a reaction mixture comprising (1) a SG and/or GSG and (2) one or more amine donors. In some embodiments, the reaction mixture further comprises (3) one or more sugar donors.

### (A) Amine Donor of a Maillard Reaction

The amine donor can be any compound or substance that contains a free amino group and that can participate in a Maillard reaction. Amine containing reactants include amino acids, peptides (including dipeptides, tripeptides, and oligopeptides), proteins, proteolytic or nonenzymatic digests thereof, and other compounds that react with reducing sugars and similar compounds in a Maillard reaction, such as phospholipids, chitosan, lipids, etc. In some embodiments, the amine donor also provides one or more sulfur-containing groups. Exemplary amine donors include amino acids, peptides, proteins, protein extracts.

Exemplary amino acids include, for example, nonpolar amino acids, such as alanine, glycine, isoleucine, leucine, methionine, tryptophan, phenylalanine, proline, valine; polar amino acids, such as cysteine, serine, threonine, tyrosine, asparagine, and glutamine; polar basic (positively charged) amino acids, such as histidine and lysine; and polar acidic (negatively charged) amino acids, such as aspartate and glutamate.

Exemplary peptides include, for example, hydrolyzed vegetable proteins (HVPs) and mixtures thereof.

Exemplary proteins include, for example, sweet taste-modifying proteins, soy protein, sodium caseinate, whey protein, wheat gluten or mixtures thereof. Exemplary sweet taste-modifying proteins include, for example, thaumatin, monellin, brazzein, miraculin, curculin, pentadin, mabinlin, and mixtures thereof. In certain embodiments, the sweet-taste modifying proteins may be used interchangeably with the term “sweetener enhancer.”

Exemplary protein extracts include yeast extracts, plant extracts, bacterial extracts and the like.

The nature of the amino donor can play an important role in accounting for the many flavors produced from a Maillard reaction. In some embodiments, the amine donor may account for one or more flavors produced from a Maillard reaction. In some embodiments, a flavor may be produced from a Maillard reaction by using one or more amine donors, or a particular combination of an amine donor and sugar donor.

In certain embodiments, the amine donor is present in the compositions described herein in a range of from about 1 to about 99 weight percent, from about 1 to about 50 weight percent, from about 1 to about 10 weight percent, from about 2 to about 9 weight percent, from about 3 to about 8 weight percent, from about 4 to about 7 weight percent, from about 5 to about 6 weight percent and all values and ranges encompassed over the range of from about 1 to about 50 weight percent. In some embodiments, the amine donor is from a plant source, such as vegetable juice, fruit juice, berry juice, etc.

### (B) Sugar Donor of a Maillard Reaction

In some embodiments, the sugar donor is a reducing sugar. Reducing sugars for use in the present application include, for example, all monosaccharides and some disaccharides, which can be aldose reducing sugars or ketose reducing sugars. Typically, the reducing sugar may be selected from the group consisting of aldotetrose, aldopentose, aldohexose, ketotetrose, ketopentose, and ketohexose reducing sugars. Suitable examples of aldose reducing sugars include erythrose, threose, ribose, arabinose, xylose, lyxose, allose, altrose, glucose, mannose, gulose, idose, galactose and talose. Suitable examples of ketose reducing sugars include erythrulose, ribulose, xylulose, psicose, fructose, sorbose and tagatose. The aldose or the ketose may also be a deoxy-reducing sugar, for example, a 6-deoxy reducing sugar, such as fucose or rhamnose.

Specific monosaccharide aldoses include, for example, reducing agents include, for example, where at least one reducing sugar is a monosaccharide, or the one or more reducing sugars are selected from a group comprising monosaccharide reducing sugars, typically at least one monosaccharide reducing sugar is an aldose or a ketose.

Where the reducing sugar is a monosaccharide, the monosaccharide may be in the D- or L-configuration, or a mixture thereof. Typically, the monosaccharide is present in the configuration in which it most commonly occurs in nature. For example, the one or more reducing sugars may be selected from the group consisting of D-ribose, L-arabinose, D-xylose, D-lyxose, D-glucose, D-mannose, D-galactose, D-psicose, D-fructose, L-fucose and L-rhamnose. In a more particular embodiment, the one or more reducing sugars are selected from the group consisting of D-xylose, D-glucose, D-mannose, D-galactose, L-rhamnose and lactose.

Specific reducing sugars include ribose, glucose, fructose, maltose, lyxose, galactose, mannose, arabinose, xylose, rhamnose, rutinose, lactose, maltose, cellobiose, glucuronolactone, glucuronic acid, D-allose, D-psicose, xylitol, allulose, melezitose, D-tagatose, D-altrose, D-alditol, L-gulose, L-sorbose, D-talitol, inulin, stachyose, including mixtures and derivatives therefrom.

Exemplary disaccharide reducing sugars for use in the present application include maltose, lactose, lactulose, cellubiose, kojibiose, nigerose, sophorose, laminarbiose, gentiobiose, turanose, maltulose, palantinose, gentiobiulose, mannobiose, melibiose, melibiulose, rutinose, rutinulose or xylobiose.

Mannose and glucuronolactone or glucuronic acid can be used as sugar donors under Maillard reaction conditions, although they have seldom been used. Maillard reaction products of mannose, glucuronolactone or glucuronic acid provide yet another unique approach to provide new taste profiles with the sweetener agents described throughout the specification alone or in combination with additional natural sweeteners, synthetic sweeteners, and/or flavoring agents described herein.

In some embodiments, one or more carbohydrate sweeteners may be added to a reaction mixture subjected to the Maillard reaction. In other embodiments, one or more carbohydrate sweeteners may be added to an MRP composition after Maillard reaction. Non-limiting examples of carbohydrate sweeteners for use in the present application include caloric sweeteners, such as, sucrose, fructose, glucose, D-tagatose, trehalose, galactose, rhamnose, cyclodextrin (e.g., α-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, glucuronic acid, gluconic acid, glucono-lactone, abequose, galactosamine, sugar alcohols, such as erythritol, xylitol, mannitol, sorbitol, maltitol, lactitol, mannitol, and inositol; xylo-oligosaccharides (xylotriose, xylobiose and the like), gentio-oligoscaccharides (gentiobiose, gentiotriose, gentiotetraose and the like), galacto-oligosaccharides, sorbose, nigero-oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), lactulose, melibiose, raffinose, rhamnose, ribose, isomerized liquid sugars such as high fructose corn/starch syrup (containing fructose and glucose, e.g., HFCS55, HFCS42, or HFCS90), coupling sugars, soybean oligosaccharides, and glucose syrup. Additionally, the above carbohydrates may be in either the D- or L-configuration.

It should be noted, however, that not all carbohydrate sweeteners are reducing sugars. Sugars having acetal or ketal linkages are not reducing sugars, as they do not have free aldehyde chains. They therefore do not react with reducing-sugar test solutions (e.g., in a Tollens’ test or Benedict’s test). However, a non-reducing sugar can be hydrolyzed using diluted hydrochloric acid.

In some embodiments, the sugar donor is a non-reducing sugar that does not contain free aldehyde or free keto groups. Exemplary non-reducing sugars include, but are not limited to, sucrose, trehalose, xylitol, and raffinose. In some embodiments, the sugar donor comprises both reducing sugar and non-reducing sugar. In some embodiments, the sugar donor is derived from a food ingredient, such as sugar, flour, starch, vegetable and fruits.

In some embodiments, the sugar donor is derived from a plant source. For example, in some embodiments, the sugar donor comprises a fruit juice, berry juice, vegetable juice, syrup, plant extract, vegetable extract etc.

In some embodiments, the sugar donor is orange juice, cranberry juice, apple juice, peach juice, watermelon juice, pineapple juice, grape juice and concentrated products thereof.

In some embodiments, the fruit juice, berry juice or vegetable juice serves as both amine donor and sugar donor.

Reducing sugars can be derived from various sources for use as sugar donors in the Maillard reaction. For example, a sugar syrup may be extracted from a natural source, such as Monk fruit, fruit juice or juice concentrate (e.g., grape juice, apple juice, etc.), vegetable juice (e.g., onion etc.), or fruit (e.g., apples, pears, cherries, etc.) for use as a sugar donor.

The syrup may include any type of juice regardless of whether there is any ingredient being isolated from juice, such as purified apple juice with trace amounts of malic acid etc. The juice can be in the form of liquid, paste or solid. Sugar donors may also be extracted from Stevia, sweet tea, luohanguo, etc. after isolation of high intensity sweetener agents described herein (containing non-reducing sugars) from crude extracts and mixtures thereof. Extracts from any part of plant containing reducing sugars can be used as sugar donors in Maillard reactions with or without other additional reducing sugars. In some embodiments, the MRPs are prepared using a plant extract as a sugar donor.

In some embodiments, the sugar donor and amino donor are present in the reaction mixture in a molar ratio of 10:1 to 1:10, 8:1 to 1:8, 6:1 to 1:6, 4:1 to 1:4, 3:1 to 1:3 or 2:1 to 1:2. In some embodiments, the sugar donor and amino donor are present in the reaction mixture in a molar ratio of 10:1, 9:1, 8:1, 7:1, 6:1, 5:1, 4:1, 3:1, 2:1, 1:1, 1:2, 1:3, 1:4, 1:5, 1:6, 1:7, 1;8, 1:9 or 1:10.

In some embodiments, the sugar donor and amino donor are present in the reaction mixture in a sugar donor:amino donor weight ratio of 10:1 to 1:10, 8:1 to 1:8, 6:1 to 1:6, 4:1 to 1:4, 3:1 to 1:3 or 2:1 to 1:2. In some embodiments, the sugar donor and amino donor are present in the reaction mixture in a molar ratio of 10:1, 9:1, 8:1, 7:1, 6:1, 5:1, 4:1, 3:1, 2:1, 1:1, 1:2, 1:3, 1:4, 1:5, 1:6, 1:7, 1;8, 1:9 or 1:10.

In some embodiments, the sugar donor in the MRP reaction comprises one or more members selected from the group consisting of fructose, arabinose, maltose, high maltose syrup, dextrin, maltodextrin, fructose, high fructose syrup, glucose, and high glucose syrup.

In some embodiments, the sugar donor in the MRP reaction comprises a monosaccharide or a disaccharide. In some embodiments, the sugar donor in the MRP reaction comprises a fruit juice, a vegetable juice or honey.

### (C) Additional Components in the Reaction Mixture of Maillard Reaction

In some embodiments, the reactants for the Maillard reaction include a number of different raw materials for producing the MRP compositions of the present application. The raw materials may be categorized into the following groups comprising the following exemplary materials:

(1) Protein nitrogen sources:

Protein nitrogen containing foods (meat, poultry, eggs, dairy products, cereals, vegetable products, fruits, yeasts), extracts thereof and hydrolysis products thereof, autolyzed yeasts, peptides, amino acids and/or their salts.

(2) Carbohydrate sources:

Foods containing carbohydrates (cereals, vegetable products and fruits) and their extracts; mono-, di- and polysaccharides (sugars, dextrins, starches and edible gums), and hydrolysis products thereof.

(3) Fat or fatty acid sources:

Foods containing fats and oils, edible fats and oil from animal, marine or vegetable origin, hydrogenated, trans-esterified and/or fractionated fats and oils, and hydrolysis products thereof.

4) Miscellaneous list of additional ingredients:
Foodstuffs, herbs, spices, their extracts and flavoring agents identified therein
Water
Thiamine and its hydrochloric salt
Ascorbic, Citric, Lactic, Fumaric, Malic, Succinic, Tartaric and the Na, K, Ca, Mg and NH4 salts of these acids
Guanylic acid and inosinic acid and its Na, K and Ca salts
Inositol
Sodium, potassium and ammonium sulphides, hydrosulphides and polysulphides
Lecithin
Acids, bases and salts as pH regulators:
Acetic, hydrochloric, phosphoric and sulphuric acids
Sodium, potassium, calcium and ammonium hydroxide.
Salts of the above acids and bases
Polymethylsiloxane as antifoaming agent.

In another aspect, the present application contemplates the use of any one of a number of raw materials exemplified below to produce NATURAL PRODUCTS:

Sugar Syrups: Xylose syrup, arabinose syrup and rhamnose syrup manufactured from beech wood. Ardilla Technologies supply these along with natural crystalline L-xylose, L-arabinose and L-rhamnose. Xylose syrup may also be obtained from natural sources, such as the xylan-rich portion of hemicellulose, mannose syrup from ivory nut, etc. These and other types of syrup described herein can be used as sugar donors in the compositions described herein.

Hydrolyzed gum arabic: Thickeners, such as gum arabic can be hydrolyzed with an organic acid or by enzyme hydrolysis to produce a mixture containing arabinose. Arabinose could also be obtained from other wood-based or biomass hydrolysate. Cellulose enzymes can also be used.

Meat Extracts: Commercially available from a number of companies, such as Henningsens (Chicken skin and meat), which gives excellent chicken notes.

Jardox: Meat and poultry extracts and stocks.

Kanegrade: Fish powders, anchovy, squid, tuna and others.

Vegetable Powders: onion and garlic powders, celery, tomato and leek powders are effective flavor contributors to reaction flavors.

Egg Yolk: Contains 50% fat and 50% protein. The fat contains phospholipids and lecithin. The proteins are coagulating proteins and their activity must be destroyed by hydrolysis with acid or by the use of proteases prior to use. This will also liberate amino acids and peptides useful in reaction flavors (Allergen activity).

Vegetable oils: Peanut (groundnut) oil - Oleic acid 50%, Linoleic acid 32% -beef and lamb profile. Sunflower - linoleic acid 50 - 75%, oleic 25% - chicken profile. Canola (rapeseed) - oleic 60%, linoleic 20%, alpha-linoleic 10%, gadoleic 12%.

Sauces: Fish sauce, soy sauce, oyster sauce, miso.

Enzyme Digests: Beef heart digest - rich in phospholipids. Liver digest - at low levels <5% gives a rich meaty character. Meat digests can also add authenticity but they are usually not as powerful as yeast extracts and HVPs.

Enzyme enhanced umami products - shitake or porcini mushrooms, kombu, etc. Enzyme digested fats - beef, lamb, etc.

All of the components of the compositions disclosed herein can be purchased or made by processes known to those of ordinary skill in the art and combined (e.g., precipitation/co-precipitation, mixing, blending, grounding, mortar and pestle, microemulsion, solvothermal, sonochemical, etc.) or treated as defined by the current invention.

### (D) Solvent

The Maillard reaction is conducted with a suitable solvent or carrier. Examples of suitable solvents or carriers include but are not limited to water, alcohols such as low molecular weight alcohols (e.g., methanol, ethanol, propanol, butanol, pentanol, hexanol, ethylene glycol, propylene glycol, butyl glycol, etc.), acetone, benzyl alcohol, 1,3-butylene glycol, carbon dioxide, castor oil, citric acid esters of mono- and di-glycerides, ethyl acetate, ethyl alcohol, ethyl alcohol denatured with methanol, glycerol (glycerin), glyceryl diacetate, glyceryl triacetate (triacetin), glyceryl tributyrate (tributyrin), hexane, isopropyl alcohol, methyl alcohol, methyl ethyl ketone (2-butanone), methylene chloride, monoglycerides and diglycerides, monoglyceride citrate, 1,2-propylene glycol, propylene glycol mono-esters and diesters, triethyl citrate, and mixtures thereof.

Although recognizing that other suitable solvents may be used for flavoring agents, The International Organization of the Flavor Industry (IOFI) Code of Practice (Version 1.3, dated Feb. 29, 2012) lists the following solvents as being appropriate for use in flavoring agents: acetic acid, benzyl alcohol, edible oils, ethyl alcohol, glycerol, hydrogenated vegetable oils, isopropyl alcohol, mannitol, propylene glycol, sorbitol, sorbitol syrup, water, and xylitol. Accordingly, in certain embodiments, these are preferred solvents.

In some embodiments, the solvent is water. In some embodiments, the solvent is glycerol. In some embodiments, the solvent is a glycerol-water mixture with a glycerol:water ratio (v:v) of 10:1 to 1:10, 9:1 to 1:9, 8:1 to 1:8, 7:1 to 1:7, 6:1 to 1:6, 1:5 to 5:1, 1:4 to 4:1, 1:3 to 3:1, 1:2 to 2:1. In some embodiments, the solvent is a glycerol-water mixture with a glycerol:water ratio (v:v) of 1:9, 1:8, 1:7, 1:6, 1:5, 1:4, 1:3, 1:2, 1:1, 2:1, 3:1, 4:1, 5:1, 6:1, 7:1, 8:1 or 9:1.

In some embodiments, the reaction mixture comprises a solvent in an amount of 10-90 wt %, 10-80 wt %, 10-70 wt %, 10-60 wt %, 10-50 wt %, 10-40 wt %, 10-30 wt %, 10-20 wt %, 20-90 wt %, 20-80 wt %, 20-70 wt %, 20-60 wt %, 20-50 wt %, 20-40 wt %, 20-30 wt %, 30-90 wt %, 30-80 wt %, 30-70 wt %, 30-60 wt %, 30-50 wt %, 30-40 wt %, 40-90 wt %, 40-80 wt %, 40-70 wt %, 40-60 wt %, 40-50 wt %, 50-90 wt %, 50-80 wt %, 50-70 wt %, 50-60 wt %, 60-90 wt %, 60-80 wt %, 60-70 wt %, 70-90 wt %, 70-80 wt %, or 80-90 wt % of the reaction mixture. In some embodiments, the reaction mixture comprises a solvent in an amount of about 10 wt %, about 15 wt %, about 20 wt %, about 25 wt %, about 30 wt %, about 33 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt % of the reaction mixture.

### (II) Maillard Reaction Conditions

Maillard reaction conditions are affected by temperature, pressure, pH, reaction times, ratio of different reactants, types of solvents, and solvents-to-reactants ratio. Accordingly, in certain embodiments, the reaction mixture may include a pH regulator, which can be an acid or a base. Suitable base regulators include, for example, sodium hydroxide, potassium hydroxide, baking powder, baking soda, any useable food grade base salts including alkaline amino acids. Additionally, the Maillard reaction can be conducted in the presence of alkalinic amino acids without the need of an additional base where the alkaline amino acid serves as the base itself. The pH of the reaction mixture can be maintained at any pH suitable for the Maillard reaction. In certain embodiments, the pH is maintained at a pH of from about 2 to about 14, from about 2 to about 7, from about 3 to about 9, from about 4 to about 8, from about 5 to about 7, from about 7 to about 14, from about 8 to about 10, from about 9 to about 11, from about 10 to about 12, or any pH range derived from these integer values.

In some embodiments, the reaction mixture has a pH of 4, 5, 6, 7, 8 or 9 at the initiation of the Maillard reaction.

In any of the embodiments described in the present application, the reaction temperature in any of the MRP reaction mixtures described in the present application may be 0° C., 5° C., 10° C., 20° C., 25° C., 30° C., 35° C., 40° C., 50° C., 55° C., 60° C., 65° C., 70° C., 80° C., 90° C., 100° C., 110° C., 120° C., 125° C., 130° C., 135° C., 140° C., 150° C., 155° C., 160° C., 165° C., 170° C., 180° C., 190° C., 200° C., 210° C., 220° C., 225° C., 230° C., 235° C., 240° C., 250° C., 255° C., 260° C., 265° C., 270° C., 280° C., 290° C., 300° C., 400° C., 500° C., 600° C., 700° C., 800° C., 900° C., 1000° C., or any temperature range defined by any two temperature values in this paragraph.

In more particular embodiments, the reaction temperature in any of the MRP reaction mixtures described in the present application may range from 0° C. to 1000° C., 10° C. to 300° C., from 15° C. to 250° C., from 20° C. to 250° C., from 40° C. to 250° C., from 60° C. to 250° C., from 80° C. to 250° C., from 100° C. to 250° C., from 120° C. to 250° C., from 140° C. to 250° C., from 160° C. to 250° C., from 180° C. to 250° C., from 200° C. to 250° C., from 220° C. to 250° C., from 240° C. to 250° C., from 30° C. to 225° C., from 50° C. to 225° C., from 70° C. to 225° C., from 90° C. to 225° C., from 110° C. to 225° C., from 130° C. to 225° C., from 150° C. to 225° C., from 170° C. to 225° C., from 190° C. to 225° C., from 210° C. to 225° C., from 80° C. to 200° C., from 100° C. to 200° C., from 120° C. to 200° C., from 140° C. to 200° C., from 140° C. to 200° C., from 160° C. to 200° C., from 180° C. to 200° C., from 90° C. to 180° C., from 100° C. to 180° C., from 110° C. to 180° C., from 120° C. to 180° C., from 130° C. to 180° C., from 140° C. to 180° C., from 150° C. to 180° C., from 160° C. to 180° C., from 80° C. to 160° C., from 90° C. to 160° C., from 100° C. to 160° C., from 110° C. to 160° C., from 120° C. to 160° C., from 130° C. to 160° C., from 140° C. to 160° C., from 150° C. to 160° C., from 80° C. to 140° C., from 90° C. to 140° C., from 100° C. to 140° C., from 110° C. to 140° C., from 120° C. to 140° C., from 130° C. to 140° C., from 80° C. to 120° C., from 85° C. to 120° C., from 90° C. to 120° C., from 95° C. to 120° C., from 100° C. to 120° C., from 110° C. to 120° C., from 115° C. to 120° C., from 80° C. to 100° C., from 85° C. to 100° C., from 90° C. to 100° C., from 95° C. to 100° C.; or any aforementioned temperature value in this paragraph, or a temperature range defined by any pair of the aforementioned temperature values in this paragraph.

Maillard reaction(s) can be conducted either under open or sealed conditions. The reaction time is generally from 1 second to 100 hours, more particularly from 1 minute to 24 hours, from 1 minute to 12 hours, from 1 minute to 8 hours, from 1 minute to 4 hours, from 1 minute to 2 hours, from 1 minute to 1 hour, from 1 minute to 40 minutes, from 1 minute to 20 minutes, from 1 minute to 10 minutes, from 10 minutes to 24 hours, from 10 minutes to 12 hours, from 10 minutes to 8 hours, from 10 minutes to 4 hours, from 10 minutes to 2 hours, from 10 minutes to 1 hour, from 10 minutes to 40 minutes, from 10 minutes to 20 minutes, from 20 minutes to 24 hours, from 20 minutes to 12 hours, from 20 minutes to 8 hours, from 20 minutes to 4 hours, from 20 minutes to 2 hours, from 20 minutes to 1 hour, from 20 minutes to 40 minutes, from 40 minutes to 24 hours, from 40 minutes to 12 hours, from 40 minutes to 8 hours, from 40 minutes to 4 hours, from 40 minutes to 2 hours, from 40 minutes to 1 hour, from 1 hour to 24 hours, from 1 hour to 12 hours, from 1 hour to 8 hours, from 1 hour to 4 hours, from 1 hour to 2 hours, from 2 hour to 24 hours, from 2 hour to 12 hours, from 2 hour to 8 hours, from 2 hour to 4 hours, from 4 hour to 24 hours, from 4 hour to 12 hours, from 4 hour to 8 hours, from 8 hour to 24 hours, from 8 hour to 12 hours, or from 12 hour to 24 hours. Depending on the desired taste, the reaction can be terminated at any time. The Maillard reaction mixture can contain unreacted reactants, degraded substances from the reactants, pH regulator(s), and/or salt(s).

The Maillard reactions can be conducted at atmospheric pressure or under pressure. When conducted under pressure, the reaction mixture may be subjected to constant pressure or it may be subjected to varying pressures over time. In certain embodiments, the pressure in the reaction vessel is at least 10 MPa, at least 20 MPa, at least 30 MPa, at least 40 MPa, at least 50 MPa, at least 75 MPa, at least 100 MPa, at least 150 MPa, at least 200 MPa, at least 250 MPa, at least 300 MPa, at least 400 MPa, at least 500 MPa, at least 600 MPa, at least 700 MPa, at least 800 MPa, and any pressure range derived from the aforementioned pressure values.

In some embodiments, the Maillard reaction is conducted with the assistance of microwave heating. Microwave heating results in the superheating of substances, particularly those that response to dipole rotation or ionic conductivity.

In some embodiments, it is desirable to suppress the Maillard reaction, in part. This can be achieved by exercising one or more of the following approaches, including the use of raw materials that are not susceptible to browning, adjusting the factors affecting the browning velocity of Maillard reaction, lowering the temperature, lowering pH, adjusting water activity, increasing the level of oxygen, using oxidant, introducing enzymes, etc.

In certain embodiments, the use of low solubility or insoluble amino acids in the Maillard reaction may result in insoluble reactants present in the final MRP composition. In such cases, filtration may be used to remove any insoluble components present in the MRP compositions.

A general method to prepare derived Maillard reaction product(s) is described as follows. Briefly, a steviol glycoside composition of the present application, such as RA or hpRA, or a glycosylated steviol glycoside composition of the present application, such as GRA or G-hpRA, is dissolved with or without a sugar donor, and together with amino acid donor in a solvent, such as water, to form a reaction mixture, followed by heating of the reaction mixture at an elevated temperature, for example, from 30, 40, or 50° C. up to 250° C. The reaction time can be varied from more than one minute to a few days, more generally a few hours, until Maillard reaction products (MRPs) are formed or one of the reaction components has been exhausted or the reaction has been completed, with or without formation of caramelization reaction products (CRPs), which are further described below. When required, a pH adjuster or pH buffer can be added to regulate the pH of the reaction mixture before, during or after reaction as further described herein. The resultant solution is dried by spray dryer or hot air oven to remove the water and to obtain the MRP composition of the present application.

When the reaction is completed, the product mixture does not need to be neutralized or it can be neutralized. Water and/or solvent(s) do not necessarily need to be removed but can be removed by distillation, spray drying or other known methods if the product is desired as a powder or liquid, whatever the case may be.

Interestingly, when a reaction mixture is dried to a powder, such as by spray drying, the resultant powder typically only has a slight smell associated with them. This is in contrast to regular powdered flavoring agents that generally have a strong smell. The dried powdered reaction mixtures of the embodiments, when dissolved in a solvent, such as water or alcohol or mixtures thereof, release the smell. This demonstrates that the volatile substances in the MRP can be preserved by the SG or GSG present in the MRP composition of the present application. Powders with strong aromas can be obtained too, particularly where the carrier, such as RA or GRA, is much less compared with MRP flavors or strong flavor substances used during Maillard reactions.

In some embodiments, the MRP composition may further include one or more carriers (or flavor carriers) acceptable for use with sweetener agents or flavoring agents. In addition, such carriers may be suitable e.g., as solvents for the Maillard reaction.

Exemplary carriers include acetylated distarch adipate, acetylated distarch phosphate, agar, alginic acid, beeswax, beta-cyclodextrine, calcium carbonate, calcium silicate, calcium sulphate, candelilla wax, carboxymethyl cellulose, sodium salt, carnauba wax, carrageenan, microcrystalline cellulose, dextran, dextrin, diammonium phosphate, distarch phosphate, edible fats, elemi resin, ethyl lactate, ethyl cellulose, ethyl hydroxyethyl cellulose, ethyl tartrate, gelatin, gellan gum, ghatti gum, glucose, glyceryl diacetate, glyceryl diesters of aliphatic fatty acids C6-C18, glyceryl monoesters of aliphatic fatty acids C6-C18, gyceryl triacetate (triacetin), glyceryl triesters of aliphatic fatty acids C6-C18, glyceryl tripropanoate, guar gum, gum arabic, hydrolyzed vegetable protein, hydroxyproplymethyl cellulose, hydroxypropyl cellulose, hydroxypropyl distarch phosphate, hydroxypropyl starch, karaya gum, konjac gum, lactic acid, lactose, locust bean gum (carob bean gum), magnesium carbonate, magnesium salts of fatty acids, maltodextrin, methyl cellulose, medium chain triglyceride, modified starches, such as acetylated distarch adipate, acetylated oxidized starch, acid-treated starch, alkaline treated starch, bleached starch, roasted starch dextrins, distarch phosphate, hydroxypropyl distarch phosphate, acetylated distarch phosphate, hydroxypropyl starch, monostarch phosphate, oxidized starch, phosphated distarch phosphate, starch acetate, starch sodium octenyl succinate, and enzyme treated starches; mono-,di- and tri-calcium orthophosphate, Na, K, NH4 and Ca alginate, pectins, processed euchema seaweed, propylene glycol alginate, sodium chloride (salt), silicon dioxide, sodium aluminium diphosphate, sodium aluminium silicate, Sodium, potassium and calcium salts of fatty acids, starch, starch (sodium) octenyl succinate, starch acetate, sucro glycerides, sucrose, sucrose esters of fatty acids, type I and type II sucrose oligoesters, taragum, tragacanth, triethylcitrate, whey powder, xanthan gum, fibers such as non-starch polysaccharides, lignin, cellulose, methylcellulose, the hemicelluloses, β-glucans, mucilage, inulins, oligosaccharides, polydextrose, fructooligosaccharides, cyclodextrins, chitins, and combinations thereof, and thickeners such as carbomers, cellulose base materials, gums, waxes, algin, agar, pectins, carrageenan, gelatin, mineral or modified mineral thickeners, polyethylene glycol and polyalcohols, polyacrylamide and other polymeric thickeners, and combinations thereof.

When utilizing the MRP compositions for use in a sweetener or flavoring composition, one or more additional components may be added to the MRP composition after the Maillard reaction has occurred. In some embodiments, these additional components include flavoring substances. Moreover, the reaction products after the Maillard reaction has been completed can further include, for example, one or more sweetener agents, reducing sugars (i.e., residue sugar donors), amine donors, sweetener enhancers, and CRPs, as well as one or more degraded sweetener agents, degraded sugar donors, degraded amine donors, and salts.

It should also be understood, for example, that the Maillard reaction can be performed under conditions containing an excess of amine donors in comparison to reducing sugars or much less than the amount of reducing sugars present. In the first instance, the resultant MRPs would include unreacted amine donors, degraded amine donors and/or residues from reacted amine donors. Conversely, when there is an excess of reducing sugars present in the Maillard reaction, the amine donors would be more fully reacted during the course of the reaction and a greater amount of unreacted reducing sugars as well as degraded reducing sugars and/or degrading reducing sugars and residues therefrom. Surprisingly, where the reducing sugar is replaced with a sweetener agent (e.g., a material such as an RA that does not include a reactive aldehydic or ketone moiety) and reacted with one or more amine donors, the amine donors may be present in the reaction products in reduced amounts reflecting their consumption in the Maillard type reaction or there excess of amine donors, as well as amine donor residues and/or amine degradation products after the Maillard reaction has been completed.

There are many ways to control the resulting MRP composition. For instance, adjusting the pH, pressure, reaction time, and ingredient additions to optimize the ratio of raw materials etc. Further, the separation of Maillard reaction products can provide a means for preparing different types of flavors or flavor enhancers. For example, a Maillard reaction product composition includes both volatile substances and non-volatile substances. Therefore, by evaporating the volatile substances, non-volatile substances can be purified for use. These non-volatile substances (or products) can be used as flavor modifiers or with the top note flavor in final products, such as volatile peach, lemon flavor provided by traditional flavor houses.

Volatile substances can be used as flavor or flavor enhancers as well. Partial separation/purification of a MRP can be carried out to obtain volatile substances, which can be further separated by distillation etc. or obtain non-volatile substances for instance by recrystallization, chromatograph etc. could be done to meet different targets of taste and flavor. Therefore, the MRP compositions of the present application include compositions containing one or more volatile substances, one or more non-volatile substances or mixtures thereof. Non-volatile substances in MRPs or isolated from MRPs can provide a good mouth feel, umami and Kokumi taste.

In some embodiments, the sweetener or flavoring composition of the present application further comprises a MRP formed from a reaction mixture comprising one or more flavonoid glycosides, isoflavone glycosides, saponin glycosides, phenol glycosides, cynophore glycosides, anthraquinone glycosides, cardiac glycosides, bitter glycosides, coumarin glycosides, and/or sulfur glycosides.

In some embodiments, the sweetener or flavoring composition further comprises a MRP formed from a reaction mixture comprising one or more glycosylated flavonoid glycosides, glycosylated isoflavone glycosides, glycosylated saponin glycosides, glycosylated phenol glycosides, glycosylated cynophore glycosides, glycosylated anthraquinone glycosides, glycosylated cardiac glycosides, glycosylated bitter glycosides, glycosylated coumarin glycosides, and/or glycosylated sulfur glycosides.

In some embodiments, the sugar donor may account for one or more flavors produced from a Maillard reaction. More particularly, a flavor may be produced from a Maillard reaction by using one or more sugar donors, wherein at least one sugar donor is selected from a product comprising a glycoside and a free carbonyl group. In some embodiments, glycosidic materials for use in Maillard reactions include natural juice/concentrates/extracts selected from strawberry, blueberry, blackberry, bilberry, raspberry, lingonberry, cranberry, red currants, white currants, blackcurrants, apple, peach, pear, apricot, mango, grape, water melon, cantolope, grapefruit, passion fruit, dragon fruit, carrot, celery, eggplant, tomato, etc.

The natural extracts used in Maillard reactions described herein can include any solvent extract-containing substances, such as polyphenols, free amino acids, flavonoids etc. The extracts can be further purified by methods such as resin-enriched, membrane filtration, crystallization etc., as further described herein.

In one embodiment, a Maillard reaction mixture or an MRP composition produced thereof may include a sweetener, a sweetener enhancer, such as thaumatin, and optionally one or more MRP products, wherein the sweetener is selected from date paste, apple juice concentrate, monk fruit concentrate, sugar beet syrup, pear juice or puree concentrate, apricot juice concentrate. Alternatively, a root or berry juice may be used as sugar donor or sweetener added to an MRP composition.

In some embodiments, particular flavors may be produced from a Maillard reaction through the use of one or more sugar donors, where at least one sugar donor is selected from plant juice/powder, vegetable juice/powder, berries juice/powder, fruit juice/powder. In certain preferred embodiments, a concentrate or extract may be used, such as a bilberry juice concentrate or extract having an abundance of anthocyanins. Optionally, at least one sugar donor and/or one amine donor is selected from animal source based products, such as meat, oil etc. Meat from any part of an animal, or protein(s) from any part of a plant could be used as source of amino donor(s) in this application.

In some embodiments, the Maillard reactants may further include one or more high intensity synthetic sweeteners, natural non-SG sweeteners, and/or the glycosylation products thereof. Alternatively, or in addition, the high intensity synthetic sweeteners may be added to an MRP composition comprising reaction products formed in the Maillard reaction.

Caramelization can occur in the course of Maillard reaction. Exemplary reactions include:
1. equilibration of anomeric and ring forms
2. sucrose inversion to fructose and glucose
3. condensation
4. intramolecular bonding
5. isomerization of aldoses to ketoses
6.dehydration reactions
7. fragmentation reactions
8. unsaturated polymer formation

One embodiment comprises one or more of these non-volatile substances originating from the MRP of the present application, including remaining sugar donors, remaining amine donors, and caramelized substances thereof. The caramelized substances can include e.g., caramelized disaccharides, trisaccharides, tetrasaccharides etc., which are formed by sugar donors; dimer-peptides, tri-peptides, tetra-peptides etc., which are formed by amine donors; glycosylamine and their derivatives, such as Amadori compounds, Heyns compounds, enolisated compounds, sugar fragments, amino acid fragments, and non-volatile flavor compounds formed by Maillard reactions of sugars and amino acid donors.

### (III) Precision Fermentation

In some embodiments, a desirable MRP or a glycosylated amine donor is prepared by precision fermentation. Precision fermentation technology is a form of synthetic biology that typically requires the use of genetically engineered microorganisms. The genetically engineered microorganisms, such as yeast, algae or bacteria, are capable of producing or excreting a particular desirable material, such as edible fats, proteins and glycosylated amines donors. In some embodiments, glycosylated conventional amine donors, glycosylated natural high intensity sweeteners, glycosylated stevia extracts, glycosylated SGs, glycosylated sweet tea extracts, or glycosylated monk fruit extracts, are produced by precision fermentation. In some embodiments, the C-MRPs, SG-MRPs or GSG-MRPs of the present application are produced by precision fermentation.

### (IV) MRP-Containing Compositions of the Present Application

In one embodiment, the composition of the present application includes (1) a Maillard reaction product (MRP) formed from a reaction mixture containing a NHIS and/or glycosylated NHIS, an amine donor, and optionally a sugar donor; and (2) a sweetener. The amine donor and/or sugar donor for use in the Maillard reaction may include any of the foregoing amine donors or sugar donors described above. In some embodiments, the sweetener comprises one or more components selected from the group consisting of NHIS, glycosylated NHIS, SHIS and bulk sweeteners

In some embodiments, the MRP, the sweetener, or both are present in the MRP-containing composition in an amount of 0.001-99 wt %, 0.001-75 wt %, 0.001-50 wt%, 0.001-25 wt%, 0.001-10 wt %, 0.001-5 wt %, 0.001-2 wt %, 0.001-1 wt %, 0.001-0.1 wt %, 0.001-0.01 wt %, 0.01-99 wt %, 0.01-75 wt %, 0.01-50 wt%, 0.01-25 wt%., 0.01-10 wt %, 0.01-5 wt %, 0.01-2 wt %, 0.01-1 wt %, 0.1-99 wt %, 0.1-75 wt %, 0.1 wt-50 wt%, 0.1-25 wt%, 0.1-10 wt %, 0.1-5 wt %, 0.1-2 wt %, 0.1-1 wt %, 0.1-0.5 wt %, 1-99 wt %, 1-75 wt %, 1-50 wt%, 1-25 wt%, 1-10 wt %, 1-5 wt %, 5-99 wt %, 5-75 wt %, 5-50 wt%, 5-25 wt%, 5-10 wt %, 10-99 wt %, 10-75 wt %, 10-50 wt%, 10-25 wt%, 10-15 wt %, 20-99 wt %, 20-75 wt %, 20-50 wt%, 30-99 wt %, 30-75 wt %, 30-50 wt%, 40-99 wt %, 40-75 wt %, 40-50 wt%, 50-99 wt %, 50-75 wt %, 60-99 wt %, 60-75 wt %, 70-99 wt %, 70-75 wt %, 80-99 wt %, 80-90 wt %, or 90-99 wt% of the MRP-containing composition.

There are four conventional basic tastes: sweet, sour salty, and bitter. Discovery of taste receptors specific for umami made academic society accepted umami as basic taste. However, it is still in dispute on how to categorize new tastes such as kukumi, fat, metallic and carbohydrate tastes. The inventor surprisingly found that non-volatile substances, such as sugar, sodium glutamate, carbohydrate, salt etc., can be carried by oral aerosol into nasal cavity, and that nasal cavity may play an important role for sensing the quality of food and beverage.

In the past, sensation and perception of umami, kukumi, carbohydrate and fat by single compound was measured without considering evolution of human’s adoption to these tastes. The inventors believe all these new tastes are evolving sensations based on a many factors, such as the cooking and/or fermentation conditions for food/beverage, the temperature at which the food/beverage is served, and enzyme activity in tester’s saliva, especially the activity of those enzymes that may hydrolyze macronutrients such as meat, fat, and starch. Therefore, it is necessary to consider the overall sensation and perception of meat, fat and starch. Protein in meat could be hydrolyzed into certain sweet amino acid, fat could be hydrolyzed into glycerol, starch could be hydrolyzed into mono and oligosaccharides. So taste wise, perception of meat, fat and starch may be classified as sweet taste. However, human overall perception of starch, meat and fat are obtained mainly from cooked foods, it is less meaningful to talk about taste only without taking aroma and mouthfeel into consideration. Retronasal cavity was evolved after human mastered the technology to manipulate fire. Human is the only species on earth to use fire. Framing and livestock domestication allowed humans to substantiaHy augment the food they obtained from hunting and gathering. Humans also know how to obtain salt around 8,000 years ago. So evolution of retronasal cavity was accelerated after humans mastered the knowledge of cooking with fire, domesticating plants and animals, and adding salt to food. The evolved retronasal cavity plays the important role to identify the hedonic level of foods which could bring the pleasure and activate the rewarding system.

The inventors found that retronasal cavity is the first place for humans to integrate the overall taste, mouthfeel and aroma originated from food and beverage. The overall perception of starch, meat and fat are integrated flavor. Starch, meat and fat have less taste but significant sensation of mouthfeel with mailard reaction flavors. Naturally, tastes of these substances should not be taken as basic taste, but should be classified as retronasal sensation and perception, neither basic tastes in mouth cavity, nor alimentary tastes in gastrointestinal tract. Umami taste should not be taken as basic taste but should be re-categorized as a sub-taste of retronasal sensation. Due to differences in biology, mechanism and function of sensation, retronasal sensation deserves an independent position from oral sensation and orthonasal olfactory sensation. So overall oral and olfactory sensation could be classified as 1) basic tastes including sweet, sour, bitter, salt; 2) supplementary cooked sensation or retronasal sensations including umami, kukumi, starch, fat, metallic sensation etc., and 3) orthonasal olfactory smell. Since retronasal cavity plays important role in sensing basic tastes, retronasal cavity could be taken as extended or supplementary areas for basic tastes in oral cavity. An embodiment of sweetening or flavor composition comprises one or more substances selected from high intensity sweeteners, hydrolyzed starch, starch, amino acid, hydrolyzed protein either from plant or animal source, protein either from plant or animal source, glycerol, hydrolyzed fat, fat either from plant or animal source.

The inventors believe that perception of chemical sensation is not simply mediated by taste receptors. The inventors hypothesized that tastants could be sensed by olfactory bulb together with aroma. Olfactory bulb rates the substances in mouth by level of harmlessness, not by the taste character. So process of sensory information could be accelerated in order to get quick decision whether the food is toxic or not, eatable or not.

Unlike conventional knowledge that perception of taste sensation is resulted by unidirectional transmitted from taste receptors in mouth cavity to brain, the inventors found that expectation in brain plays important role to regulate the overall physiological condition when tasting. Therefore, it is necessary to take consumers’ expectation of time intensity profile of sugar and physiological effect of sugar such as acting rewarding system into consideration when developing sugar reduced formulation. Of course, consumers in different regions have different expectation for food and beverage based on their traditional diets and culture.

The inventors also found that perception of food and beverage is a cognitive decision and follows expected goal-direct manner. Individual sensation is defined as a human’s capability to process, interpret, encode, manipulate, and access to individual tastant’s information so as to acquire, retain, and apply experience from memory in brain quickly and successfully to assess the value of a particular tastant. Organizational overall sensation is an organization’s capability to process, interpret, encode, manipulate, and access to overall taste, mouthfeel and aroma information of a food or beverage in a purposeful, goal-directed manner, so it can increase human’s adaptive potential in the environment in which it operates.

The inventors belivev that, in addition to the hierarchical transmission of sensory information by individual sense and final integration of all senses in brain, there are more connections among all senses within organizational sensation system, such as aggregation, cross-level, and distribution etc. based on different level of cognitive decision.

Taste receptors for different tastes distributed in oral and retronasal cavity could be counted. There are limited taste receptors for high intensity sweeteners. Different type of high intensity sweeteners have different affinities to specific sweet taste receptors. Therefore, there is ceiling of saturation for sweeteners to bind these taste receptors. For example, taste of synthetic sweeteners such as sucralose, and natural sweeteners such as stevia have uplimit sweet level with good taste not more than 6 SugarE, preferably less than 5 SugarE. There is need to look for different combination of high intensity sweeteners in order to have good sweet taste profile to realize full sugar reduction for higher sweetness level such as 10 SugarE.

Perception is a kind of trained skill. Perception is a continually dynamic process of minimizing error of prediction based on experience in memory. Perception is a kind of decision-making about how to categorize the contextual sensory information, refine the difference between top-down prediction and bottom-up sensation, then form a value judgement in an integrated manner. So perception is the process that our brain is trying hard to make best guesses about the attentive causes among noisy and ambiguous sensory signals. Perception should not be taken as a given sensation presented by stimuli. In fact, brain is actively shaping the attentive sensory information by experience in mind.

Perception is not a kind of reconstruction of sensory input in brain like a mirror. Process of perception in brain contains three core elements: generating predictive model, sensing via sensory organization, and refining comparison of the precisive weighting of sensory signals with predictive model. In other words, process of perception includes three events: we already know (the prior in memory), what we should believe next (the posterior by prediction), and what we are sensing now (the likelihood by associative context). So perception is a course of prediction error minimization, rather than a result of stimulation by sensory signals initiated by stimuli.

Generation of prediction is normally highly related to extrinsic components such as visional sensation of food and beverage associated with experience in memory and priming context. Therefore, integrated formlator of ingredients and marketing department at beginning of designing formulation is very important. The precising visual information could help the consumers to make a better prediction. Meanwhile, unlike static snapshot, perception is a kind of rolling process, The relevant priming sensory information is the key to provide the cause to make the brain’s prediction more precisely. Human being is evolved to save the energy. the consistent of relevant sensory information would help to shorten the time of making judgement by brain.

So eventually, perception is an algorithm that brain is searching similarity between the input sensory signals and predictive model in memory. Memory of food is combination of declarative and non-declarative long-term memory. The process of flavor perception is similar to visual sensation. There are contextual building blocks.

For example, fruit flavors are recognition of fruits in brain, tasting fruit is holistic contextual sensation with clear sequences. Volatile substances like conventional orthonasal smell are elements for outer controur of overall flavor, and substances including non-volatile tastants like sugar provide relevant context to brain to build the confident on the prediction of flavor.

Context sensory information in food beverage provide important information to improving accuracy of flavor prediction. Context information includes relevant familiar unconscious sensory signals, sequence and speed of different sensory information. Vivid attentive context could lead to more rapid and more accurate conscious perception. Good design of a food or beverage is not to let the brain to reshape the expectation frequently but make all clues of sensory signals as close as possible to the predicted perception.

Unlike sugar, context provided to brain by high intensity sweeteners such as sucralose and stevia are incomplete, uncertain, ambiguous, which make the perceived information very difficult to be classified when compared with expected sugar-like sweet perception. For example, unexpected slow onset sweetness makes the perceiving in brain slow and difficult because it changes the predicted sensory sequence and consequence action of flavor. Quick onset is important, because the more familiar and easy to be identified and recognized unconscious and conscious signals are more distinguishable than other un-familiar, difficult ones. The perceptual prediction of a flavor provided by vision and context are able to alter sensory activity at early stage of taste processing in a way that enhances the perception. So matching the priming context of perceptual expectations improve the guess accuracy of predicted perception to actual conscious sensation. Surely, the best experience would be the sensory signals provided by stimuli could perfectly match the overall predicted perception in every aspect. Unfortunately, overwhelming unfamiliar sensory signals of high intensity sweeteners make the sugar predicted guess take long time to change itself to match the reality of sweetness perception of high intensity sweeteners, and eventually realize it is different and it is unfamiliar.

Consistence of relevant sensory information provided by stimuli is important for prediction error minimization. Sensory signals provided by high intensity sweeteners such as slow onset, bitter, metallic, lingering attributes could be classified as low estimated precision data, which have a weak effect on associating with rooted sweetness perception immersed by sugar. So modifying these unfamiliar or unpleasant attributes of high intensity sweeteners could improve the success of brain’s predicted perception.

Increasing the estimated precision of sensory signals is nothing other than making brain have no or less attention to unfamiliar signals. The current invention helps to build similarly of flavor in food and beverage compared with fresh prepared foods. It provides a solution to make the premises of predicted sugar-like sweetness perception are true and correct, then brain’s deductive prediction could be guaranteed to be correct. The current invention provides a solution to improve the authenticity of flavor including sweetness, mouthfeel and aroma in food and beverage either sweetened by conventional sweeteners or high intensity sweeteners, thus the flavor could be quickly associated with memory, recognized by brain and categorized in brain.

Plasticity of memory in brain is like water, could be formed in millions form as per wish. It is not rigid. Water-like information stored in overall body nerves could be recalled and gather together immediately to form prediction model for sensory process. Purpose of the flavor is to distinguish food. Quick recognition and fast categorization are the skills by evolution. Ambiguous information makes slow recognition which costs a lot of energy and brain is reluctant to do such job. An embodiment of a food or beverage comprises composition in current invention, which provides a method to use unconscious tastants as flavor to block and mask conscious unpleasant and influencing sensory noises to brain, thus provide brain the predicted events: what, when, how fast and in what sequence of sensory signals go to brain.

Without limited by theory, the composition in this application could surprisingly regulate the salty taste of consumable. For instance, some sports beverages contain high salts which are not palatable, composition in this invention could reduce, inhibit or mask the salty perception of overall beverage. An embodiment of composition in this application could inhibit the salty taste of consumable. An embodiment of composition in this application provides a method to reduce the salty taste of a consumable. In some embodiemtns, the method comprises the step of adding to a salt-containing consumable a composition of the present application, wherein the composition of the present application is added to a final concentration in range of 1 ppm to 10 ppm, 1 ppm to 30 ppm, 1 ppm to 100 ppm, 1 ppm to 300 ppm, 1 ppm to 1000 ppm, 1 ppm to 3000 ppm, 1 ppm to 10,000 ppm, 1 ppm to 30,000 ppm, 1 ppm to 100,000 ppm, 10 ppm to 30 ppm, 10 ppm to 100 ppm, 10 ppm to 300 ppm, 10 ppm to 1000 ppm, 10 ppm to 3000 ppm, 10 ppm to 10,000 ppm, 10 ppm to 30,000 ppm, 10 ppm to 100,000 ppm, 30 ppm to 100 ppm, 30 ppm to 300 ppm, 30 ppm to 1000 ppm, 30 ppm to 3000 ppm, 30 ppm to 10,000 ppm, 30 ppm to 30,000 ppm, 30 ppm to 100,000 ppm, 100 ppm to 300 ppm, 100 ppm to 1000 ppm, 100 ppm to 3000 ppm, 100 ppm to 10,000 ppm, 100 ppm to 30,000 ppm, 100 ppm to 100,000 ppm, 300 ppm to 1000 ppm, 300 ppm to 3000 ppm, 300 ppm to 10,000 ppm, 300 ppm to 30,000 ppm, 300 ppm to 100,000 ppm, 1000 ppm to 3000 ppm, 1000 ppm to 10,000 ppm, 1000 ppm to 30,000 ppm, 1000 ppm to 100,000 ppm, 3000 ppm to 10,000 ppm, 3000 ppm to 30,000 ppm, 3000 ppm to 100,000 ppm, 10,000 ppm to 30,000 ppm, 10,000 ppm to 100,000 ppm, or 30,000 ppm to 100,000 ppm.

In some embodiements, the flavor or sweetening composition of the preset application comprises high intensity sweeteners and glycerin, where the weight ratio of high intensity sweeteners to glycerin is in the range of 99:1 to 1:99, 90:1 to 1:99, 80:1 to 1:99, 70:1 to 1:99, 60:1 to 1:99, 50:1 to 1:99, 40:1 to 1:99, 30:1 to 1:99, 20:1 to 1:99, 10:1 to 1:99, 1:1 to 1:99, 99:1 to 1:90, 90:1 to 1:90, 80:1 to 1:90, 70:1 to 1:90, 60:1 to 1:90, 50:1 to 1:90, 40:1 to 1:90, 30:1 to 1:90, 20:1 to 1:90, 10:1 to 1:90, 1:1 to 1:90, 99:1 to 1:60, 90:1 to 1:60, 80:1 to 1:60, 70:1 to 1:60, 60:1 to 1:60, 50:1 to 1:60, 40:1 to 1:60, 30:1 to 1:60, 20:1 to 1:60, 10:1 to 1:60, 1:1 to 1:60, 99:1 to 1:30, 90:1 to 1:30, 80:1 to 1:30, 70:1 to 1:30, 60:1 to 1:30, 50:1 to 1:30, 40:1 to 1:30, 30:1 to 1:30, 20:1 to 1:30, 10:1 to 1:30, 1:1 to 1:30, 99:1 to 1:10, 90:1 to 1:10, 80:1 to 1:10, 70:1 to 1:10, 60:1 to 1:10, 50:1 to 1:10, 40:1 to 1:10, 30:1 to 1:10, 20:1 to 1:10, 10:1 to 1:10, or 1:1 to 1:10.

In some embodiments, the composition of the presenart application is a food or beverage that comprises high intensity sweeteners and glycerin, wherein the final concentration of glycerin in the food or beverage is greater than 10 ppm, 100 ppm, 1,000 ppm, 10%, 20%, 30%, 50%, or 70% by weight. In some embodiments, the food or beverage comprises glycerin at a final concentration of 1 ppm to 10 ppm, 1 ppm to 30 ppm, 1 ppm to 100 ppm, 1 ppm to 300 ppm, 1 ppm to 1000 ppm, 1 ppm to 3000 ppm, 1 ppm to 10,000 ppm, 1 ppm to 30,000 ppm, 1 ppm to 100,000 ppm, 10 ppm to 30 ppm, 10 ppm to 100 ppm, 10 ppm to 300 ppm, 10 ppm to 1000 ppm, 10 ppm to 3000 ppm, 10 ppm to 10,000 ppm, 10 ppm to 30,000 ppm, 10 ppm to 100,000 ppm, 30 ppm to 100 ppm, 30 ppm to 300 ppm, 30 ppm to 1000 ppm, 30 ppm to 3000 ppm, 30 ppm to 10,000 ppm, 30 ppm to 30,000 ppm, 30 ppm to 100,000 ppm, 100 ppm to 300 ppm, 100 ppm to 1000 ppm, 100 ppm to 3000 ppm, 100 ppm to 10,000 ppm, 100 ppm to 30,000 ppm, 100 ppm to 100,000 ppm, 300 ppm to 1000 ppm, 300 ppm to 3000 ppm, 300 ppm to 10,000 ppm, 300 ppm to 30,000 ppm, 300 ppm to 100,000 ppm, 1000 ppm to 3000 ppm, 1000 ppm to 10,000 ppm, 1000 ppm to 30,000 ppm, 1000 ppm to 100,000 ppm, 3000 ppm to 10,000 ppm, 3000 ppm to 30,000 ppm, 3000 ppm to 100,000 ppm, 10,000 ppm to 30,000 ppm, 10,000 ppm to 100,000 ppm, or 30,000 ppm to 100,000 ppm.

In some embodiments, the composition of the presenart application is a food or beverage that comprises one or more retronasal non-volatile substances, wherein the amount of retronasal non-volatile substances is sufficient to have impact on overall intensity of flavor of the food or beverage, wherein clip-nose on and off test shows flavor intensity is increased by 0.5%, 1%, 2%, or 10%.

### III. Consumable Products of the Present Application

The compositions and methods described herein are useful in a wide range of consumable products. A non-limiting outline of products for application of the compositions described herein includes the following:
1 Dairy Products
   1.1 Milk and dairy - based drinks
      Milk and buttermilk
      Buttermilk (plain)
      Dairy based drinks, flavored and/or fermented
   1.2 Fermented, renneted milk products (excluding drinks)
   1.3 Condensed milk and analogues
      Condensed milk (plain)
      Beverage whiteners
   1.4 Cream (plain) and similar products
      Pasteurized cream
      Sterilized, UHT, whipping or whipped and reduced-fat creams
      Clotted cream
      Cream analogues
   1.5 Milk or cream powders
      Milk or cream powders
      Milk or cream powders analogues
   1.6 Cheese
      Unripened cheese
      Ripened cheese
      Whey cheese
      Processed cheese
      Cheese analogues
   1.7 Dairy-based desserts (e.g., ice cream, ice milk, pudding, fruit or flavored yogurt)
   1.8 Whey and whey products, excluding whey cheese
2 Fats and oils and fat emulsions (type water-in-oil)
   2.1 Fats and oils essentially free from water
   2.2 Fat emulsions, water-in-oil
   2.3 Fat emulsions other than 2.2, including mixed and/or flavored products based on fat emulsions.
   2.4 Fat-based desserts (excluding dairy-based desserts)
3 Edible ices, including sherbet and sorbet
4 Fruits and vegetables (including mushrooms and fungi, roots and tubers, pulses and legumes) and nuts and seeds
   4.1 Fruit
      4.1.1 Fresh fruit
         Untreated fruit
         Surface - treated fruit
         Peeled or cut fruit
      4.1.2 Processed fruit
         Frozen fruit
         Dried fruit
         Fruit in vinegar, oil or brine
         Canned or bottled (pasteurized) fruit
         Jams, jellies and marmalades
         Fruit - based spread
         Candied fruit
         Fruit preparations, including pulp and fruit toppings
         Fruit-based desserts, including fruit-flavored water-based desserts Fermented fruit products
         Fruit fillings for pastries
         Cooked or fried fruits
   4.2 Vegetables (including mushrooms and fungi, roots and tubers, pulses and legumes) and nuts and seeds
      4.2.1 Fresh vegetables
         Untreated vegetables
         Surface treated vegetables
         Peeled or cut vegetables
      4.2.2 Processed vegetable and nuts and seeds
         Frozen vegetable
         Dried vegetables
         Vegetables in vinegar, oil or brine
         Canned or bottled (pasteurized) vegetables
         Vegetable, nut and seed purees and spreads
         Vegetable, nut and seed pulps and preparations
         Fermented vegetable products
         Cooked or fried vegetables
5 Confectionery
   5.1 Cocoa products and chocolate products, including imitations and chocolate substitutes
      Cocoa mixes (powder and syrups)
      Cocoa based spreads, including fillings
      Cocoa and chocolate products (e.g., milk chocolate bars, chocolate flakes, white chocolate)
      Imitation chocolate and chocolate substitute products
   5.2 Sugar-based confectionery other than 5.1, 5.3 and 5.4, including hard and soft candy and nougats
   5.3 Chewing gum
   5.4 Decorations (e.g., for fine bakery wares), toppings (non-fruit) and sweet sauces
6 Cereals and cereal products, including flours and starches from roots and tubers, and pulses and legumes, excluding bakery wares
   Whole, broken or flaked grain, including rice
   Flours and starches
   Breakfast cereals, including rolled oats
   Pastas and noodles
   Cereals and starch-based desserts (e.g., rice pudding, tapioca pudding) Batters (e.g., for fish or poultry)
7 Bakery wares
   7.1 Bread and ordinary bakery wares
      Breads and rolls
      Crackers, excluding sweet crackers
      Other ordinary bakery products (e.g., bagels, pitta, English muffins) Bread- type products, including bread stuffing and breadcrumbs
   7.2 Fine bakery wares
      Cakes, cookies and pies (e.g., fruit-filled or custard types)
      Other fine bakery products (e.g., doughnuts, sweet rolls, scones and muffins)
      Mixes for fine bakery wares (e.g., cakes, pancakes)
8 Meat and meat products, including poultry and game
   8.1 Fresh meat, poultry and game
      Fresh meat, poultry and game, whole pieces or cuts
      Fresh meat, poultry and game, comminuted
   8.2 Processed meat, poultry and game products in whole pieces or cuts
   8.3 Processed comminuted meat, poultry and game products
   8.4 Edible casings (e.g., sausage casings)
9 Fish and fish products, including mollusks, crustaceans and echinoderms
   9.1 Fish and fish products
   9.2 Processed fish and fish products
   9.3 Semi- preserved fish and fish products
   9.4 Fully preserved fish and fish products
10 Eggs and egg products
   10.1 Fresh eggs
   10.2 Egg products
   10.3 Preserved eggs
   10.4 Egg-based desserts
11 Sweeteners, including honey
   11.1 White and semi-white sugar (sucrose or sacharose), fructose, glucose (dextrose), xylose, sugar solutions and syrups, and (partially) inverted sugars, including molasses, treacle and sugar toppings.
   11.2 Other sugar and syrups (e.g., brown sugar, maple syrup)
   11.3 Honey
   11.4 Table - top sweeteners, including those containing high-intensity sweeteners, other than 11.1-11.3
12 Salt, spices, soups, sauces, salads, protein products, etc.
   12.1 Salt
   12.2 Herbs, spices, seasonings (including salt substitutes) and condiments
   12.3 Vinegars
   12.4 Mustards
   12.5 Soups and broths
      Ready-to-eat soups and broths, including canned, bottled and frozen
      Mixes for soups and broths
   12.6 Sauces and similar products
      Emulsified sauces (e.g., mayonnaise, salad dressing)
      Non-emulsified sauces (e.g., ketchup, cheese sauce, cream sauce, brown gravy)
      Mixes for sauces and gravies
   12.7 Salads (e.g., macaroni salad, potato salad) and sandwich spreads (excluding cocoa- and nut- based spreads)
   12.8 Yeast
   12.9 Protein products
13 Foodstuffs intended for particular nutritional uses
   13.1 Infant formulae and follow-up formulae
   13.2 Foods for young children (weaning food)
   13.3 Diabetic foods intended for special medical purposes
   13.4 Diabetic formulae for slimming purposes and weight reduction
   13.5 Diabetic foods other than 13.1-13.4
   13.6 Food supplements
14 Beverage excluding dairy products
   14.1 Non-alcoholic (“soft”) beverages
      14.1.1 Waters
         Natural mineral waters and source waters
         Table waters and soda waters
      14.1.2 Fruit and vegetable juices
         Canned or bottled (pasteurized) fruit juice
         Canned or bottled (pasteurized) vegetable juice
         Concentrates (liquid or solid) for fruit juice
         Concentrates (liquid or solid) for vegetable juice
      14.1.3 Fruit and vegetable nectars
         Canned or bottled (pasteurized) fruit nectar
         Canned or bottled (pasteurized) vegetable nectar
         Concentrate (liquid or solid) for fruit nectar
         Concentrate (liquid or solid) for vegetable nectar
      14.1.4 Water-based flavored drinks, including ‘sport’ or ‘electrolyte” drinks
         Carbonated drinks
         Non-carbonated drinks, including punches
         Concentrates (liquid or solid) for drinks
      14.1.15 Coffee, coffee substitutes, tea, herbal infusions and other hot cereal beverages, excluding cocoa
   14.2 Alcoholic beverages, including alcohol-free and low-alcoholic counterparts
      14.2.1 Beer or malt beverage
      14.2.2 Cider and perry
      14.2.3 Wines
         Still wine
         Sparking and semi-sparkling wines
         Fortified wine and liquor wine
         Aromatized wine
      14.2.4 Fruit wine
      14.2.5 Mead
      14.2.6 Spirituous beverages
         Spirituous beverage containing at least 15% alcohol
         Spirituous beverage containing less than 15% alcohol
15 Ready-to-eat savories
   Snacks, potato-, cereal-, flour-, or starch-based (from roots and tubers, pulses and legumes)
   Processed nuts, including coated nuts and nut mixtures (with e.g., dried fruit)
16 Composite foods (e.g., casseroles, meat pies, mincemeat) - foods that could not be placed in categories 1-15.

In one aspect, the present application provides an orally consumable product comprising the composition of the present application described herein. The term “consumables”, as used herein, refers to substances which are contacted with the mouths of people or animals, including substances, which are taken into and subsequently ejected from the mouth, substances which are drunk, eaten, swallowed or otherwise ingested, and are safe for human or animal consumption when used in a generally acceptable range.

The compositions of the present application can be added to an orally consumable product to provide a sweetened product or a flavored product. The compositions of the present application can be incorporated into any oral consumable product, including but not limited to, for example, beverages and beverage products, food products or foodstuffs (e.g., confections, condiments, baked goods, cereal compositions, dairy products, chewing compositions, and tabletop sweetener compositions), pharmaceutical compositions, smoking compositions, oral hygiene compositions, dental compositions, and the like. Consumables can be sweetened or unsweetened. Consumables employing the compositions of the present application are also suitable for use in processed agricultural products, livestock products or seafood; processed meat products such as sausage and the like; retort food products, pickles, preserves boiled in soy sauce, delicacies, side dishes; soups; snacks, such as potato chips, cookies, or the like; as shredded filler, leaf, stem, stalk, homogenized leaf cured and animal feed.

### A. Beverages and Beverage Products

In some embodiments, a beverage or beverage product comprises a composition of the present application. The beverage may be sweetened or unsweetened. The composition of the present application, or sweetener composition comprising the same, may be added to a beverage to sweeten the beverage or to regulate a cooling effect.

A “beverage” or “beverage product,” is used herein with reference to a ready-to-drink beverage, beverage concentrate, beverage syrup, or powdered beverage. Suitable ready-to-drink beverages include carbonated and non-carbonated beverages. Carbonated beverages include, but are not limited to, frozen carbonated beverages, enhanced sparkling beverages, cola, fruit-flavored sparkling beverages (e.g., lemon-lime, orange, grape, strawberry and pineapple), ginger-ale, soft drinks and root beer. Non-carbonated beverages include, but are not limited to, fruit juice, fruit-flavored juice, juice drinks, nectars, vegetable juice, vegetable-flavored juice, sports drinks, energy drinks, enhanced water drinks, enhanced water with vitamins, near water drinks (e.g., water with natural or synthetic flavorants), coconut water, tea type drinks (e.g., black tea, green tea, red tea, oolong tea), coffee, cocoa drink, broths, beverages comprising milk components (e.g., milk beverages, coffee comprising milk components, cafe au lait, milk tea, fruit milk beverages), beverages comprising cereal extracts, and smoothies. Beverages may be frozen, semi-frozen (“slush”), non-frozen, ready-to-drink, concentrated (powdered, frozen, or syrup), dairy, non-dairy, probiotics, prebiotics, herbal, non-herbal, caffeinated, non-caffeinated, alcoholic, non-alcoholic, flavored, non-flavored, vegetable-based, fruit-based, root/tuber/corm-based, nut-based, other plant-based, cola-based, chocolate-based, meat-based, seafood-based, other animal-based, algae-based, calorie enhanced, calorie-reduced, and calorie-free.

The resulting beverages may be dispensed in open containers, cans, bottles or other packaging. Such beverages and beverage preparations can be in ready-to-drink, ready-to-cook, ready-to-mix, raw, or ingredient form and can use the composition as a sole sweetener or as a co-sweetener.

A significant challenge in the beverage industry is to preserve flavor in drinks. Normally, essential oils and their fractions are used as key flavors. They are prone to be oxidized to create unpleasant flavor(s) or the components easily evaporate to cause the food or beverage to lose their initial designed flavors as they sit on shelves. The embodiments herein provide new methods and compositions to overcome those disadvantages and provide new solutions to the food and flavor industry.

Compared with conventional flavors, which are mainly preserved in different oils or oil soluble solvents, the present embodiments provide new methods to provide water soluble solutions, syrups and powders for flavoring agents.

Compared to conventional isolated flavors, often as extracts from plant or animal sources, which are not always compatible for top note flavor and/or taste when sugar replacement sweeteners are added, the current embodiments provide new types of combined multi components which are compatible for a designed flavor.

The embodiments surprisingly create sugar reduced sweeteners which have better taste than sugar including, for example, sweetening agents from plants, such as Stevia, sweet tea, monk fruit, licorice etc., as well as synthetic sweeteners, such as sucralose.

Beverage concentrates and beverage syrups can be prepared with an initial volume of liquid matrix (e.g., water) and the desired beverage ingredients. Full strength beverages are then prepared by adding further volumes of water. Powdered beverages are prepared by dry-mixing all of the beverage ingredients in the absence of a liquid matrix. Full strength beverages are then prepared by adding the full volume of water.

Beverages comprise a matrix, i.e., the basic ingredient in which the ingredients - including the compositions of the present application - are dissolved. In one embodiment, a beverage comprises water of beverage quality as the matrix, such as, for example deionized water, distilled water, reverse osmosis water, carbon-treated water, purified water, demineralized water or combinations thereof, can be used. Additional suitable matrices include, but are not limited to phosphoric acid, phosphate buffer, citric acid, citrate buffer and carbon-treated water.

In some embodiments, the beverage or beverage product comprises one or more NHIS, one or more derivatives of NHIS, and/or one or more cooling agents, individually or collectively, at a final concentration ranging from 1 ppm to 15,000 ppm, from 1 ppm to 10,000 ppm, from 1 ppm to 5,000 ppm, from 10 ppm to 1,000 ppm, from 50 ppm to 900 ppm, from 50 ppm to 600 ppm, from 50 ppm to 500 ppm, from 50 ppm to 400 ppm, from 50 ppm to 300 ppm, from 50 ppm to 200 ppm, from 100 ppm to 600 ppm, from 100 ppm to 500 ppm, from 100 ppm to 400 ppm, from 100 ppm to 300 ppm, from 100 ppm to 200 ppm, from 125 ppm to 600 ppm, from 125 ppm to 500 ppm, from 125 ppm to 400 ppm, from 125 ppm to 300 ppm, from 125 ppm to 200 ppm, from 150 ppm to 600 ppm, from 150 ppm to 500 ppm, from 150 ppm to 500 ppm, from 150 ppm to 400 ppm, from 150 ppm to 300 ppm, from 150 ppm to 200 ppm, from 200 ppm to 600 ppm, from 200 ppm to 500 ppm, from 200 ppm to 400 ppm, from 200 ppm to 300 ppm, from 300 ppm to 600 ppm, from 300 ppm to 500 ppm, from 300 ppm to 400 ppm, from 400 ppm to 600 ppm, from 500 ppm to 600 ppm, from 20 ppm to 200 ppm, from 20 ppm to 180 ppm, from 20 ppm to 160 ppm, from 20 ppm to 140 ppm, from 20 ppm to 120 ppm, from 20 ppm to 100 ppm, from 20 ppm to 80 ppm, from 20 ppm to 60 ppm, from 20 ppm to 40 ppm, from 40 ppm to 150 ppm, from 40 ppm to 130 ppm, from 40 ppm to 100 ppm, from 40 ppm to 90 ppm, from 40 ppm to 70 ppm, from 40 ppm to 50 ppm, from 20 ppm to 100 ppm, from 40 ppm to 100 ppm, from 50 ppm to 100 ppm, from 60 ppm to 100 ppm, from 80 ppm to 100 ppm, from 5 ppm to 100 ppm, from 5 ppm to 95 ppm, from 5 ppm to 90 ppm, from 5 ppm to 85 ppm, from 5 ppm to 80 ppm, from 5 ppm to 75 ppm, from 5 ppm to 70 ppm, from 5 ppm to 65 ppm, from 5 ppm to 60 ppm, from 5 ppm to 55 ppm, from 5 ppm to 50 ppm, from 5 ppm to 45 ppm, from 5 ppm to 40 ppm, from 5 ppm to 35 ppm, from 5 ppm to 30 ppm, from 5 ppm to 25 ppm, from 5 ppm to 20 ppm, from 5 ppm to 15 ppm, or from 5 ppm to 10 ppm.

### B. Confections

In some embodiments, the consumable product comprising one or more NHIS, one or more derivatives of NHIS, and/or one or more cooling agents is a confection..

In some embodiments of the present application, the confection may be a bakery product, such as a pastry, Bavarian cream, blancmange, cake, brownie, cookie, mousse and the like; a dessert, such as yogurt, a jelly, a drinkable jelly, a pudding; a sweetened food product eaten at tea time or following meals; a frozen food; a cold confection, such as ice, ice milk, lacto-ice and the like (food products in which sweeteners and various other types of raw materials are added to milk products, and the resulting mixture is agitated and frozen); ice confections, such as sherbets, dessert ices and the like (food products in which various other types of raw materials are added to a sugary liquid, and the resulting mixture is agitated and frozen); general confections, e.g., baked confections or steamed confections such as crackers, biscuits, buns with bean-jam filling, halvah, alfajor, and the like; rice cakes and snacks; table top products; general sugar confections such as chewing gum (e.g., including compositions which comprise a substantially water-insoluble, chewable gum base, such as chicle or substitutes thereof, including jetulong, guttakay rubber or certain comestible natural synthetic resins or waxes), hard candy, soft candy, mints, nougat candy, jelly beans, fudge, toffee, taffy, Swiss milk tablet, licorice candy, chocolates, gelatin candies, marshmallow, marzipan, divinity, cotton candy, and the like; sauces including fruit flavored sauces, chocolate sauces and the like; edible gels; cremes including butter cremes, flour pastes, whipped cream and the like; jams including strawberry jam, marmalade and the like; and breads including sweet breads and the like or other starch products, or combinations thereof.

In some embodiments, the confection comprises one or more NHIS, one or more derivatives of NHIS, and/or one or more cooling agents, individually or collectively, at a final weight percentage range from 0.001 wt % to 99 wt %, 0.001 wt % to 75 wt %, 0.001 wt % to 50 wt%, 0.001 wt % to 25 wt%, 0.001 wt % to 10 wt %, 0.001 wt % to 5 wt %, 0.001 wt % to 2 wt %, 0.001 wt % to 1 wt %, 0.001 wt % to 0.1 wt %, 0.001 wt % to 0.01 wt %, 0.01 wt % to 99 wt %, 0.01 wt % to 75 wt %, 0.01 wt % to 50 wt%, 0.01 wt % to 25 wt%., 0.01 wt % to 10 wt %, 0.01 wt % to 5 wt %, 0.01 wt % to 2 wt %, 0.01 wt % to 1 wt %, 0.1 wt % to 99 wt %, 0.1 wt % to 75 wt %, 0.1 wt % to 50 wt%, 0.1 wt % to 25 wt%, 0.1 wt % to 10 wt %, 0.1 wt % to 5 wt %, 0.1 wt % to 2 wt %, 0.1 wt % to 1 wt %, 0.1 wt % to 0.5 wt %, 1 wt % to 99 wt %, 1 wt % to 75 wt %, 1 wt % to 50 wt%, 1 wt % to 25 wt%, 1 wt % to 10 wt %, 1 wt % to 5 wt %, 5 wt % to 99 wt %, 5 wt % to 75 wt %, 5 wt % to 50 wt%, 5 wt % to 25 wt%, 5 wt % to 10 wt %, 10 wt % to 99 wt %, 10 wt % to 75 wt %, 10 wt % to 50 wt%, 10 wt % to 25 wt%, 10 wt % to 15 wt %, 20 wt % to 99 wt %, 20 wt % to 75 wt %, 20 wt % to 50 wt%, 30 wt % to 99 wt %, 30 wt % to 75 wt %, 30 wt % to 50 wt%, 40 wt % to 99 wt %, 40 wt % to 75 wt %, 40 wt % to 50 wt%, 50 wt % to 99 wt %, 50 wt % to 75 wt %, 60 wt % to 99 wt %, 60 wt % to 75 wt %, 70 wt % to 99 wt %, 70 wt % to 75 wt %, 80 wt % to 99 wt %, 80 wt % to 90 wt %, or 90 wt % to 99 wt% of the confection.

### C. Condiments

In some embodiments, the consumable product comprising one or more NHIS, one or more derivatives of NHIS, and/or one or more cooling agents is a condiment. Condiments, as used herein, are compositions used to regulate the flavor of a food or beverage. Non-limiting examples of condiments include ketchup (catsup); mustard; barbecue sauce; butter; chili sauce; chutney; cocktail sauce; curry; dips; fish sauce; horseradish; hot sauce; jellies, jams, marmalades, or preserves; mayonnaise; peanut butter; relish; remoulade; salad dressings (e.g., oil and vinegar, Caesar, French, ranch, bleu cheese, Russian, Thousand Island, Italian, and balsamic vinaigrette), salsa; sauerkraut; soy sauce; steak sauce; syrups; tartar sauce; and Worcestershire sauce.

Condiment bases generally comprise a mixture of different ingredients, non-limiting examples of which include vehicles (e.g., water and vinegar); spices or seasonings (e.g., salt, pepper, garlic, mustard seed, onion, paprika, turmeric, or combinations thereof); fruits, vegetables, or their products (e.g., tomatoes or tomato-based products (paste, puree), fruit juices, fruit juice peels, or combinations thereof); oils or oil emulsions, particularly vegetable oils; thickeners (e.g., xanthan gum, food starch, other hydrocolloids, or combinations thereof); and emulsifying agents (e.g., egg yolk solids, protein, gum arabic, carob bean gum, guar gum, gum karaya, gum tragacanth, carageenan, pectin, propylene glycol esters of alginic acid, sodium carboxymethyl-cellulose, polysorbates, or combinations thereof). Recipes for condiment bases and methods of making condiment bases are well known to those of ordinary skill in the art.

The condiment of the present applicaiton optionally may include synthetic high-potency sweeteners, bulk sweeteners, pH modifying agents (e.g., lactic acid, citric acid, phosphoric acid, hydrochloric acid, acetic acid, or combinations thereof), fillers, functional agents (e.g., pharmaceutical agents, nutrients, or components of a food or plant), flavoring agents, colorings, or combinations thereof.

In some embodiments, the condiment comprises one or more NHIS, one or more derivatives of NHIS, and/or one or more cooling agents, individually or collectively, at a final weight percentage range from 0.001 wt % to 99 wt %, 0.001 wt % to 75 wt %, 0.001 wt % to 50 wt%, 0.001 wt % to 25 wt%, 0.001 wt % to 10 wt %, 0.001 wt % to 5 wt %, 0.001 wt % to 2 wt %, 0.001 wt % to 1 wt %, 0.001 wt % to 0.1 wt %, 0.001 wt % to 0.01 wt %, 0.01 wt % to 99 wt %, 0.01 wt % to 75 wt %, 0.01 wt % to 50 wt%, 0.01 wt % to 25 wt%., 0.01 wt % to 10 wt %, 0.01 wt % to 5 wt %, 0.01 wt % to 2 wt %, 0.01 wt % to 1 wt %, 0.1 wt % to 99 wt %, 0.1 wt % to 75 wt %, 0.1 wt % to 50 wt%, 0.1 wt % to 25 wt%, 0.1 wt % to 10 wt %, 0.1 wt % to 5 wt %, 0.1 wt % to 2 wt %, 0.1 wt % to 1 wt %, 0.1 wt % to 0.5 wt %, 1 wt % to 99 wt %, 1 wt % to 75 wt %, 1 wt % to 50 wt%, 1 wt % to 25 wt%, 1 wt % to 10 wt %, 1 wt % to 5 wt %, 5 wt % to 99 wt %, 5 wt % to 75 wt %, 5 wt % to 50 wt%, 5 wt % to 25 wt%, 5 wt % to 10 wt %, 10 wt % to 99 wt %, 10 wt % to 75 wt %, 10 wt % to 50 wt%, 10 wt % to 25 wt%, 10 wt % to 15 wt %, 20 wt % to 99 wt %, 20 wt % to 75 wt %, 20 wt % to 50 wt%, 30 wt % to 99 wt %, 30 wt % to 75 wt %, 30 wt % to 50 wt%, 40 wt % to 99 wt %, 40 wt % to 75 wt %, 40 wt % to 50 wt%, 50 wt % to 99 wt %, 50 wt % to 75 wt %, 60 wt % to 99 wt %, 60 wt % to 75 wt %, 70 wt % to 99 wt %, 70 wt % to 75 wt %, 80 wt % to 99 wt %, 80 wt % to 90 wt %, 90 wt % to 99 wt%, or a weight concentration range defined by any two of the aforementioned weight percentages in this paragraph.

### D. Dairy Products

A wide variety of dairy products can be made using the composition of the present invention. Such products include without limitation, milk, whole milk, buttermilk, skim milk, infant formula, condensed milk, dried milk, evaporated milk, fermented milk, butter, clarified butter, cottage cheese, cream cheese, and various types of cheese.

In some embodiments, the dairy product comprises one or more NHIS, one or more derivatives of NHIS, and/or one or more cooling agents, individually or collectively, at a weight percentage range from 0.001 wt % to 99 wt %, 0.001 wt % to 75 wt %, 0.001 wt % to 50 wt%, 0.001 wt % to 25 wt%, 0.001 wt % to 10 wt %, 0.001 wt % to 5 wt %, 0.001 wt % to 2 wt %, 0.001 wt % to 1 wt %, 0.001 wt % to 0.1 wt %, 0.001 wt % to 0.01 wt %, 0.01 wt % to 99 wt %, 0.01 wt % to 75 wt %, 0.01 wt % to 50 wt%, 0.01 wt % to 25 wt%., 0.01 wt % to 10 wt %, 0.01 wt % to 5 wt %, 0.01 wt % to 2 wt %, 0.01 wt % to 1 wt %, 0.1 wt % to 99 wt %, 0.1 wt % to 75 wt %, 0.1 wt % to 50 wt%, 0.1 wt % to 25 wt%, 0.1 wt % to 10 wt %, 0.1 wt % to 5 wt %, 0.1 wt % to 2 wt %, 0.1 wt % to 1 wt %, 0.1 wt % to 0.5 wt %, 1 wt % to 99 wt %, 1 wt % to 75 wt %, 1 wt % to 50 wt%, 1 wt % to 25 wt%, 1 wt % to 10 wt %, 1 wt % to 5 wt %, 5 wt % to 99 wt %, 5 wt % to 75 wt %, 5 wt % to 50 wt%, 5 wt % to 25 wt%, 5 wt % to 10 wt %, 10 wt % to 99 wt %, 10 wt % to 75 wt %, 10 wt % to 50 wt%, 10 wt % to 25 wt%, 10 wt % to 15 wt %, 20 wt % to 99 wt %, 20 wt % to 75 wt %, 20 wt % to 50 wt%, 30 wt % to 99 wt %, 30 wt % to 75 wt %, 30 wt % to 50 wt%, 40 wt % to 99 wt %, 40 wt % to 75 wt %, 40 wt % to 50 wt%, 50 wt % to 99 wt %, 50 wt % to 75 wt %, 60 wt % to 99 wt %, 60 wt % to 75 wt %, 70 wt % to 99 wt %, 70 wt % to 75 wt %, 80 wt % to 99 wt %, 80 wt % to 90 wt %, or 90 wt % to 99 wt% of the dairy product.

Alternatively, in any of the liquid dairy compositions described herein, the one or more NHIS, one or more derivatives of NHIS, and/or one or more cooling agents may be present in the liquid dairy composition, individually or collectively at a final concentration ranging from 1 ppm to 15,000 ppm, from 1 ppm to 10,000 ppm, from 1 ppm to 5,000 ppm, from 10 ppm to 1,000 ppm, from 50 ppm to 900 ppm, from 50 ppm to 600 ppm, from 50 ppm to 500 ppm, from 50 ppm to 400 ppm, from 50 ppm to 300 ppm, from 50 ppm to 200 ppm, from 100 ppm to 600 ppm, from 100 ppm to 500 ppm, from 100 ppm to 400 ppm, from 100 ppm to 300 ppm, from 100 ppm to 200 ppm, from 125 ppm to 600 ppm, from 125 ppm to 500 ppm, from 125 ppm to 400 ppm, from 125 ppm to 300 ppm, from 125 ppm to 200 ppm, from 150 ppm to 600 ppm, from 150 ppm to 500 ppm, from 150 ppm to 500 ppm, from 150 ppm to 400 ppm, from 150 ppm to 300 ppm, from 150 ppm to 200 ppm, from 200 ppm to 600 ppm, from 200 ppm to 500 ppm, from 200 ppm to 400 ppm, from 200 ppm to 300 ppm, from 300 ppm to 600 ppm, from 300 ppm to 500 ppm, from 300 ppm to 400 ppm, from 400 ppm to 600 ppm, from 500 ppm to 600 ppm, from 20 ppm to 200 ppm, from 20 ppm to 180 ppm, from 20 ppm to 160 ppm, from 20 ppm to 140 ppm, from 20 ppm to 120 ppm, from 20 ppm to 100 ppm, from 20 ppm to 80 ppm, from 20 ppm to 60 ppm, from 20 ppm to 40 ppm, from 40 ppm to 150 ppm, from 40 ppm to 130 ppm, from 40 ppm to 100 ppm, from 40 ppm to 90 ppm, from 40 ppm to 70 ppm, from 40 ppm to 50 ppm, from 20 ppm to 100 ppm, from 40 ppm to 100 ppm, from 50 ppm to 100 ppm, from 60 ppm to 100 ppm, from 80 ppm to 100 ppm, from 5 ppm to 100 ppm, from 5 ppm to 95 ppm, from 5 ppm to 90 ppm, from 5 ppm to 85 ppm, from 5 ppm to 80 ppm, from 5 ppm to 75 ppm, from 5 ppm to 70 ppm, from 5 ppm to 65 ppm, from 5 ppm to 60 ppm, from 5 ppm to 55 ppm, from 5 ppm to 50 ppm, from 5 ppm to 45 ppm, from 5 ppm to 40 ppm, from 5 ppm to 35 ppm, from 5 ppm to 30 ppm, from 5 ppm to 25 ppm, from 5 ppm to 20 ppm, from 5 ppm to 15 ppm, or from 5 ppm to 10 ppm.

### E. Cereal Compositions

In some embodiments, the consumable product comprising one or more NHIS, one or more derivatives of NHIS, and/or one or more cooling agents is a cereal composition. Cereal compositions typically are eaten either as staple foods or as snacks. Non-limiting examples of cereal compositions for use in some embodiments include ready-to-eat cereals as well as hot cereals. Ready-to-eat cereals are cereals which may be eaten without further processing (i.e., cooking) by the consumer. Examples of ready-to-eat cereals include breakfast cereals and snack bars. Breakfast cereals typically are processed to produce a shredded, flaky, puffy, or extruded form. Breakfast cereals generally are eaten cold and are often mixed with milk and/or fruit. Snack bars include, for example, energy bars, rice cakes, granola bars, and nutritional bars. Hot cereals generally are cooked, usually in either milk or water, before being eaten. Non-limiting examples of hot cereals include grits, porridge, polenta, rice, oatmeal, and rolled oats.

Cereal compositions generally comprise at least one cereal ingredient. As used herein, the term “cereal ingredient” denotes materials such as whole or part grains, whole or part seeds, and whole or part grass. Non-limiting examples of cereal ingredients for use in some embodiments include maize, wheat, rice, barley, bran, bran endosperm, bulgur, sorghums, millets, oats, rye, triticale, buckwheat, fonio, quinoa, bean, soybean, amaranth, teff, spelt, and kaniwa.

The cereal composition may include one or more NHIS, one or more derivatives of NHIS, and/or one or more cooling agents of the present application and at least one cereal ingredient. The one or more NHIS, one or more derivatives of NHIS, and/or one or more cooling agents may be added to the cereal composition in a variety of ways, such as, for example, as a coating, as a frosting, as a glaze, or as a matrix blend (i.e., added as an ingredient to the cereal formulation prior to the preparation of the final cereal product).

Accordingly, in some embodiments, one or more NHIS, one or more derivatives of NHIS, and/or one or more cooling agents of the present application may be added to the cereal composition as a matrix blend. In one embodiment, the one or more NHIS, one or more derivatives of NHIS, and/or one or more cooling agents are blended with a hot cereal prior to cooking to provide a sweetened hot cereal product. In another embodiment, the one or more NHIS, one or more derivatives of NHIS, and/or one or more cooling agents are blended with the cereal matrix before the cereal is extruded.

In some embodiments, one or more NHIS, one or more derivatives of NHIS, and/or one or more cooling agents may be present, individually or collectively are added to the cereal composition as a coating, such as, for example, in combination with food grade oil and applying the mixture onto the cereal. In a different embodiment, one or more sweetening agent(s) and the food grade oil are applied to the cereal separately, by applying either the oil or the sweetener first. Non-limiting examples of food grade oils for use some embodiments include vegetable oils such as corn oil, soybean oil, cottonseed oil, peanut oil, coconut oil, canola oil, olive oil, sesame seed oil, palm oil, palm kernel oil, or mixtures thereof. In yet another embodiment, food grade fats may be used in place of the oils, provided that the fat is melted prior to applying the fat onto the cereal.

In another embodiment, one or more NHIS, one or more derivatives of NHIS, and/or one or more cooling agents are added to the cereal composition as a glaze. Non-limiting examples of glazing agents for use in some embodiments include corn syrup, honey syrups and honey syrup solids, maple syrups and maple syrup solids, sucrose, isomalt, polydextrose, polyols, hydrogenated starch hydrolysate, aqueous solutions thereof, or mixtures thereof. In another such embodiment, the composition is formulated as a glaze by combining with a glazing agent and a food grade oil or fat and applying the mixture to the cereal. In yet another embodiment, a gum system, such as, for example, gum acacia, carboxymethyl cellulose, or algin, may be added to the glaze to provide structural support. In addition, the glaze also may include a coloring agent, and also may include a flavor.

In another embodiment, one or more NHIS, one or more derivatives of NHIS, and/or one or more cooling agents are added to the cereal composition as a frosting. In one such embodiment, the composition is combined with water and a frosting agent and then applied to the cereal. Non-limiting examples of frosting agents for use in some embodiments include maltodextrin, sucrose, starch, polyols, or mixtures thereof. The frosting also may include a food grade oil, a food grade fat, a coloring agent, and/or a flavor.

In some embodiments, the cereal composition comprises one or more NHIS, one or more derivatives of NHIS, and/or one or more cooling agents, individually or collectively, at a weight percentage range from 0.001 wt % to 99 wt %, 0.001 wt % to 75 wt %, 0.001 wt % to 50 wt%, 0.001 wt % to 25 wt%, 0.001 wt % to 10 wt %, 0.001 wt % to 5 wt %, 0.001 wt % to 2 wt %, 0.001 wt % to 1 wt %, 0.001 wt % to 0.1 wt %, 0.001 wt % to 0.01 wt %, 0.01 wt % to 99 wt %, 0.01 wt % to 75 wt %, 0.01 wt % to 50 wt%, 0.01 wt % to 25 wt%., 0.01 wt % to 10 wt %, 0.01 wt % to 5 wt %, 0.01 wt % to 2 wt %, 0.01 wt % to 1 wt %, 0.1 wt % to 99 wt %, 0.1 wt % to 75 wt %, 0.1 wt % to 50 wt%, 0.1 wt % to 25 wt%, 0.1 wt % to 10 wt %, 0.1 wt % to 5 wt %, 0.1 wt % to 2 wt %, 0.1 wt % to 1 wt %, 0.1 wt % to 0.5 wt %, 1 wt % to 99 wt %, 1 wt % to 75 wt %, 1 wt % to 50 wt%, 1 wt % to 25 wt%, 1 wt % to 10 wt %, 1 wt % to 5 wt %, 5 wt % to 99 wt %, 5 wt % to 75 wt %, 5 wt % to 50 wt%, 5 wt % to 25 wt%, 5 wt % to 10 wt %, 10 wt % to 99 wt %, 10 wt % to 75 wt %, 10 wt % to 50 wt%, 10 wt % to 25 wt%, 10 wt % to 15 wt %, 20 wt % to 99 wt %, 20 wt % to 75 wt %, 20 wt % to 50 wt%, 30 wt % to 99 wt %, 30 wt % to 75 wt %, 30 wt % to 50 wt%, 40 wt % to 99 wt %, 40 wt % to 75 wt %, 40 wt % to 50 wt%, 50 wt % to 99 wt %, 50 wt % to 75 wt %, 60 wt % to 99 wt %, 60 wt % to 75 wt %, 70 wt % to 99 wt %, 70 wt % to 75 wt %, 80 wt % to 99 wt %, 80 wt % to 90 wt %, or 90 wt % to 99 wt% of the cereal composition.

### F. Chewing Compositions

In some embodiments, the consumable product comprising one or more NHIS, one or more derivatives of NHIS, and/or one or more cooling agents is a chewing composition. The term “chewing compositions” include chewing gum compositions, chewing tobacco, smokeless tobacco, snuff, chewing gum and other compositions which are masticated and subsequently expectorated.

Chewing gum compositions generally comprise a water-soluble portion and a water-insoluble chewable gum base portion. The water soluble portion, which typically includes one or more components of the compositions of the present application, dissipates with a portion of the flavoring agent over a period of time during chewing while the insoluble gum base portion is retained in the mouth. The insoluble gum base generally determines whether a gum is considered chewing gum, bubble gum, or a functional gum.

The insoluble gum base, which is generally present in the chewing gum composition in an amount in the range of about 15 to about 35 weight percent of the chewing gum composition, generally comprises combinations of elastomers, softeners (plasticizers), emulsifiers, resins, and fillers. Such components generally are considered food grade, recognized as safe (GRA), and/or are U.S. Food and Drug Administration (FDA)-approved.

Elastomers, the primary component of the gum base, provide the rubbery, cohesive nature to gums and can include one or more natural rubbers (e.g., smoked latex, liquid latex, or guayule); natural gums (e.g., jelutong, perillo, sorva, massaranduba balata, massaranduba chocolate, nispero, rosindinha, chicle, and gutta hang kang); or synthetic elastomers (e.g., butadiene-styrene copolymers, isobutylene-isoprene copolymers, polybutadiene, polyisobutylene, and vinyl polymeric elastomers). In a particular embodiment, the elastomer is present in the gum base in an amount in the range of about 3 to about 50 weight percent of the gum base.

Resins are used to vary the firmness of the gum base and aid in softening the elastomer component of the gum base. Non-limiting examples of suitable resins include a rosin ester, a terpene resin (e.g., a terpene resin from α-pinene, β-pinene and/or D-limonene), polyvinyl acetate, polyvinyl alcohol, ethylene vinyl acetate, and vinyl acetate-vinyl laurate copolymers. Non-limiting examples of rosin esters include a glycerol ester of a partially hydrogenated rosin, a glycerol ester of a polymerized rosin, a glycerol ester of a partially dimerized rosin, a glycerol ester of rosin, a pentaerythritol ester of a partially hydrogenated rosin, a methyl ester of rosin, or a methyl ester of a partially hydrogenated rosin. In some embodiment, the resin is present in the gum base in an amount in the range of about 5 to about 75 weight percent of the gum base.

Softeners, which also are known as plasticizers, are used to modify the ease of chewing and/or mouth feel of the chewing gum composition. Generally, softeners comprise oils, fats, waxes, and emulsifiers. Non-limiting examples of oils and fats include tallow, hydrogenated tallow, large, hydrogenated or partially hydrogenated vegetable oils (e.g., soybean, canola, cottonseed, sunflower, palm, coconut, corn, safflower, or palm kernel oils), cocoa butter, glycerol monostearate, glycerol triacetate, glycerol abietate, lecithin, monoglycerides, diglycerides, triglycerides acetylated monoglycerides, and free fatty acids. Non-limiting examples of waxes include polypropylene/polyethylene/Fisher-Tropsch waxes, paraffin, and microcrystalline and natural waxes (e.g., candelilla, beeswax and carnauba). Microcrystalline waxes, especially those with a high degree of crystallinity and a high melting point, also may be considered as bodying agents or textural modifiers. In some embodiments, the softeners are present in the gum base in an amount in the range of about 0.5 to about 25 weight percent of the gum base.

Emulsifiers are used to form a uniform dispersion of the insoluble and soluble phases of the chewing gum composition and also have plasticizing properties. Suitable emulsifiers include glycerol monostearate (GMS), lecithin (phosphatidyl choline), polyglycerol polyricinoleic acid (PPGR), mono and diglycerides of fatty acids, glycerol distearate, tracetin, acetylated monoglyceride, glycerol triacetate, and magnesium stearate. In some embodiments, the emulsifiers are present in the gum base in an amount in the range of about 2 to about 30 weight percent of the gum base.

The chewing gum composition also may comprise adjuvants or fillers in either the gum base and/or the soluble portion of the chewing gum composition. Suitable adjuvants and fillers include lecithin, inulin, polydextrin, calcium carbonate, magnesium carbonate, magnesium silicate, ground limestone, aluminum hydroxide, aluminum silicate, talc, clay, alumina, titanium dioxide, and calcium phosphate. In some embodiments, lecithin can be used as an inert filler to decrease the stickiness of the chewing gum composition. In other some embodiments, lactic acid copolymers, proteins (e.g., gluten and/or zein) and/or guar can be used to create a gum that is more readily biodegradable. The adjuvants or fillers are generally present in the gum base in an amount up to about 20 weight percent of the gum base. Other optional ingredients include coloring agents, whiteners, preservatives, and flavors.

In some embodiments of the chewing gum composition, the gum base comprises about 5 to about 95 weight percent of the chewing gum composition, more desirably about 15 to about 50 weight percent of the chewing gum composition, and even more desirably from about 20 to about 30 weight percent of the chewing gum composition.

The soluble portion of the chewing gum composition may optionally include other artificial or natural sweeteners, bulk sweeteners, softeners, emulsifiers, flavoring agents, coloring agents, adjuvants, fillers, functional agents (e.g., pharmaceutical agents or nutrients), or combinations thereof. Suitable examples of softeners and emulsifiers are described above.

Bulk sweeteners include both caloric and non-caloric compounds. Non-limiting examples of bulk sweeteners include sucrose, dextrose, maltose, dextrin, dried invert sugar, fructose, high fructose corn syrup, levulose, galactose, corn syrup solids, tagatose, polyols (e.g., sorbitol, mannitol, xylitol, lactitol, erythritol, and maltitol), hydrogenated starch hydrolysates, isomalt, trehalose, or mixtures thereof. In some embodiments, the bulk sweetener is present in the chewing gum composition in an amount in the range of about 1 to about 75 weight percent of the chewing gum composition.

Flavoring agents may be used in either the insoluble gum base or soluble portion of the chewing gum composition. Such flavoring agents may be natural or artificial flavors. In some embodiments, the flavoring agent comprises an essential oil, such as an oil produced from a plant or a fruit, peppermint oil, spearmint oil, other mint oils, clove oil, cinnamon oil, oil of wintergreen, bay, thyme, cedar leaf, nutmeg, allspice, sage, mace, and almonds. In another embodiment, the flavoring agent comprises a plant extract or a fruit essence such as apple, banana, watermelon, pear, peach, grape, strawberry, raspberry, cherry, plum, pineapple, apricot, or mixtures thereof. In still another embodiment, the flavoring agent comprises a citrus flavor, such as an extract, essence, or oil of lemon, lime, orange, tangerine, grapefruit, citron, or kumquat.

In some embodiments, the chewing gum composition comprises (1) one or more NHIS, one or more derivatives of NHIS, and/or one or more cooling agents and (2) a gum base.

In some embodiments, the gum compositions comprisies one or more NHIS, one or more derivatives of NHIS, and/or one or more cooling agents, individually or collectively, at a weight percentage range from 0.001 wt % to 99 wt %, 0.001 wt % to 75 wt %, 0.001 wt % to 50 wt%, 0.001 wt % to 25 wt%, 0.001 wt % to 10 wt %, 0.001 wt % to 5 wt %, 0.001 wt % to 2 wt %, 0.001 wt % to 1 wt %, 0.001 wt % to 0.1 wt %, 0.001 wt % to 0.01 wt %, 0.01 wt % to 99 wt %, 0.01 wt % to 75 wt %, 0.01 wt % to 50 wt%, 0.01 wt % to 25 wt%., 0.01 wt % to 10 wt %, 0.01 wt % to 5 wt %, 0.01 wt % to 2 wt %, 0.01 wt % to 1 wt %, 0.1 wt % to 99 wt %, 0.1 wt % to 75 wt %, 0.1 wt % to 50 wt%, 0.1 wt % to 25 wt%, 0.1 wt % to 10 wt %, 0.1 wt % to 5 wt %, 0.1 wt % to 2 wt %, 0.1 wt % to 1 wt %, 0.1 wt % to 0.5 wt %, 1 wt % to 99 wt %, 1 wt % to 75 wt %, 1 wt % to 50 wt%, 1 wt % to 25 wt%, 1 wt % to 10 wt %, 1 wt % to 5 wt %, 5 wt % to 99 wt %, 5 wt % to 75 wt %, 5 wt % to 50 wt%, 5 wt % to 25 wt%, 5 wt % to 10 wt %, 10 wt % to 99 wt %, 10 wt % to 75 wt %, 10 wt % to 50 wt%, 10 wt % to 25 wt%, 10 wt % to 15 wt %, 20 wt % to 99 wt %, 20 wt % to 75 wt %, 20 wt % to 50 wt%, 30 wt % to 99 wt %, 30 wt % to 75 wt %, 30 wt % to 50 wt%, 40 wt % to 99 wt %, 40 wt % to 75 wt %, 40 wt % to 50 wt%, 50 wt % to 99 wt %, 50 wt % to 75 wt %, 60 wt % to 99 wt %, 60 wt % to 75 wt %, 70 wt % to 99 wt %, 70 wt % to 75 wt %, 80 wt % to 99 wt %, 80 wt % to 90 wt %, or 90 wt % to 99 wt% of the gum composition.

### G. Tabletop Sweetener Compositions

In some embodiments, the consumable product comprising one or more NHIS, one or more derivatives of NHIS, and/or one or more cooling agents is a tabletop sweetener composition. In some embodiments, the tabletop sweetener composition may further include at least one bulking agent, additive, anti-caking agent, functional ingredient or combination thereof.

Suitable “bulking agents” include, but are not limited to, maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, or mixtures thereof. Additionally, in accordance with still other embodiments of the application, granulated sugar (sucrose) or other caloric sweeteners such as crystalline fructose, other carbohydrates, or sugar alcohol can be used as a bulking agent due to their provision of good content uniformity without the addition of significant calories.

As used herein, the phrase “anti-caking agent” and “flow agent” refers to any composition which assists in content uniformity and uniform dissolution. In some embodiments, non-limiting examples of anti-caking agents include cream of tartar, aluminium silicate (Kaolin), calcium aluminium silicate, calcium carbonate, calcium silicate, magnesium carbonate, magnesium silicate, mono-, di- and tri-calcium orthophosphate, potassium aluminium silicate, silicon dioxide, sodium aluminium silicate, salts of stearic acid, microcrystalline cellulose (Avicel, FMC BioPolymer, Philadelphia, Pennsylvania), and tricalcium phosphate. In one embodiment, the anti-caking agents are present in the tabletop sweetener composition in an amount from about 0.001 to about 3 % by weight of the tabletop sweetener composition.

The tabletop sweetener compositions can be packaged in any form known in the art. Non-limiting forms include, but are not limited to, powder form, granular form, packets, tablets, sachets, pellets, cubes, solids, and liquids.

In one embodiment, the tabletop sweetener composition is a single-serving (portion control) packet comprising a dry-blend. Dry-blend formulations generally may comprise powder or granules. Although the tabletop sweetener composition may be in a packet of any size, an illustrative non-limiting example of conventional portion control tabletop sweetener packets are approximately 2.5 by 1.5 inches and hold approximately 1 gram of a sweetener composition having a sweetness equivalent to 2 teaspoons of granulated sugar (~ 8 g). The amount of an MRP composition of the present application in a dry-blend tabletop sweetener formulation can vary. In some embodiments, a dry-blend tabletop sweetener formulation may comprise a Composition of the present application in an amount from about 1 % (w/w) to about 10 % (w/w) of the tabletop sweetener composition.

Solid tabletop sweetener embodiments include cubes and tablets. A non-limiting example of conventional cubes is equivalent in size to a standard cube of granulated sugar, which is approximately 2.2 x 2.2 x 2.2 cm3 and weighs approximately 8 g. In one embodiment, a solid tabletop sweetener is in the form of a tablet or any other form known to those skilled in the art.

A tabletop sweetener composition also may be embodied in the form of a liquid, wherein the components of the compositions of the present application are combined with a liquid carrier. Suitable non-limiting examples of carrier agents for liquid tabletop sweeteners include water, alcohol, polyol, glycerin base or citric acid base dissolved in water, or mixtures thereof. The sweetness equivalent of a tabletop sweetener composition for any of the forms described herein or known in the art may be varied to obtain a desired sweetness profile. For example, a tabletop sweetener composition may have a degree of sweetness comparable to that of an equivalent amount of standard sugar. In another embodiment, the tabletop sweetener composition may comprise a sweetness of up to 100 times that of an equivalent amount of sugar. In another embodiment, the tabletop sweetener composition may comprise a sweetness of up to 90 times, 80 times, 70 times, 60 times, 50 times, 40 times, 30 times, 20 times, 10 times, 9 times, 8 times, 7 times, 6 times, 5 times, 4 times, 3 times, and 2 times that of an equivalent amount of sugar.

In some embodiments, the tabletop sweetener compositions comprises one or more NHIS, one or more derivatives of NHIS, and/or one or more cooling agents, individually or collectively, at a weight percentage range from 0.001 wt % to 99 wt %, 0.001 wt % to 75 wt %, 0.001 wt % to 50 wt%, 0.001 wt % to 25 wt%, 0.001 wt % to 10 wt %, 0.001 wt % to 5 wt %, 0.001 wt % to 2 wt %, 0.001 wt % to 1 wt %, 0.001 wt % to 0.1 wt %, 0.001 wt % to 0.01 wt %, 0.01 wt % to 99 wt %, 0.01 wt % to 75 wt %, 0.01 wt % to 50 wt%, 0.01 wt % to 25 wt%., 0.01 wt % to 10 wt %, 0.01 wt % to 5 wt %, 0.01 wt % to 2 wt %, 0.01 wt % to 1 wt %, 0.1 wt % to 99 wt %, 0.1 wt % to 75 wt %, 0.1 wt % to 50 wt%, 0.1 wt % to 25 wt%, 0.1 wt % to 10 wt %, 0.1 wt % to 5 wt %, 0.1 wt % to 2 wt %, 0.1 wt % to 1 wt %, 0.1 wt % to 0.5 wt %, 1 wt % to 99 wt %, 1 wt % to 75 wt %, 1 wt % to 50 wt%, 1 wt % to 25 wt%, 1 wt % to 10 wt %, 1 wt % to 5 wt %, 5 wt % to 99 wt %, 5 wt % to 75 wt %, 5 wt % to 50 wt%, 5 wt % to 25 wt%, 5 wt % to 10 wt %, 10 wt % to 99 wt %, 10 wt % to 75 wt %, 10 wt % to 50 wt%, 10 wt % to 25 wt%, 10 wt % to 15 wt %, 20 wt % to 99 wt %, 20 wt % to 75 wt %, 20 wt % to 50 wt%, 30 wt % to 99 wt %, 30 wt % to 75 wt %, 30 wt % to 50 wt%, 40 wt % to 99 wt %, 40 wt % to 75 wt %, 40 wt % to 50 wt%, 50 wt % to 99 wt %, 50 wt % to 75 wt %, 60 wt % to 99 wt %, 60 wt % to 75 wt %, 70 wt % to 99 wt %, 70 wt % to 75 wt %, 80 wt % to 99 wt %, 80 wt % to 90 wt %, or 90 wt % to 99 wt% of the tabletop sweetener composition.

### H. Medicinal Compositions

In some embodiments, the consumable product comprising one or more NHIS, one or more derivatives of NHIS, and/or one or more cooling agents is a medicinal composition. As used herein, the term “medicinal composition” includes solids, gases and liquids which are ingestible materials having medicinal value, such as cough syrups, cough drops, medicinal sprays, vitamins, and chewable medicinal tablets that are administered orally or used in the oral cavity in the form of e.g., a pill, tablet, spray, capsule, syrup, drop, troche agent, powder, and the like.

### I. Oral Hygiene Compositions

In some embodiments, the consumable product comprising one or more NHIS, one or more derivatives of NHIS, and/or one or more cooling agents is an oral hygiene composition. As used herein, the “oral hygiene composition” includes mouthwashes, mouth rinses, breath fresheners, toothpastes, tooth polishes, dentifrices, mouth sprays, teeth whitening agents, soaps, perfumes, and the like.

### J. Cosmetic Compositions

In some embodiments, the consumable product comprising one or more NHIS, one or more derivatives of NHIS, and/or one or more cooling agents is a cosmetic or skin-care product. As used herein, the term “cosmetic composition” means a composition that is formulated for topical application to skin, which has a pleasant color, odor and feel, and which does not cause unacceptable discomfort (stinging, tautness or redness) liable to discourage the consumer from using it.

Cosmetic composition may be preferably formulated in the form of an emulsion, e.g., W/O (water-in-oil), O/W (oil-in-water), W/O/W (water-in-oil-in-water), O/W/O (oil-in-water-in-oil) emulsion, PIT emulsion, Pickering emulsion, emulsion with a low oil content, micro- or nanoemulsion, a solution, e.g., in oil (fatty oils or fatty acid esters, in particular C6-C₃₂ fatty acid C₂-C₃₀ esters) or silicone oil, dispersion, suspension, creme, lotion or milk, depending on the production method and ingredients, a gel (including hydrogel, hydrodispersion gel, oleogel), spray (e.g., pump spray or spray with propellant) or a foam or an impregnating solution for cosmetic wipes, a detergent, e.g., soap, synthetic detergent, liquid washing, shower and bath preparation, bath product (capsule, oil, tablet, salt, bath salt, soap, etc.), effervescent preparation, a skin care product such as e.g., an emulsion (as described above), ointment, paste, gel (as described above), oil, balsam, serum, powder (e.g., face powder, body powder), a mask, a pencil, stick, roll-on, pump, aerosol (foaming, non-foaming or post-foaming), a deodorant and/or antiperspirant, mouthwash and mouth rinse, a foot care product (including keratolytic, deodorant), an insect repellent, a sunscreen, aftersun preparation, a shaving product, aftershave balm, pre- and aftershave lotion, a depilatory agent, a hair care product such as e.g., shampoo (including 2-in-1 shampoo, anti-dandruff shampoo, baby shampoo, shampoo for dry scalps, concentrated shampoo), conditioner, hair tonic, hair water, hair rinse, styling creme, pomade, perm and setting lotion, hair spray, styling aid (e.g., gel or wax), hair smoothing agent (detangling agent, relaxer), hair dye such as e.g., temporary direct-dyeing hair dye, semi-permanent hair dye, permanent hair dye, hair conditioner, hair mousse, eye care product, make-up, make-up remover or baby product.

### K. Smokable Compositions

In some embodiments, the consumable product comprising one or more NHIS, one or more derivatives of NHIS, and/or one or more cooling agents is a smokable composition. The term “smokable composition,” as used herein, includes any material that can be smoked or inhaled, such as tobacco and cannabis, as well as any smokable material that is burned to provide desirable aromas (e.g., charcoal briquettes for grilling foods, incense etc.). The smoking compositions may encompass cigarettes, electronic cigarettes (e-cigarettes), cigars, pipe and cigar tobacco, chew tobacco, vaporizable liquids, and all forms of tobacco such as shredded filler, leaf, stem, stalk, homogenized leaf cured, reconstituted binders, reconstituted tobacco from tobacco dust, fines, or other sources in sheet, pellet or other forms. “Smokable compositions” also include cannabis compositions (e.g., flower materials, leaf materials, extracts, oils, edible candies, vaporizable liquids, cannabis-infused beverages, etc.) and tobacco substitutes formulated from non-tobacco materials.

### IV. Taste Profiles and Taste Testing

Components of the compositions of the present application and methods regarding the same as described herein are useful for improved taste and aroma profiles of many consumable products relative to control samples. The phrase “taste profile”, which is interchangeable with “sensory profile” and “sweetness profile”, may be defined as the temporal profile of all basic tastes of a sweetener. The “temporal profile” may be considered to represent the intensity of sweetness perceived over time in tasting of the composition by a human, especially a trained “taster”. Carbohydrate and polyol sweeteners typically exhibit a quick onset followed by a rapid decrease in sweetness, which disappears relatively quickly on swallowing a food or beverage containing the same. In contrast, high intensity natural sweeteners typically have a slower sweet taste onset reaching a maximal response more slowly, followed by a decline in intensity more slowly than with carbohydrate and polyol sweeteners. This decline in sweetness is often referred to as “sweetness linger” and is a major limitation associated with the use of high intensity natural sweeteners.

In the context of taste tasting, the terms “improve”, “improved” and “improvement” are used interchangeably with reference to a perceived advantageous change in a composition or consumable product upon introduction of the compositions of the present application relative to the original taste profile of the composition or consumable product without the added compositions of the present application, such as improved coolness, improved, freshness, less bitterness, better sweetness, better sour taste, better aroma, better mouth feel, better flavor, less aftertaste, etc. The terms “improve” or “improvement” can refer to a slight change, a change, or a significant change of the original taste profile, etc., which makes the composition more palatable to an individual.

In some embodiments, the composition may be evaluated with reference to the degree of their sucrose equivalence. Accordingly, the components in the compositions of the present application may be diluted or modified with respect to its ingredients to conform with a desired sucrose equivalence.

The onset and decay of sweetness when the composition of the present application containing one or more NHIS, one or more derivatives of NHIS, and/or one or more cooling agents is consumed, the composition can be perceived by trained human tasters and measured in seconds from first contact with a taster’s tongue (“onset”) to a cutoff point (typically 180 seconds after onset) to provide a “temporal profile of sweetness”. A plurality of such human tasters is called a “sensory panel.” In addition to sweetness, sensory panels can also judge the temporal profile of the other “basic tastes”: bitterness, saltiness, sourness, piquance (aka spiciness), and umami (aka savoriness or meatiness). The onset and decay of bitterness when a sweetener is consumed, as perceived by trained human tasters and measured in seconds from first perceived taste to the last perceived aftertaste at the cutoff point, is called the “temporal profile of bitterness.” Aromas from aroma producing substances are volatile compounds which are perceived by the aroma receptor sites of the smell organ, i.e., the olfactory tissue of the nasal cavity. They reach the receptors when drawn in through the nose (orthonasal detection) and via the throat after being released by chewing (retronasal detection). The concept of aroma substances, like the concept of taste substances, is to be used loosely, since a compound might contribute to the typical aroma or taste of one food, while in another food it may cause a faulty aroma or taste, or both, resulting in an off-flavor. Thus, sensory profile may include evaluation of aroma as well.

The term “mouth feel” involves the physical and chemical interaction of a consumable in the mouth. More specifically, as used herein, the term “mouth feel” refers to the fullness sensation experienced in the mouth, which relates to the body and texture of the consumable such as its viscosity. Mouth feel is one of the most important organoleptic properties and the major criteria that consumers use to judge the quality and freshness of foods. Subtle changes in a food and beverage product’s formulation can change mouth feel significantly. Simply taking out sugar and adding a high intensity sweetener can cause noticeable alterations in mouth feel, making a formerly good product unacceptable to consumers. Sugar not only sweetens, it also builds body and viscosity in food and beverage products, and leaves a slight coating on the tongue. For example, reducing salt levels in soup changes not only taste, but can alter mouth feel as well. Primarily it is the mouth feel that is always the compliant with non-sugar sweeteners.

The phrase “sweetness detection threshold” refers to the minimum concentration at which panelists consisting of 1-10 persons are able to detect sweetness in a composition, liquid or solid. This is further defined as provided in the Examples herein and are conducted by the methods described in Sensory Testing for Flavorings with Modifying Properties by Christie L. Harman, John B. Hallagan, and the FEMA Science, Committee Sensory Data Task Force, November 2013, Volume 67, No. 11 and Appendix A attached thereto, the teachings of which are incorporated herein by reference.

“Threshold of sweetness” refers to a concentration of a material below which sweetness cannot be detected, but can still impart a flavor to a consumable (including water). When half of a trained panel of testers determines something is “sweet” at a given concentration, then the sample meets the threshold. When less than half of a panel of testers cannot discern sweetness at a given concentration, then concentrations of the substance below the sweetness level are considered a flavoring agent.

Production of the he composition of the present application may involve the use of any of the following methodologies, including reflux at atmospheric pressure, reaction under pressure, oven drying, vacuum oven drying, roller/drum drying, surface scraped heat exchange, and/or extrusion.

The inventors of the present application have also developed a unique process which can preserve useful flavor substances originating from natural high intensity sweetener plants, including stevia, sweet tea, monk fruit, licorice etc. and recovered in in the form of stevia extracts, sweet tea extracts, monk fruit extracts, licorice etc. Such flavor substances can be further amplified in glycosylation and/or Maillard reactions involving the foregoing extracts in combination with various amine donors as described herein.

Additionally, flavor substances in natural high intensity sweetener plants can also include new flavor substances from new natural high intensity sweetener plant varieties produced by hybridizing, grafting and other cultivating methods.

A flavoring agent, other than a flavor derived from a Maillard reaction product as described herein, can be added to the compositions described herein before or after a Maillard reaction has been effected. Suitable flavoring agents include, for example, natural flavors, vitamins, such as vitamin C, artificial flavors, spices, seasonings, and the like. Exemplary flavor agents include synthetic flavor oils and flavoring aromatics and/or oils, uronic acids (e.g., glucuronic acid and galacturonic acid) or oleoresins, essences, and distillates, and a combination comprising at least one of the foregoing.

During the Maillard reaction or following completion of the Maillard reaction, “top note” agents may be added, which are often quite volatile, vaporizing at or below room temperature. “Top notes” are often what give foods their fresh flavors. Suitable top note agents include but are not limited to, for example, furfuryl mercaptan, methional, nonanal, trans,trans-2,4-decadienal, 2,2′-(dithiodimethylene) difuran, 2-methyl-3-furanthiol, 4-methyl-5-thiazoleethanol, pyrazineethanethiol, bis(2-methyl-3-furyl) disulfide, methyl furfuryl disulfide, 2,5-dimethyl-2,5-dihydroxy-1,4-dithiane, 95%, trithioacetone, 2,3-butanedithiol, methyl 2-methyl-3-furyl disulfide, 4-methylnonanoic acid, 4-methyloctanoic acid, or 2-methyl-3-tetrahydrofuranthiol.

Flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents include artificial, natural and synthetic fruit flavors, such as vanilla, and citrus oils including lemon, orange, lime, grapefruit, yuzu, sudachi, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth.

Additional exemplary flavors imparted by a flavoring agent include a milk flavor, a butter flavor, a cheese flavor, a cream flavor, and a yogurt flavor; a vanilla flavor; tea or coffee flavors, such as a green tea flavor, an oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, a wasabi (Japanese horseradish) flavor; a nut flavor, such as an almond flavor, a hazelnut flavor, a macadamia nut flavor, a peanut flavor, a pecan flavor, a pistachio flavor, and a walnut flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor.

Generally any flavoring agent or food additive, such as those described in “Chemicals Used in Food Processing”, Publication No 1274, pages 63-258, by the National Academy of Sciences, can be used. This publication is incorporated herein by reference.

As used herein, a “flavoring agent” or “flavorant” herein refers to a compound or an ingestibly acceptable salt or solvate thereof that induces a flavor or taste in an animal or a human. The flavoring agent can be natural, semi-synthetic, or synthetic. Suitable flavorants and flavoring agent additives for use in the compositions of the present application include, but are not limited to, vanillin, vanilla extract, mango extract, cinnamon, citrus, coconut, ginger, viridiflorol, almond, bay, thyme, cedar leaf, nutmeg, allspice, sage, mace, menthol (including menthol without mint), an essential oil, such as an oil produced from a plant or a fruit, such as peppermint oil, spearmint oil, other mint oils, clove oil, cinnamon oil, oil of wintergreen, or an oil of almonds; a plant extract, fruit extract or fruit essence from grape skin extract, grape seed extract, apple, banana, watermelon, pear, peach, grape, strawberry, raspberry, cherry, plum, pineapple, apricot, a flavoring agent comprising a citrus flavor, such as an extract, essence, or oil of lemon, lime, orange, tangerine, grapefruit, citron, kumquat, or combinations thereof. Flavorants for use in the present application include both natural and synthetic substances which are safe for humans or animals when used in a generally accepted range.

Non-limiting examples of proprietary flavorants include Dohler™ Natural Flavoring Sweetness Enhancer K14323 (Dohler™, Darmstadt, Germany), Symrise™ Natural Flavor Mask for Sweeteners 161453 and 164126 (Symrise™, Holzminden, Germany), Natural Advantage™ Bitterness Blockers 1, 2, 9 and 10 (Natural Advantage™, Freehold, New Jersey, U.S.A.), and Sucramask™ (Creative Research Management, Stockton, California, U.S.A.).

In the any of the embodiments described in the present application, the flavoring agent may be present in the composition of the present application in an amount effective to provide a final concentration of about 0.1 ppm, 0.5 ppm, 1 ppm, 2 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 100 ppm, 110 ppm, 120 ppm, 130 ppm, 140 ppm, 150 ppm, 160 ppm, 170 ppm, 180 ppm, 190 ppm, 200 ppm, 220 ppm, 240 ppm, 260 ppm, 280 ppm, 300 ppm, 320 ppm, 340 ppm, 360 ppm, 380 ppm, 400 ppm, 425 ppm, 450 ppm, 475 ppm, 500 ppm, 550 ppm, 600 ppm, 650 ppm, 700 ppm, 750 ppm, 800 ppm, 850 ppm, 900 ppm, 950 ppm, 1000 ppm, 1500 ppm, 2000 ppm, 2500 ppm, 3000 ppm, 3500 ppm, 4000 ppm, 4500 ppm, 5000 ppm, 6000 ppm, 7000 ppm, 8000 ppm, 9000 ppm, 10,000 ppm, 11,000 ppm, 12,000 ppm, 13,000 ppm, 14,000 ppm, or 15,000 ppm; or to provide a final concentration corresponding to any one of the aforementioned values in this paragraph; or to provide a final concentration range corresponding to any pair of the aforementioned values in this paragraph.

In more particular embodiments, the flavoring agent may be present in the composition of the present application in an amount effective to provide a final concentration ranging from 10 ppm to 1000 ppm, from 50 ppm to 900 ppm, from 50 ppm to 600 ppm, from 50 ppm to 500 ppm, from 50 ppm to 400 ppm, from 50 ppm to 300 ppm, from 50 ppm to 200 ppm, from 75 ppm to 600 ppm, from 75 ppm to 500 ppm, from 75 ppm to 400 ppm, from 75 ppm to 300 ppm, from 75 ppm to 200 ppm, from 75 ppm to 100 ppm, from 100 ppm to 600 ppm, from 100 ppm to 500 ppm, from 100 ppm to 400 ppm, from 100 ppm to 300 ppm, from 100 ppm to 200 ppm, from 125 ppm to 600 ppm, from 125 ppm to 500 ppm, from 125 ppm to 400 ppm, from 125 ppm to 300 ppm, from 125 ppm to 200 ppm, from 150 ppm to 600 ppm, from 150 ppm to 500 ppm, from 150 ppm to 500 ppm, from 150 ppm to 400 ppm, from 150 ppm to 300 ppm, from 150 ppm to 200 ppm, from 200 ppm to 600 ppm, from 200 ppm to 500 ppm, from 200 ppm to 400 ppm, from 200 ppm to 300 ppm, from 300 ppm to 600 ppm, from 300 ppm to 500 ppm, from 300 ppm to 400 ppm, from 400 ppm to 600 ppm, from 500 ppm to 600 ppm; or to provide a final concentration corresponding to any one of the aforementioned values in this paragraph; or to provide a final concentration range corresponding to any pair of the aforementioned values in this paragraph.

### EXAMPLES

### Example 1. Preparation of Glycosylated Rubusoside 90% (GRU90) from Rubusoside 90%

Glycosylated reaction products from Rubusoside 90% were prepared according to the following method.

Rubusoside 90%, available from EPC Natural Products Co., Ltd. The content of RU is 92.8%. Lot# 238-34-03

i) 15 g dextrin was dissolved in 45 ml deionized water.

ii) 15 g Rubusoside 90% was added to liquefied dextrin.

iii) 0.75 ml CGTase enzyme and 15ml deionized water were added to the mixture of ii) and incubated at 69° C. for 20 hours to glycosylate the RU90 composition via glucose molecules derived from tapioca dextrin.

iv) The reaction mixture was heated to 85° C. for 10 min to inactivate the CGTase, which was then removed by filtration.

v) The resulting solutions of GRU90, residual RU and dextrin were decolored and spray dried yielding 25 g of GRU90 (product 1 of Example 1) as a white powder (the content of residue RU is 12.16%).

### Example 2. Preparation of GRU90-MRP-FTAs From GRU90, Fructose and Glutamic Acid

GRU90: the product of Example 1.

GRU90, fructose, glutamic acid and water were weighed and dissolved in water according to Table 2-1. The solutions were then heated at about 100° C. for 1.5 hour. When the reactions were completed, the solutions were filtered through filter paper and the filtrates were dried with a spray dryer, thereby resulting in 2-01 and 2-02 products as an off white powder.

TABLE 2-1

Sample compositions.

| Product No. | Weight of GRU90 (g) | Weight of fructose (g) | Weight of glutamic acid (g) | Weight of water (g) |
|---|---|---|---|---|
| 2-01 | 9 | 0.5 | 0.5 | 5 |
| 2-02 | 6 | 3.696 | 0.308 | 5 |

### Example 3. Preparation of GSG-MRP-PLTA and GRU90-MRP-PLTA from GRU90, GSGs, Fructose, Glutamic Acid and Piper Longum Extract

Raw materials:

GRU90: the product of Example 1.

GSGs (glycosylated stevia extract comprises unreacted steviol glycosides), available from Sweet Green Fields. Lot#: 3080191. Preparing procedure was similar as in Example 1, except RU90 was replaced with Stevia extract. The content of residual dextrin is 14.3%, total steviol glycosides is 85.7%, including unreacted and glycosylated steviol glycosides, among them Rebaudioside A is 9.11% and stevioside is 4.45%

Piper longum extract: 100 g Piper longum was broken to 0.3-0.5 cm small pieces and mixed with 250 ml ethanol. The mixture was then extract at 45° C. for 6 h by Soxhlet extractor. When the extract was completed, the solution was concentrated to a paste.

Process: GRU90, GSGs, fructose, glutamic acid, Piper longum extract, water were weighed as follows. The solution was then heated at about 95-100° C. for 1.5 hour. When the reaction was completed, the solution was filtered through filter paper and the filtrate was dried with a spray dryer, thereby resulting in products 3-01 to 3-02 as an off white powder.

TABLE 3-1

Test sample compositions.

| Product name | Product No. | Weight of GSGs (g) | Weight of GRU90 (g) | Weight of fructose (g) | Weight of glutamic acid (g) | Weight of water (mL) | Weight of Piper longum extract (g) |
|---|---|---|---|---|---|---|---|
| GRU90-MRP-PLTA | 3-01 | 20 | | 1.5 | 0.5 | 11 | 0.4 |
| GSG-MRP-PLTA | 3-02 | | 20 | | | | |

### Example 4. RU90, GRU90 (Product 1 in Example 1), GRU90-MRP-FTA (Product 2-01 and 2-02 in Example 2), GRU90-MRP-PLTA (Product 3-01 in Example 3) and GSG-MRP-PLTA (Product 3-02 in Example 3) Improve the Taste Profile of Mouthwash

Listerine cool mint mouthwash: available from Johnson & Johnson China Co., LTD. Lot#:6114743.

Ingredients: water, ethanol, sorbitol, thymol, eucalyptol, methyl salicylate, menthol, sodium benzoate, benzoic acid, poloxamer 407, saccharin sodium salt, C142053, edible essence.

Process: Dissolve a certain amount of concentrated RU90, GRU90 (product 1 in Example 1), GRU90-MRP-FTA (products 2-01 and 2-02 in Example 2), GRU90-MRP-PLTA (product 3-01 in Example 3) and GSG-MRP-PLTA (product 3-02 in Example 3) into the mouthwash.

The details are as follows.

TABLE 4-1

Sample compositions

| Type of added products | Weight of added products (mg) | Volume of Listerine cool mint mouthwash (mL) | Concentration of added products (ppm) |
|---|---|---|---|
| Base | - | 100 | - |
| RU90 | 2 | 100 | 20 |
| GRU90 (product 1 in Example 1) | 5 | 100 | 50 |
| GRU90-MRP-FTA (product 2-01 in Example 2) | 5 | 100 | 50 |
| GRU90-MRP-FTA (product 2-02 in Example 2) | 3 | 100 | 30 |
| GRU90-MRP-PLTA (product 3-01 in Example 3) | 5 | 100 | 50 |
| GSG-MRP-PLTA (product 3-02 in Example 3) | 5 | 100 | 50 |

Sensory evaluation method:

1) Coolness

Menthol (available from ADM Food Technology Co., Ltd. Lot#:8808002077) concentration of 50-200 ppm was the coolness standard, and the specific coolness scoring standards are shown in Table 4-2.

TABLE 4-2

Coolness evaluation test standard.

| Range of menthol concentration | <50 ppm | 50-100 ppm | 100-150 ppm | 150-200 ppm | >200 ppm |
|---|---|---|---|---|---|
| Score of coolness level | 1 | 2 | 3 | 4 | 5 |

Evaluation method:

The sample to be evaluated was dissolved in neutral deionized water. The tester placed 20-30 mL of the evaluation solution in their mouth. After 5 seconds the solution was spit out. After a mouthwash step with water, the standard solution was taken. If the degree of coolness was similar, the coolness degree of the sample solution could be determined as the coolness degree value of the standard solution. Otherwise it was necessary to take additional standard solutions and try again until the coolness degree value was determined.

Experiment: Each sample was evaluated according to the aforementioned sensory evaluation method and the above added method. The average score of the panel was taken as the evaluation result data. The taste profile of the mixture is shown in Table 4-3.

TABLE 4-3

Sensory evaluation results.

| Sample | Overall likability | Coolness | Refreshing | Bitterness | Sweet lingering |
|---|---|---|---|---|---|
| Base | 2.5 | 2.5 | 3 | 4 | 4 |
| RU90 | 3 | 3 | 3.5 | 3 | 3 |
| GRU90 | 3.5 | 3.5 | 4 | 2.5 | 3 |
| GRU90-MRP-FTA (product 2-01 in Example 2) | 4 | 4 | 4.5 | 2 | 2.5 |
| GRU90-MRP-FTA (product 2-02 in Example 2) | 4.5 | 4.5 | 4 | 1.5 | 2 |
| GRU90-MRP-PLTA (product 3-01 in Example 3) | 3.5 | 3 | 3 | 2.5 | 3 |
| GSG-MRP-PLTA (product 3-02 in Example 3) | 3.5 | 3 | 3 | 2.5 | 3 |

Conclusion: RU90, GRU90 (product 1 in Example 1), GRU90-MRP-FTA (products 2-01 and 2-02 in Example 2), GRU90-MRP-PLTA (product 3-01 in Example 3) and GSG-MRP-PLTA (product 3-02 in Example 3) all can improved the taste profile of Listerine cool mint mouthwash. RU90 and other subsequent processing products can reduce the unpleasant bitterness and sweet lingering in Listerine cool mint mouthwash. Specifically, GRU90, GRU90-MRP-FTA (product 2-01 and 2-02 in Example 2) provided significant enhancements in coolness and refreshing and GRU90-MRP-PLTA (product 3-01 in Example 3) and GSG-MRP-PLTA (product 3-02 in Example 3) can provide significant enhancements in biting or sting. The result showed that RU90, GRU90, and its MRPs can improve the taste profile of mouthwash. This result can extend to all the stevia extract, glycosylated stevia extract and their MRPs.

### Example 5. Preparation of Glycosylated Steviol Glycosides, Such As Glycosylated Steviolbioside 90% (GSTB90) and Glycosylated Rebaudioside A 100% (GRA100)

A glycosylated steviol glycoside was prepared using steviol glycoside such as steviolbioside (STB90) or Rebaudioside A (RA100) as materials (the contents and source are shown in Table 5-1) according to the following method:

TABLE 5-1

Contents and source of STB90 and RA100 (detected by JECFA 2010 method)

| | Company | Lot#. | Contents |
|---|---|---|---|
| STB90 | EPC Natural Products Co., Ltd. | 316-58-01 | 92.12% |
| RA100 | Sweet Green Fields | CT001-140604 | 100.03% |

i) 15 g maltodextrin (BAOLINGBAO BIOLOGY Co., Ltd) was dissolved in 45 mL deionized water

ii) 15 g steviol glycoside was added to the dissolved dextrin solution to form a mixture.

iii) 0.75 mL CGTase enzyme (Amano Enzyme, Inc.) and 15 mL deionized water were added to the mixture and incubated at 69° C. for 20 hours to glycosylate the steviol glycoside with glucose molecules derived from maltodextrin.

iv) The reaction mixture of iii) was heated to 85° C. for 10 min to inactivate the CGTase, which was then removed by filtration.

v) The resulting solution of glycosylated steviol glycoside such as GSTB or GRA, residual steviol glycoside such as STB or RA and dextrin were decolored and spray dried, thereby yielding GSTB90 or GRA100 as white powder.

### Example 6. Preparation of Flavored GSTB90-MRP-FTA and GRA100-MRP-FTA from GSTB90 and GRA100, Glutamic Acid and Fructose

GSTB90 and GRA100: the products of Example 5.

Procedure: GSTB90 or GRA100, respectively, fructose, glutamic acid and water were weighed as shown in Table 6-1 and then mixed. The solution was then heated at about 100oC for 1.5 hour. When the reaction was completed, the solution was filtered through filter paper and the filtrate was dried with a spray dryer, thereby obtained off white powder named as 6-01 or 6-02, respectively.

TABLE 6-1

Sample compositions.

| Product No. | Weight and type of GSTB90 and GRA100 | Weight of fructose (g) | Weight of glutamic acid (g) | Weight of water (g) |
|---|---|---|---|---|
| 6-01 | GSTB90/9g | 0.5 g | 0.5 g | 5 g |
| 6-02 | GRA100/9g | | | |

### Example 7. STB90, GSTB90 and GSTB90-MRP-FTA (Product 6-01 in Example 6) RA100, GRA100 and GRA100-MRP-FTA (Product 6-02 in Example 6) Improved the Taste Profile of Mouthwash

Crest White lock mouthwash (peach flavor): available from Guangzhou P & G Co., Ltd. Lot#:1207D29811

Ingredients: water, glycerinum, propylene glycol, sodium hexametaphosphate , ethanol, sorbitol, thymol, eucalyptol, methyl salicylate, menthol, benzoic acid, poloxamer 407, sodium benzoate, Sodium lauryl sulfate, edible essence, phosphoric acid, saccharin sodium salt, Trichlorogalactose, Cl 17200, Cl19140.

Process: Dissolve a certain amount of concentrated STB90, GSTB90 and GSTB90-MRP-FTA (product 6-01 in Example 6) RA100, GRA100 and GRA100-MRP-FTA (product 6-02 in Example 6) the mouthwash.

The details are as follows.

TABLE 7-1

Sample compositions.

| Type of added products | Weight of added products (mg) | Volume of mouthwash (mL) | Concentration (ppm) |
|---|---|---|---|
| Base | - | 100 | - |
| STB90 | 5 | 100 | 50 |
| GSTB90 | 5 | 100 | 50 |
| GSTB90-MRP-FTA (product 6-01 in Example 6) | 5 | 100 | 50 |
| RA100 | 5 | 100 | 50 |
| GRA100 | 5 | 100 | 50 |
| GRA100-MRP-FTA (product 6-02 in Example 6) | 5 | 100 | 50 |

Experiment: Each sample was evaluated according to the aforementioned sensory evaluation method and the above added method. The average score of the panel was taken as the evaluation result data. The taste profile of the mixture is shown in Table 7-2.

TABLE 7-2

Sensory evaluation results

| | Overall likability | Coolness | Refreshing | Bitterness | Flavor |
|---|---|---|---|---|---|
| Base | 2.5 | 2.5 | 3 | 3 | 3 |
| STB90 | 3 | 3 | 3.5 | 3 | 3 |
| GSTB90 | 4 | 4 | 4 | 1.5 | 4 |
| GSTB90-MRP-FTA (product 6-01 in Example 6) | 4.5 | 4 | 4.2 | 1 | 4 |
| RA100 | 3.5 | 3.2 | 3.5 | 2 | 3.5 |
| GRA100 | 3.8 | 3.5 | 4 | 1.5 | 3.5 |
| GRA100-MRP-FTA (product 6-02 in Example 6) | 4 | 3.5 | 4.5 | 1 | 4 |

Conclusion: STB90, GSTB90 and GSTB90-MRP-FTA (product 6-01 in Example 6), RA100, GRA100 and GRA100-MRP-FTA (product 6-02 in Example 6) all can improved the taste profile of Crest White lock mouthwash. Specifically, GSTB90 and other subsequent processing products can reduce the unpleasant bitterness, improve peach flavor and coolness in mouthwash.

### Example 8. GRU90-MRP-FTA (Product 2-02 in Example 2) Improved The Coolness of Essential Embrocations

Essential Embrocation 1, (liquid state), available from Shanghai Zhonghua Pharmaceutical Nantong Co., Ltd. Lot#:C200813

Ingredients: menthol, methyl salicylate, camphor, eucalyptus oil, eugenol, essence, chlorophyll, liquid paraffin

Essential Embrocation 2, (Cooling nasal relief inhalant) available from Shanghai Zhonghua Pharmaceutical Co., Ltd. Lot#:C210201

Ingredients: menthol, Peppermint oil, methyl salicylate, camphor, eucalyptus oil, palchouli oil, lavender oil

Essential Embrocation 3, (solid state) available from Shanghai Zhonghua Pharmaceutical Co., Ltd. Lot#:200507

Ingredients: menthol, Peppermint oil, camphor, eucalyptus oil, clove oil, cinnamon oil, ammonia, water.

Process: Dissolve a certain amount of GRU90-MRP-FTA (product 2-02 in Example 2) into the Essential Embrocation. The details are as follows.

TABLE 8-1

Sample compositions.

| Sample | Weight of GRU90-MRP-FTA (product 2-02 in Example 2) | Volume/weight of Essential Embrocation | Evaluation type |
|---|---|---|---|
| Essential Embrocation 1 | - | 100 mL | temple smear |
| Essential Embrocation 1+ GRU90-MRP-FTA (product 2-02 in Example 2) | 10 mg | 100 mL | |
| Essential Embrocation 2 | - | 0.6 g | nose inhalation |
| Essential Embrocation 2+ GRU90-MRP-FTA (product 2-02 in Example 2) | 0.01 g | 0.6 g | |
| Essential Embrocation 3 | - | 0.1 g | temple smear |
| Essential Embrocation 3+ GRU90-MRP-FTA (product 2-02 in Example 2) | 0.01 | 0.1 g | |

Experiment: Each sample was evaluated by coolness feeling of temple smear or nose inhalation. The sensory evaluation results of the mixture are shown in Table 8-2.

TABLE 8-2

Sensory evaluation results.

| | | |
|---|---|---|
| Sample | Evaluation type | Sensory evaluation |
| Essential Embrocation 1 | temple smear | The coolness onset of Essential Embrocation 1 is 5 s while GRU90-MRP-FTA (product 2-02 in Example 2) makes it earlier to 2 s. What is more, GRU90-MRP-FTA (product 2-02 in Example 2) makes coolness intensity of Essential Embrocation 1 higher at the beginning and equal to each other at 6 min. The coolness lasting of adding GRU90-MRP-FTA (product 2-02 in Example 2) is 120 min while Essential Embrocation 1 itself is 110 min. |
| Essential Embrocation 1+ GRU90-MRP-FTA (product 2-02 in Example 2) | | |
| Essential Embrocation 2 | nose inhalation | Adding GRU90-MRP-FTA (product 2-02 in Example 2) makes the coolness intensity higher and its sensation spread to the throat compared to the no adding. Specially, GRU90-MRP-FTA (product 2-02 in Example 2) makes the flavor more balance. |
| Essential Embrocation 2+GRU90-MRP-FTA (product 2-02 in Example 2) | | |
| Essential Embrocation 3 | temple smear | The coolness onset of Essential Embrocation 1 is 3 min while GRU90-MRP-FTA (product 2-02 in Example 2) makes it earlier to 1 min. What is more, GRU90-MRP-FTA (product 2-02 in Example 2) makes the coolness intensity of Essential Embrocation 1 higher in the whole feeling period. The coolness lasting of adding GRU90-MRP-FTA (product 2-02 in Example 2) is 2h while Essential Embrocation 3 is 1.5 h. |
| Essential Embrocation 3+ GRU90-MRP-FTA (product 2-02 in Example 2) | | |

Conclusion: GRU90-MRP-FTA (product 2-02 in Example 2) can significantly improve coolness onset, intensity and lasting of Essential Embrocation no mater by temple smear or nose inhalation. What is more, GRU90-MRP-FTA (product 2-02 in Example 2) can balance the flavor of flavored Essential Embrocation and makes its sensation spread to the throat. The results show GRU90-MRP-FTA (product 2-02 in Example 2) can improve the coolness of Essential Embrocation. This result can extend to all stevia extract, glycosylated stevia extract and their MRPs.

### Example 9. GRU90-MRP-FTA (Product 2-02 in Example 2) Improved The Taste Profile of an Essential Embrocation

Essential Embrocation, available from Shanghai Zhonghua Pharmaceutical Nantong Co., Ltd. Lot#: C200813

Ingredients: menthol, methyl salicylate, camphor, eucalyptus oil, eugenol, essence, chlorophyll, liquid paraffin

Process: Dissolve a certain amount of GRU90-MRP-FTA (product 2-02 in Example 2) into the diluted Essential Embrocation.

The details are as follows.

TABLE 9-1

Sample compositions.

| Prod uct No. | Weight of GRU90-MRP-FTA (product 2-02 in Example 2) (mg) | Weight of Essential Embrocati on (mg) | Volume of water (mL) | Concentration of GRU90-MRP-FTA (product 2-02 in Example 2) (ppm) | Concentratio n of Essential Embrocation (ppm) |
|---|---|---|---|---|---|
| Base | | 10 | 100 | | 100 |
| 2-02 | 5 | 10 | 100 | 50 | 100 |

Experiment: Each sample was evaluated according to the aforementioned sensory evaluation method and the above added method. The average scores from the panel were taken as the evaluation test results. The taste profile of the mixture is shown in Table 9-2.

TABLE 9-2

Sensory evaluation results.

| Product No. | Overall likability | Coolness | Refreshing | Bitterness |
|---|---|---|---|---|
| Base | 2.5 | 2.5 | 3 | 3 |
| 2-02 | 4 | 4 | 3.5 | 1.5 |

Conclusion: GRA100-MRP-FTA (product 6-02 in Example 6) can significantly reduce bitterness, enhance coolness, refreshing of Essential Embrocation, resulting in a better overall likability. The results showed that the taste profile of Essential Embrocation can be improved by GRA100-MRP-FTA (product 6-02 in Example 6). This result can extend to all stevia extract, glycosylated stevia extract and their MRPs.

### Example 10. Preparation of Glycosylated Rubusoside 90% (GRU90) from Rubusoside 90%

Glycosylated reaction products from Rubusoside 90% were prepared according to the following method.

Raw material:

Rubusoside 90% (available from EPC Natural Products Co., Ltd. The content of RU is 92.8% Lot# EPC-238-34-03)

i) 10 g tapioca dextrin was dissolved in 45 ml deionized water.

ii) 14.5 g Rubusoside 90% was added to liquefied dextrin.

iii) 0.5 ml CGTase enzyme and 15ml deionized water were added to the mixture of ii) and incubated at 69° C. for 20 hours to glycosylate the RU90 composition via glucose molecules derived from tapioca dextrin.

iv) The reaction mixture was heated to 85° C. for 10 min to inactivate the CGTase, which was then removed by filtration.

v) The resulting solution of GRU90, residual RU and dextrin were decolored and spray dried yielding 20 g of GRU90 as a white powder (the content of residue RU is 7.26%).

### Example 11. Preparation of GRU90-MRP-FTAs From GRU90, Fructose/ Concentrated Apple Juice and Glutamic Acid

Raw material:

GRU90: the product of Example 10.

Decolorized and deacidified concentrated apple juice (fructose content: 36.77%), available from China Haisheng Fresh Fruit Juice Co., Ltd, Weinan Branch, lot #: 25191005B01-05; 2)

Process: GRU90, fructose/concentrated apple juice, glutamic acid, water were weighed as shown in Table 11-1. All the ingredients were mixed and fully dissolved in the water. The solution was then heated at about 100° C. for 1.5 hours. When the reaction was completed, the solution was filtered through filter paper and the filtrate was dried with a spray dryer, resulting in products 11-01 to 11-02 as off-white powders.

TABLE 11-1

| Product No. | Weight of GRU90 (g) | Weight of glutamic acid (g) | Weight of fructose (g) | Weight of apple juice (g) | Weight of water (mL) | Reaction time |
|---|---|---|---|---|---|---|
| 11-01 | 6 | 0.307 | 3.692 | - | 5 | 1.5 |
| 11-02 | 18 | 1 | - | 15.82 | 4 | 1.5 |

### Example 12 Preparation of GSG-MRP-CA From GSG, Xylose and Alanine

Raw material:

GSGs (glycosylated stevia extract comprises unreacted steviol glycosides), available from Sweet Green Fields. Lot#: 3080191. Preparation procedure was similar as in Example 10, except RU90 was replaced with Stevia extract.

Process: GSGs, xylose and alanine, water were weighed as shown in Table 12-1. All the ingredients were mixed and fully dissolved in water. The solution was then heated at about 100° C. for 2 hours. When the reaction was completed, the solution was filtered through filter paper and the filtrate was dried with a spray dryer, resulting in product 12-01 as an off-white powder.

TABLE 12-1

| Product No. | Weight of GSG (g) | Weight of alanine (g) | Weight of xylose (g) | Weight of water (mL) | Reaction time |
|---|---|---|---|---|---|
| 12-01 | 8 | 0.5 | 1.5 | 60 | 2 |

### Example 13 Conversion of Rubusoside From Steviol Glycosides

Material: Steviol glycosides, available from Sweet Green Fields, Lot#: STV95-YCJ20200618. The contents of the steviol glycosides are shown in Table 13-1.

TABLE 13-1

Contents of steviol glycosides (m/m %)

| Lot# | RD | RA | STV | RF | RC | Dulc A | RU | RB | STB | TSG(9) |
|---|---|---|---|---|---|---|---|---|---|---|
| STV95-YCJ20200 618 | 0.02 | 2.13 | 95.7 | 0.01 | 0.17 | 0.07 | 0.33 | 0.01 | 0.36 | 98.82 |

Note: TSG refers to the content of total Steviol glycosides (TSG(9)), which includes Rebaudioside A, Rebaudioside B, Rebaudioside C, Rebaudioside D, Rebaudioside F, stevioside, steviolbioside, rubusoside, and dulcoside A.

Process:

1L of the steviol glycoside solution (100 g/L) and 1.5 g 0-galactosidase (0.8 kU/g stevioside) were mixed, and stirred at 60° C. for 12 h. The reaction mixture was then boiled for 3 min to deactivate the enzyme and the precipitated enzyme was removed by centrifugation. The filtrate was separated and purified by passing through an 800 mL T-28 macroporous resin column with 1600 mL water. The column was then washed with 1600 mL ethanol, and the solution was collected, decompressed and concentrated. The ethanol was later removed and the solution was spray-dried, resulting in RU products. The supernatant was spray-dried. The steviol glycoside contents of the powder obtained are described in Table 13-2.

TABLE 13-2

Contents of steviol glycosides (m/m %)

| Produ ct No. | Sample | RD | RA | STV | RF | RC | Dulc A | RU | RB | STB | TSG(9) |
|---|---|---|---|---|---|---|---|---|---|---|---|
| 13-01 | Rubusoside from steviol glycosides | / | 1.74 | 0.58 | / | / | / | 90.05 | / | / | 92.37 |

Conclusion: Stevioside can be converted to rubusoside by β-galactosidase. Under certain conditions, the conversion rate can be close to 100%.

### Example 14. Preparation of Glycosylated Rubusoside Derived From Steviol Glycoside Conversion

A glycosylated reaction product composition was prepared by steviol glycoside conversion according to the following method:

i) 15 g maltodextrin (BAOLINGBAO BIOLOGY Co., Ltd) was dissolved in 45 mL deionized water

ii) 15 g rubusoside derived from steviol glycosides conversion (product 13-01 in Example 13) was added to the dissolved dextrin solution to form a mixture.

iii) 0.75 mL CGTase enzyme (Amano Enzyme, Inc.) and 15 mL deionized water were added to the mixture and incubated at 69° C. for 20 hours to glycosylate the rubusoside from steviol glycoside conversion with glucose molecules derived from maltodextrin.

iv) The reaction mixture of iii) was heated to 85° C. for 10 min to inactivate the CGTase, which was then removed by filtration.

v) The resulting solution of glycosylated rubusoside (GRU), residual RU and dextrin were decolored and spray dried, thereby yielding 25 g glycosylated rubusoside product derived from steviol glycosides (GRUds) and forming a white powder therefrom.

### Example 15. Preparation of GRUds-MRP-Ftas From Gruds, Fructose/ Concentrated Apple Juice and Glutamic Acid

Raw materials:

GRUds: the product of Example 14.

Decolorized and deacidified concentrated apple juice (fructose content: 36.77%), available from China Haisheng Fresh Fruit Juice Co., Ltd, Weinan Branch, lot #: 25191005B01-05; 2)

Process: GRUds, fructose/concentrated apple juice, glutamic acid, water were weighed as shown in Table 15-1. All the ingredients were mixed and fully dissolved in the water. The solution was then heated at about 100° C. for 1.5 hours. When the reaction was completed, the solution was filtered through filter paper and the filtrate was dried with a spray dryer, resulting in white powder products 15-01 to 15-02 as shown in Table 15-1.

TABLE 15-1

| Product No. | Weight of GRUds (g) | Weight of glutamic acid (g) | Weight of fructose (g) | Weight of apple juice (g) | Weight of water (mL) | Reaction time |
|---|---|---|---|---|---|---|
| 15-01 | 6 | 0.307 | 3.692 | - | 5 | 1.5 |
| 15-02 | 18 | 1 | - | 15.82 | 4 | 1.5 |

### Example 16. GRU90-MRP-FTA (Product 11-01 From Example 11) Improves the Taste Profile of Menthol Solutions

Raw Materials:
GRU90-MRP-FTA (product 11-01 from Example 11)
Menthol

Sample preparation: raw materials were weighed as shown in Table 16-1.

| Sample No. | Water [ml] | Menthol [ppm] | GRU90-MRP-FTA(product 11-01 from Example 11) [ppm] |
|---|---|---|---|
| 16-01 | 100 | 15 | - |
| 16-02 | 100 | 15 | 10 |

Experiment: Each sample was evaluated and average scores from the test panel for each sensory criterium were recorded as the evaluation test results. The resulting taste profiles of the mixtures are shown in Table 16-2.

TABLE 16-2

Sensory evaluations.

| | |
|---|---|
| 16-01 | Cooling: on the tongue and in the throat, slight bitter aftertaste from peppermint flavor |
| 16-02 | More intensive cooling effect, rounder and smoother peppermint flavor, no bitter aftertaste, area of cooling: on the tongue and then in the throat |

### Example 17. GRU-MRPs Improve the Taste Profile of Soft Candies With Peppermint Flavor

Raw Materials:
Gelatin, Z1243 Mat.-Nr. 2-01-420789/003, Dr. Oetker
Isomalt E953, Charge/Lot: 038907091112, Bombaser Decor AG
Crystalline Maltitol, Lot v# GNS909172
Food color green, 08.22L039 A03 09:42/21, Dr. Oetker
The mixture of GSG-MRP-CA (product 12-01 from Example 12) and GRUds-MRP-FTAs (product 15-02 from Example 15)
GRU90-MRP-FTA: product 11-01 from Example 11
GRU90-MRP-FTA: product 11-02 from Example 11

Sample preparation:

The samples were weighed as shown in Table 17-1 and subjected to the following steps:

i) Food color was dissolved in water (ca. 5 drops/100 ml)

ii) Gelatin was added to 50 ml of water/food color solution and soaked for ca. 10 min. The gelatin was dissolved in low heat with occasional stirring. The liquid should not be boiling hot.

iii) Isomalt and maltitol were added to the gelatin alone or in combination with: (a) a mixture of GSG-MRP-CA (product 12-01 from Example 12) and GRUds-MRP-FTAs (product 15-02 from Example 15; (b) GRU90-MRP-FTA (product 11-01 from Example 11); or (c) GRU90-MRP-FTA (product 11-02 from Example 11) and dissolved with stirring in the gelatin solution. The liquid should not be boiling hot.

iv) The candy mass was removed from the heat and 20 µl of peppermint oil was added to the samples and mixed thoroughly.

v) The candy mass was filled into a silicon form and cooled in the refrigerator.

TABLE 17-1

Sample formulations

| Sample | Water [ml] | Gelatin [g] | Isomalt [g] | Maltitol [mg] | Mixture of GSG-MRP-CA (product 12-01 from Example 12) and GRUds-MRP-FTAs (product 15-02 from Example 15) [mg] | GRU90-MRP-FTA (product 11-02 from Example 11) [mg] | GRU90-MRP-FTA (product 11-01 from Example 11) [mg] | Peppermi nt oil [µl] |
|---|---|---|---|---|---|---|---|---|
| 17-01 | 50 | 6 | 25 | 25 | - | | | 20 |
| 17-02 | 50 | 6 | 25 | 25 | 100 | | | 20 |
| 17-03 | 50 | 6 | 25 | 25 | | 100 | | 20 |
| 17-04 | 50 | 6 | 25 | 25 | | | 100 | 20 |

Experiment: Each sample was evaluated and average scores from the test panel for each sensory criterium were recorded as the evaluation test results. The resulting taste profiles of the mixtures are shown in Table 17-2.

TABLE 17-2

Sensory evaluations.

| | |
|---|---|
| 17-01 | Sweet, area of cooling: on the tongue and in the throat, slight bitter aftertaste from peppermint flavor |
| 17-02 | Sweeter than sample 17-01, more intensive cooling effect, rounder and smoother peppermint flavor, but less intensive compared to sample 17-01, no bitter aftertaste, area of cooling: on the tongue and then in the throat |
| 17-03 | Sweeter than sample 17-01, more intensive cooling effect, rounder and smoother peppermint flavor, but less intensive compared to sample 17-01, area of cooling: on the tongue and then in the throat |
| 17-04 | Sweeter than sample 17-01, more intensive cooling effect, a little more peppermint flavor, area of cooling: on the tongue and then in the throat |

Conclusion: GRU-MRPs can significantly reduce sweet lingering, enhance sweet and intensive cooling effect of soft candies with peppermint flavor. Meanwhile, it can provide rounder and smoother peppermint flavor, resulting in a better overall likability. The results showed that the taste profile of soft candies with peppermint flavor can be improved by GRU-MRPs

### Example 18 GRU-MRPs Improve the Taste Profile of Menthol Solutions

Raw materials:
Menthol
Peppermint oil
The mixture of GSG-MRP-CA (product 12-01 from Example 12) and GRUds-MRP-FTAs (product 15-02 from Example 15)
GRU90-MRP-FTAs (product 11-02 from Example 11),
GRUds-MRP-FTAs (product 15-02 from Example 15)
GRU90-MRP-FTAs (product 11-01 from Example 11)
GRUds-MRP-FTAs (product 15-01 from Example 15)

Sample preparation:

Preparation of menthol solution: 100 mg of menthol was dissolved in 10 ml of ethanol (puriss) and then diluted 1:400 with distilled water.

The following comparative solutions were prepared:
Product 18-01: Menthol solution
Product 18-02: Menthol solution + 100 ppm the mixture of GSG-MRP-CA (product 12-01 from Example 12) and GRUds-MRP-FTAs (product 15-02 from Example 15)
Product 18-03: Menthol solution + 100 ppm GRU90-MRP-FTAs (product 11-02 from Example 11),
Product 18-04: Menthol solution + 100 ppm GRUds-MRP-FTAs (product 15-02 from Example 15),
Product 18-05: Menthol solution + 100 ppm GRU90-MRP-FTAs (product 11-01 from Example 11),
Product 18-06: Menthol solution + 100 ppm GRUds-MRP-FTAs (product 15-01 from Example 15),
Product 18-07: Menthol solution + 4 % sugar
Product 18-08: Menthol solution + 4 % sugar + 100 ppm the mixture (product 16-01 from Example 16),
Product 18-09: Menthol solution + 4 % sugar + 100 ppm GRU90-MRP-FTAs (product 11-02 from Example 11),
Product 18-10: Menthol solution + 4 % sugar + 100 ppm GRUds-MRP-FTAs (product 15-02 from Example 15),
Product 18-11: Menthol solution + 4 % sugar + 100 ppm GRU90-MRP-FTAs (product 11-01 from Example 11),
Product 18-12: Menthol solution + 4 % sugar + 100 ppm GRUds-MRP-FTAs (product 15-01 from Example 15),

Experiment: Each sample was evaluated and average scores from the test panel for each sensory criterium were recorded as the evaluation test results.

The resulting taste profiles of the mixtures are shown in Table 18-1.

TABLE 18-1

Sensory evaluations:

| Product No. | Solution | Sensory evaluation |
|---|---|---|
| 18-01 | Menthol solution | Fresh, cool, clean, area of cooling: on the tongue and then in the throat. Quick onsite of cooling, medium intensive cooling effect, menthol-like aftertaste |
| 18-02 | Menthol solution + 100 ppm The mixture (product 7-01 from Example 7), | Slight sweeter, more intensive cooling effect, smoother and milder menthol flavor, but less intensive compared to 18-01; area of cooling: on the tongue and then in the throat |
| 18-03 | Menthol solution + 100 ppm GRU90-MRP-FTAs (product 11-02 from Example 11), | Sweeter than sample 2, more intensive cooling effect, smoother and milder menthol flavor, but less intensive compared to 18-01; area of cooling: on the tongue and then in the throat |
| 18-04 | Menthol solution + 100 ppm GRUds-MRP-FTAs (product 15-02 from Example 15), | Slight sweeter, more intensive cooling effect, smoother and milder menthol flavor, but less intensive compared to 18-01; area of cooling: on the tongue and then in the throat |
| 18-05 | Menthol solution + 100 ppm GRU90-MRP-FTAs (product 11-01 from Example 11), | Not sweet, more intensive cooling effect, more intensive menthol flavor compared to 18-01; area of cooling: on the tongue and then in the throat, quick onsite of cooling |
| 18-06 | Menthol solution + 100 ppm GRUds-MRP-FTAs (product 15-01 from Example 15), | Not sweet, more intensive cooling effect, more intensive menthol flavor compared to 18-05 and therefore a little bitter; area of cooling: on the tongue and then in the throat, quick onsite of cooling |
| 18-07 | Menthol solution + 4 % sugar | Sweet, cool, fresh, clean, menthol-like aftertaste, area of cooling: on the tongue and then in the throat, slower onset of cooling intensity compared to 18-01 |
| 18-08 | Menthol solution + 4 % sugar + 100 ppm The mixture (product 7-01 from Example 7), | Sweeter than 18-07, more intensive cooling effect, smoother and milder menthol flavor, but less intensive compared to 18-07; area of cooling: on the tongue and then in the throat |
| 18-09 | Menthol solution + 4 % sugar + 100 ppm GRU90-MRP-FTAs (product 11-02 from Example 11), | Sweeter than 18-08, milder and smoother, cooling intensity and menthol flavor on the same level compared to 18-07; area of cooling: on the tongue and then in the throat |
| 18-10 | Menthol solution + 4 % sugar + 100 ppm GRUds-MRP-FTAs (product 15-02 from Example 15), | Less sweet, less cooling intensity and less menthol flavor compared to 18-08 and 18-09; area of cooling: on the tongue and then in the throat |
| 18-11 | Menthol solution + 4 % sugar + 100 ppm GRU90-MRP-FTAs (product 11-01 from Example 11), | Slightly sweeter than 18-07, more intensive cooling effect, more intensive menthol flavor area of cooling: on the tongue and then in the throat |
| 18-12 | Menthol solution + 4 % sugar + 100 ppm GRUds-MRP-FTAs (product 15-01 from Example 15), | Slightly sweeter than 18-07, more intensive cooling effect, more intensive menthol flavor area of cooling: on the tongue and then in the throat |

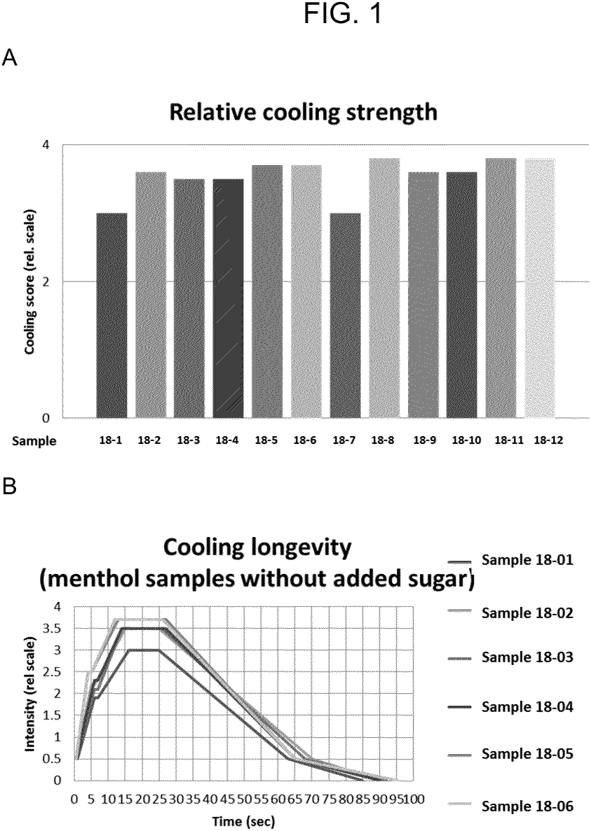

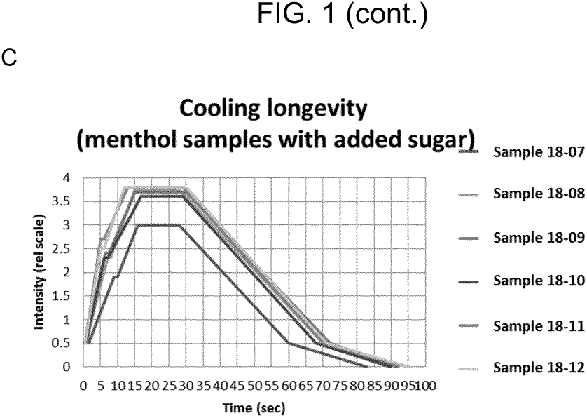

FIG. 1, Panel A, Relative cooling strengths of 18-01 to 18-12.

FIG. 1, Panel B, Cooling longevity of 18-01 to 18-06 (methol samples without added sugar).

FIG. 1, Panel C, Cooling longevity of 18-07 to 18-12 (methol samples with added sugar).

### Example 19. Preparation of Glycosylated Stevioside 90% (GSTV90) From Stevioside 90%

Glycosylated reaction products from stevioside 90% were prepared as outlined below.

Materials: Stevioside 90%, available from EPC Natural Products Co., Ltd. Lot# 20201201; its contents are shown in Table 19-1.

TABLE 19-1

Contents of STV90 (wt%).

| Lot# | RD | RA | STV | RF | RC | DA | RU | RB | STB | TSG(9) |
|---|---|---|---|---|---|---|---|---|---|---|
| 20201201 | / | 2.40 | 91.01 | / | / | / | 0.36 | / | 1.10 | 94.86 |

Process:

i) 15 g β-cyclodextrin was dissolved in 45 ml deionized water.

ii) 15 g stevioside 90% was added to liquefied dextrin.

iii) 0.75 ml CGTase enzyme and 15ml deionized water were added to the mixture in ii) and incubated at 69° C. for 20 hours to glycosylate the STV90 composition via glucose molecules derived from β-cyclodextrin.

iv) The reaction mixture was heated to 85° C. for 10 min to inactivate the CGTase, which was then removed by filtration.

v) The resulting solution of GSTV, residual STV, dextrin and P-cyclodextrinwere decolored and spray dried yielding 25 g of GSTV90 (product 19-01 in Example 19) as a white powder, the contents of which are shown in Table 19-2).

TABLE 19-2

Contents of GSTV90 (wt%)

| | RD | RA | STV | RF | RC | DA | RU | RB | STB | TSG(9) |
|---|---|---|---|---|---|---|---|---|---|---|
| GSTV90 | / | 0.3 | 6.54 | / | / | / | / | / | / | 6.84 |

### Example 20. Preparation of GSTV90-MRP-CA From GSTV90, Xylose And Alanine

Materials: GSTV90: product 19-01 in Example 19.

Process: GSTV90, xylose and alanine and water were weighed and dissolved in water according to Table 20-1. The solutions were then heated at about 100° C. for 2 hours. When the reaction was completed, the solution was filtered through filter paper and the filtrate was dried with a spray dryer, resulting in GSTV90-MRP-CA (product 20-01) as an off white powder.

TABLE 20-1

Sample composition.

| Product No. | Weight of GSTV90 (g) | Weight of xylose (g) | Weight of alanine (g) | Weight of water (g) |
|---|---|---|---|---|
| 20-01 | 8 | 1.5 | 0.5 | 60 |

### Example 21. Sensory Evaluation Comparison of a Natural Sweeteners Sparkling Water Consist of GSTV90 (Product 19-01 in Example 19) with an Artificial Sweeteners Sparkling Water Consist of Sucralose

Materials: GSTV90 (product 19-01 in Example 19); RA100 (RA content, 100.03%); available from Sweet Green Fields Co. Ltd., Lot# CT001-140604; Erythritol available from Zhucheng Dongxiao Biotechnology Co., Ltd., Lot# 201218B.

Process: An artificially sweetened sparkling water containing sucralose was prepared and selected as a reference. A naturally sweetened sparkling water sample containing are GSTV90 (product 19-01 in Example 19) were prepared for comparison. The amounts of these components and others in the samples are shown in Table 21-1.

TABLE 21-1

Preparation of test samples for sensory evaluations.

| Product | Weight of GSTV90 (product 19-01 of example 19) (g) | Weight of sucralose (g) | Weight of Citric Acid (g) | Weight of Sodium citrate (g) | Weight of Erythritol (g) | Weight of RA100 (g) | Volume of deionized water (mL) |
|---|---|---|---|---|---|---|---|
| Reference | - | 0.008 | 0.05 | 0.02 | 3.8 | - | 100 |
| 21-01 | 0.04 | - | 0.05 | 0.02 | 3.8 | 0.015 | 100 |

Each sample was evaluated and average scores from the test panel for each sensory criterium were recorded. The evaluation test results are shown in Table 21-2.

TABLE 21-2

Sensory evaluation results.

| No. | Overall likability | Sweet onset | Sweet peak | Sweet linger | Metallic aftertaste |
|---|---|---|---|---|---|
| Reference | 3 | 3 | 4 | 4 | 4 |
| 21-01 | 4 | 3 | 3.5 | 3 | 2 |

Conclusion: Compared with the artificially sweetened sparkling water containing sucralose, the sweet linger and metallic aftertaste of the naturally sweetened sparkling water containing GSTV90 (product 19-01 of Example 19) was much better. The sensory results observed can be extended to all natural sweeteners. GSTV90 and enzymatically converted products originating from purified stevioside (e.g., 91%, 92%, 93%, 95%, 96%, 97%, 98%, 99%, 99.5%) can be used as sweeteners or flavor. These products can be also combined with other high intensity sweeteners for food and beverage applications.

### Example 22. Sensory Evaluation Comparison of Naturally Sweetened Sparkling Water Containing GSTV90-MRP-CA (Product 20-01 in Example 20) with an Artificially Sweetened Sparkling Water Containing Sucralose

Materials: GSTV90-MRP-CA (product 20-01 in Example 20); RA100 (RA content, 100.03%); available from Sweet Green Fields Co. Ltd., Lot# CT001-140604; Erythritol available from Zhucheng Dongxiao Biotechnology Co., Ltd., Lot# 201218B.

Process: Artificially sweetened sparkling water containing sucralose was selected as a reference. Sparkling water sweetened with a naturally derived sweetener, GSTV90-MRP-CA (product 20-01 in Example 20) was prepared in accordance with Table 22-1.

TABLE 22-1

Test sample preparation for sensory evaluations.

| Product | Weight of GSTV90-MRP-CA (product 20-01 in Example 20) (g) | Weight of sucralose (g) | Weight of Citric Acid (g) | Weight of Sodium citrate (g) | Weight of Erythritol (g) | Weight of RA100 (g) | Volume of deionized water (mL) |
|---|---|---|---|---|---|---|---|
| Reference | - | 0.008 | 0.05 | 0.02 | 3.8 | - | 100 |
| 22-01 | 0.04 | - | 0.05 | 0.02 | 3.8 | 0.015 | 100 |

Each sample was evaluated and average scores from the test panel for each sensory criterium were recorded as the evaluation test results in Table 22-2.

TABLE 22-2

Sensory evaluation results.

| No. | Overall likability | Sweet onset | Sweet peak | Sweet linger | Metallic aftertaste |
|---|---|---|---|---|---|
| Reference | 3 | 3 | 4 | 4 | 4 |
| 22-01 | 4.5 | 3.5 | 4.5 | 1 | 2 |

Conclusion: When compared to sparkling water artificially sweetened with sucralose, sparkling water sweetened with the GSTV90-MRP-CA (product 20-01 in Example 20) had a better sweet onset, sweet peak, sweet linger, and metallic aftertaste. These effects can be applicable to all natural sweeteners.

### Example 23. GSTV90 (Product 19-01 in Example 19) and GSTV90-MRP-CA (Product 20-01 in Example 20) Improve The Taste Profile Of Mouth Wash

Materials: Crest White lock mouth wash (peach flavor): available from Guangzhou P & G Co., Ltd. Lot#:1207D29811.

Ingredients: water, glycerinum, propylene glycol, sodium hexametaphosphate, ethanol, sorbitol, thymol, eucalyptol, methyl salicylate, menthol, benzoic acid, poloxamer 407, sodium benzoate, sodium lauryl sulfate, edible essence, phosphoric acid, saccharin sodium salt, Trichlorogalactose, Cl 17200, and C119140.

Process: Concentrated GSTV90 (product 19-01 in Example 19) and GSTV90-MRP-CA (product 20-01 in Example 20) were each dissolved in mouth wash in the amounts shown in Table 23-1.

TABLE 23-1

Sample compositions.

| Type of added products | Weight of added products (mg) | Volume of mouth wash (mL) | Concentration (ppm) |
|---|---|---|---|
| Base | - | 100 | - |
| GSTV90 (product 19-01 in Example 19) | 5 | 100 | 50 |
| GSTV90-MRP-CA (product 20-01 in Example 20) | 5 | 100 | 50 |

Experiment: Each sample was evaluated according to the aforementioned sensory evaluation method. Average scores from the panel for each sensory criterium were taken were determined. The resulting taste profiles of the test samples are shown in Table 23-2.

TABLE 23-2

Sensory evaluation results.

| | Overall likability | Coolness | Refreshing | Bitterness | Flavor |
|---|---|---|---|---|---|
| Base | 2.5 | 2.5 | 3 | 3 | 3 |
| GSTV90 | 3 | 3 | 3.5 | 1 | 4 |
| GSTV90-MRP-CA | 3.5 | 3.5 | 4 | 1.5 | 3.5 |

Conclusion: Both GSTV90 (product 19-01 in Example 19) and GSTV90-MRP-CA (product 20-01 in Example 20) can improve the taste profile of Crest White lock mouth wash. Specifically, GSTV90 and GSTV90-MRP-CA can reduce the unpleasant bitterness, and improve the peach flavor and coolness of the mouth wash. These effects can be extend to all natural sweeteners.

### Example 24. GSTV90 (Product 19-01 in Example 19) and GSTV90-MRP-CA (Product 20-01 in Example 20) Improve the Coolness of Essential Balm

Materials: essential balm, (liquid state), available from Shanghai Zhonghua Pharmaceutical Nantong Co., Ltd.. Lot#:C200813

Ingredients: menthol, methyl salicylate, camphor, eucalyptus oil, eugenol, essence, chlorophyll, and liquid paraffin

Process: GSTV90 (product 19-01 in Example 19) and GSTV90-MRP-CA (product 20-01 in Example 20) were dissolved in the essential balm in the amounts shown in Table 24-1.

TABLE 24-1

Sample compositions.

| | | | | |
|---|---|---|---|---|
| Sample | Weight of GSTV90 (product 19-01 in Example 19) | Weight of GSTV90-MRP-CA (product 20-01 in Example 20) | Volume of essential balm | Evaluation type |
| Essential balm | - | | 100 mL | Skin smear |
| Essential balm + GSTV90 (product 19-01 in Example 19) | 10 mg | | 100 mL | |
| Essential balm + GSTV90-MRP-CA (product 20-01 in Example 20) | | 10 mg | 100 mL | |

Experiment: Each sample was evaluated for the feeling of coolness by a skin smear. The sensory evaluation results from the samples are shown in Table 24-2.

TABLE 24-2

Sensory evaluation results.

| | | |
|---|---|---|
| Sample | Evaluation type | Sensory evaluation |
| Essential balm | Skin smear | GSTV90 (product 19-01 in Example 19) can improve coolness onset, coolness intensity and coolness duration of essential balm |
| Essential balm + GSTV90 (product 19-01 in Example 19) | | |
| Essential balm + GSTV90-MRP-CA (product 20-01 in Example 20) | | GSTV90-MRP-CA (product 20-01 in Example 20) can improve coolness intensity and coolness duration of essential balm. |

Conclusion: Both GSTV90 (product 19-01 in Example 19) and GSTV90-MRP-CA (product 20-01 in Example 20) can improve coolness onset, coolness intensity and coolness duration of essential balm. These results can extend to stevia extracts, glycosylated stevia extracts and their MRPs.

### Example 25. The Influence of GRU90-MRP-FTA (Product 2-02 in Example 2) on the Stable Expression of TRPM8 Channel Current by Whole-Cell Patch Clamp Technique

Material: GRU90-MRP-FTA, the product 2-02 in Example 2

### I. Sample Preparation

i) GRU90-MRP-FTA (product 2-02 in example 2) are directly prepared to 10000 ppm working solution with extracellular solution.

ii) 10000 ppm working solution of the subject was diluted in turn with extracellular solution to prepare 1000 ppm, 100 ppm and 10 ppm liquids.

iii) Visually inspect the solubility of the sample to be tested. GRU90-MRP-FTA (product 2-02 in example 2) is completely dissolved without visible precipitation.

### Cell Culture

CHO-K1 cell line (TRPM8 stably expressed) is adopted in this experiment and the gene information: transient receptor potential channel subfamily m member, nm _ 024080. It was cultured in F12 medium containing 10% fetal bovine serum at 37° C. and 5% carbon dioxide.

Cell passage: old culture medium is removed and washed with PBS once time, and then1 mL Trypsin-EDTA solution are added and incubate at 37° C. for about 1 min. When the cells are detached from the bottom of the dish, 5 ml of complete medium is added and preheated at 37° C. Gently blow the cell suspension with a straw to separate the aggregated cells. Transfer the cell suspension to a sterile centrifuge tube, and centrifuge at 1000 rpm for 5 min to collect cells. The cells are cultured in a 6 cm cell culture dish, and the amount of cells inoculated in each cell culture dish is 2.5* 10⁵ cells (final volume: 5 mL).

In order to maintain the electrophysiological activity of cells, the cell density must not exceed 80%.

Patch clamp test: Before the test, the cells were separated by Trypsin-EDTA. 8*10³ cells were spread on the cover glass, cultured in 24-well plate (final volume: 500 µL), and induced by tetracycline for 18 hours, and then the test was carried out.

All operations are followed the standard operating procedure of cell culture of Beijing ICE BIOSCIENCE CO.Ltd.

### Electrophysiological Records

1. Record the liquid used.

Extracellular fluid: K-007-1: 140 mM NaCl, 3.5 mM KCl, 1 mM MgCl₂·6H₂O, 2 mM CaCl_{2•}2H₂O, 10 mM D-Glucose, 10 mM HEPES , 1.25 mM NaH₂PO_{4•}H₂O, NaOH adjust the pH to 7.4.

Intracellular fluid: Nav-001-2: 50 mM CsCl, 20 mM EGTA, 10 mM NaCl, 10 mM HEPES, 60 mM CsF, CsOH adjust the pH to 7.2

The storage time of extracellular fluid is 2 weeks. Intracellular fluid is prepared and packed into 1 mL each tube and then frozen in the refrigerator at -20° C. And the newly melted intracellular fluid is used every day. It’s worth noting that all intracellular fluid should be used up in three months. After three months, discard the old intracellular fluid and re-prepare it.

### Patch Clamp Detection

The voltage stimulation scheme of whole-cell patch clamp recording whole-cell TRPM8 current is as follows: when the whole-cell seal is formed, the cell membrane voltage is clamped at -80 mV. Record the voltage step from -80 mV to -100 mV, and then give 500 ms Ramp stimulation to 100 mV. Data were collected repeatedly every 5 s to observe the effect of drugs on the peak value of outward current of TRPM8. Test data are collected by EPC-10 amplifier (HEKA) and stored in PatchMaster(HEKA) software.

The capillary glass tube is drawn into a recording electrode by a microelectrode drawing instrument. Manipulate the microelectrode manipulator under the inverted microscope to contact the recording electrode to the cell, and give negative pressure suction to form G ω seal. After G ω sealing is formed, rapid capacitance compensation is performed, and then negative pressure is continuously applied to suck the cell membrane, thus forming a whole-cell recording mode. Then, the compensation of slow capacitance is made and the film capacitance and series resistance are recorded. No leakage compensation is given.

The cover glass covered with cells is placed in the recording bath of inverted microscope, and the test compound and the external liquid without compound flow through the recording bath by gravity perfusion to act on the cells, and the liquid exchange is carried out by vacuum pump during recording. Each cell used TRPM8 current after 100 µM menthol as its own control group. Two cells were detected independently. All electrophysiological tests were conducted at room temperature.

### Data Quality Standard

The following criteria are used to judge whether the data is acceptable:

1. Electrode resistance < 5 MΩ.

2. Sealing resistance > 1 GΩ ω.

Start of access resistance < 15mω.

End of access resistance < 15mω

### Data Analysis

Excitement:

The TRPM8 channel current after each drug concentration and the TRPM8 current after 100 µM menthol are standardized

$$\left(\frac{P e a k c u r r e n t c o m p o u n d}{P e a k c u r r e n t v e h i c l e}\right)$$

. Calculate the mean, standard deviation and standard error.

Regulating effect:

Standardize the current of TRPM8 channel after drug treatment and TRPM8 current after 100 µM menthol treatment

$$( \frac{P e a k c u r r e n t c o m p o u n d}{P e a k c u r r e n t v e h i c l e} )$$

. Then vehicle calculate the corresponding regulating rate of drugs

$$\left(1 - \frac{P e a k c u r r e n t c o m p o u n d}{P e a k c u r r e n t v e h i c l e}\right)$$

, and calculate the mean, standard deviation and standard error.

### Data

i) Excitatory action

The Excitatory action is performed on cell S6-211013001, S6-211013002, S6-211013003 and S6-211013004 with different concentration of GRU90-MRP-FTA (product 2-02 in Example 2) sample. The results are shown in Table 25-1.

TABLE 25-1

The Excitatory action of GRU90-MRP-FTA (product 2-02 in Example 2) sample on TRPM8 channel current

| | TRPM8-CHO Peak Current (pA) | | | | | | % of menthol 100 µM | | | |
|---|---|---|---|---|---|---|---|---|---|---|
| Cell No. | Contro 1 | sample 10 ppm | sampl e 100 ppm | sampl e 1000 ppm | sampl e 10000 ppm | Menthol 100 µM | sample 10 ppm | sample 100 ppm | sample 1000 ppm | sample 10000 ppm |
| S6-211013001 | 275.76 | 122.58 | | | | 2135.50 | -8.24% | | | |
| S6-211013002 | 86.77 | | 32.28 | | | 2135.50 | | -2.66% | | |
| S6-211013003 | 248.53 | | | 187.91 | 160.75 | 2067.70 | | | -3.33% | -4.83% |
| S6-211013004 | 174.54 | | | 207.47 | 175.28 | 3724.60 | | | 0.93% | 0.02% |
| mean | 196.40 | 122.58 | 32.28 | 197.69 | 168.02 | 2515.83 | -8.24% | -2.66% | -1.20% | -2.40% |
| SD | 84.68 | NA | NA | 13.83 | 10.27 | 806.48 | NA | NA | 3.01% | 3.43% |
| n | four | one | one | 2 | 2 | four | one | one | 2 | 2 |
| SE | 42.34 | NA | NA | 9.78 | 7.27 | 403.24 | NA | NA | 2.13% | 2.42% |

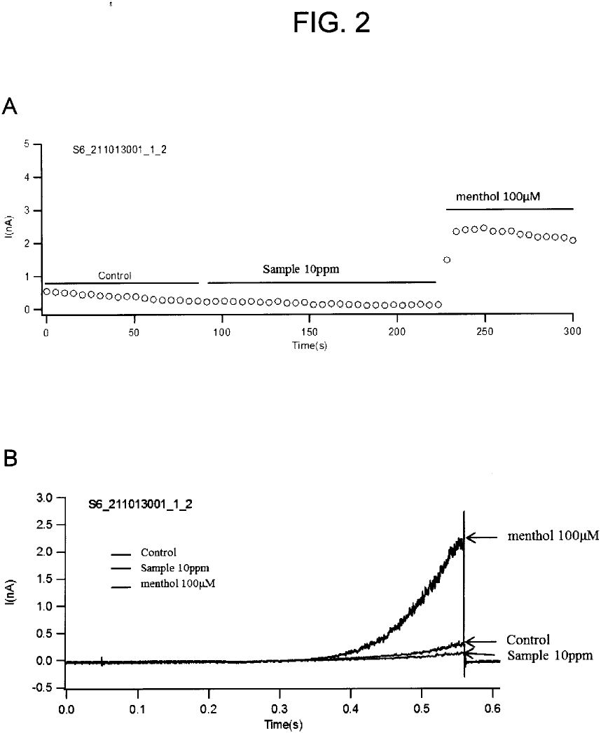

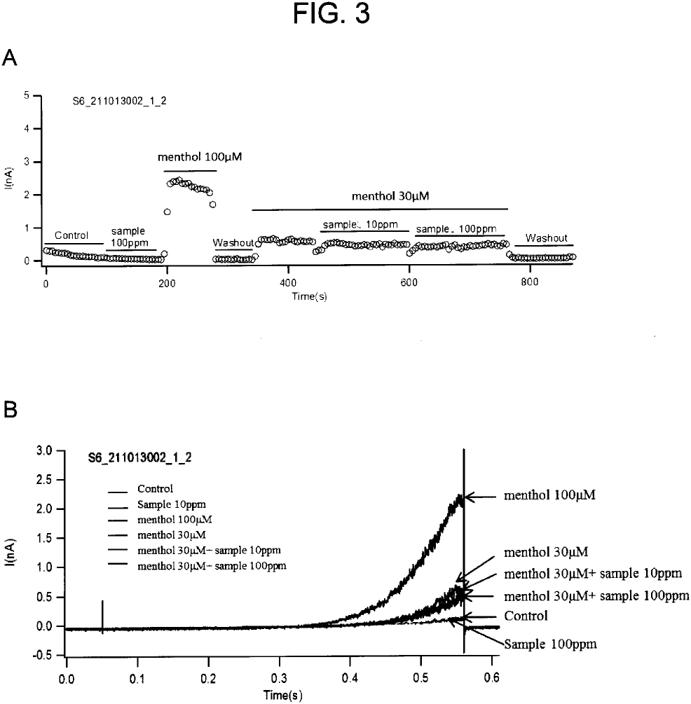

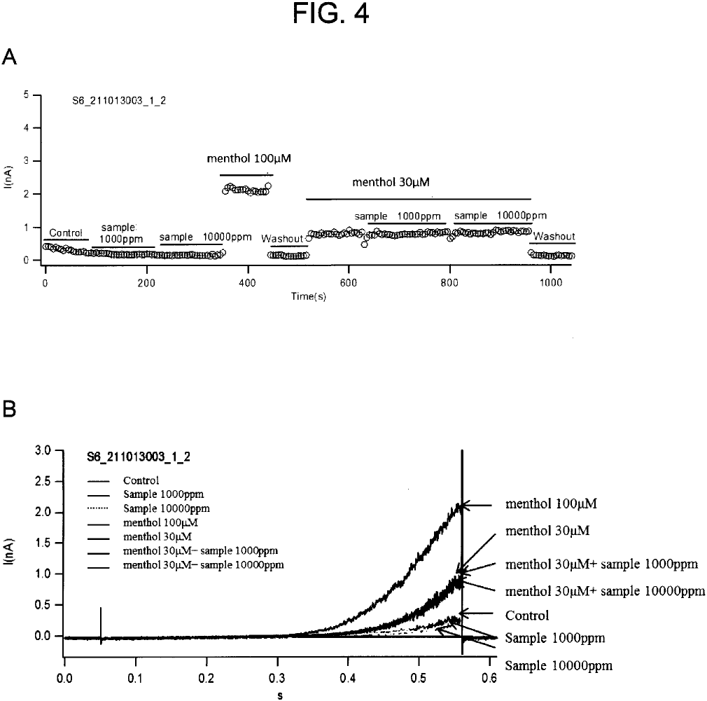

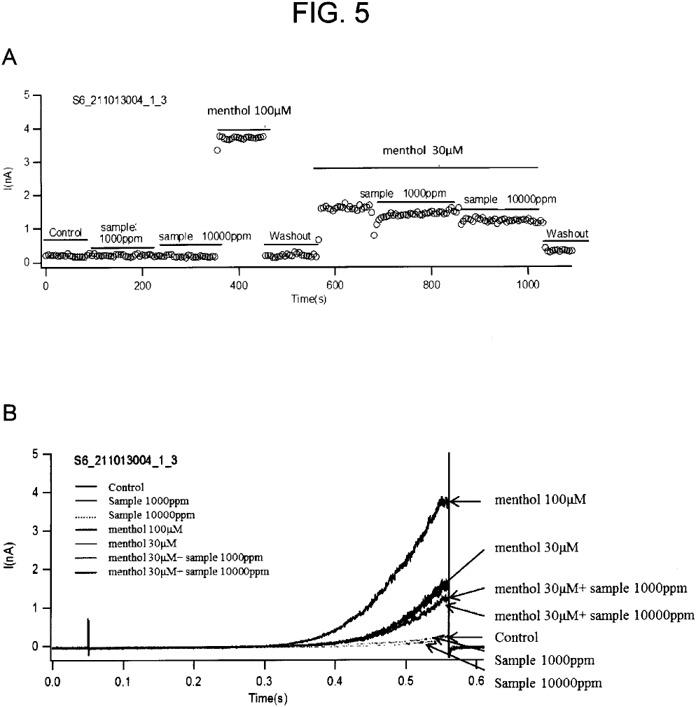

FIGS. 2-5 show the excitatory action of different concentration GRU90-MRP-FTA (product 2-02 in Example 2) sample on TRPM8 channel current on cell S6-211013001 (FIG. 2), cell S6-211013002 (FIG. 3), cell S6-211013003 (FIG. 4) and cell S6-211013004 (FIG. 5).

ii) Regulating action

The regulating action is performed on cell S6-211013002, S6-211013003 and S6-211013004 with different concentration of GRU90-MRP-FTA (product 2-02 in Example 2) sample. The results are shown in Table 25-2.

TABLE 25-2

The regulating action of GRU90-MRP-FTA (product 2-02 in Example 2) sample on TRPM8 channel current

| | TRPM8-CHO Peak Current(pA) | | | | | | Regulating Effect% | | | |
|---|---|---|---|---|---|---|---|---|---|---|
| Cell No. | Contro 1 | Mentho 130 µM | Mentho 130 µM + sample 10 ppm | Mentho 130 µM + sample 100pp m | Menthol 30 µM + sample 1000pp m | Menthol 30 µM + sample 10000pp m | Menth ol 30 µM + sample 10 ppm | Menth ol 30 µM + sample 100pp m | Menth ol 30 µM + sample 1000p pm | Menthol 30 µM + sample 10000 ppm |
| S6-21101 3002 | 86.77 | 569.97 | 448.82 | 448.28 | | | 25.07 % | 25.18 % | | |
| S6-21101 3003 | 248.53 | 826.03 | | | 825.39 | 871.86 | | | 0.11% | -7.94% |
| S6-21101 3004 | 174.54 | 1634.1 0 | | | 1503.90 | 1213.20 | | | 8.92% | 28.84% |
| mean | 169.95 | 1010.0 3 | 448.82 | 448.28 | 1164.65 | 1042.53 | 25.07 % | 25.18 % | 4.52% | 10.45% |
| SD | 80.98 | 555.42 | NA | NA | 479.78 | 241.36 | NA | NA | 6.23% | 26.00% |
| n | three | three | one | one | 2 | 2 | one | one | 2 | 2 |
| SE | 46.75 | 320.67 | NA | NA | 339.26 | 170.67 | NA | NA | 4.40% | 18.39% |

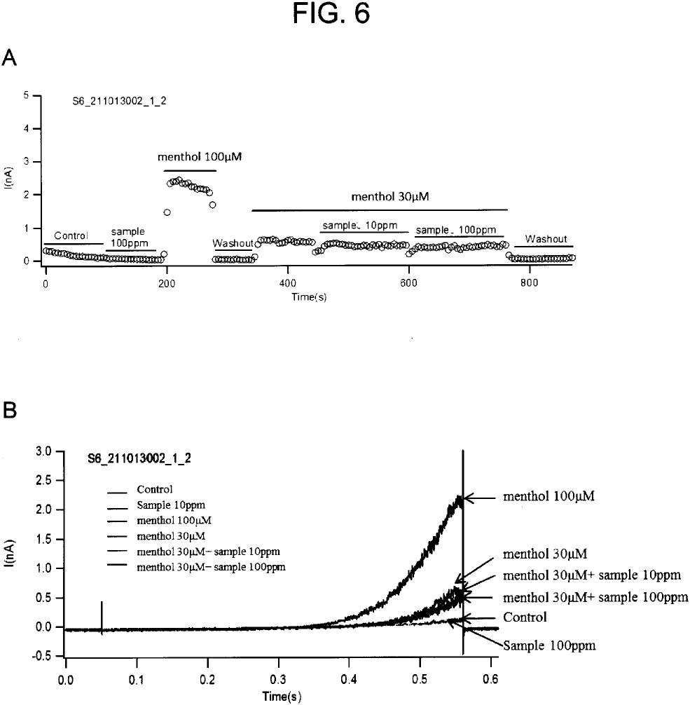

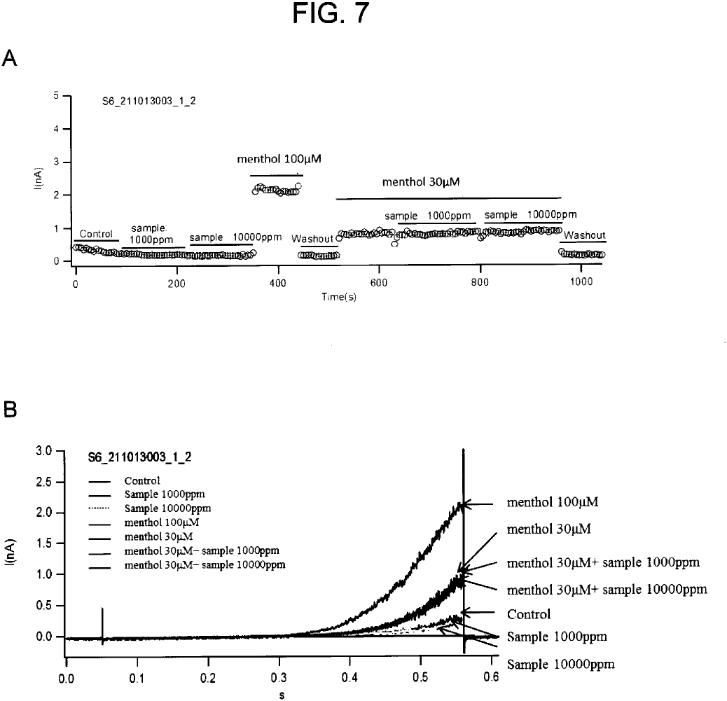

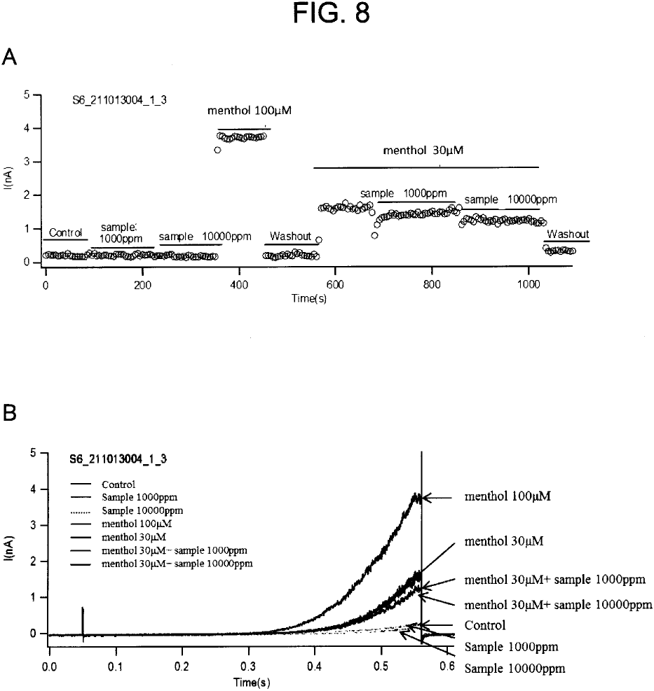

FIGS. 6-8 show the regulating action of different concentration GRU90-MRP-FTA (product 2-02 in Example 2) sample on TRPM8 channel current on cell S6-211013002 (FIG. 6), cell S6-211013003 (FIG. 7) and cell S6-211013004 (FIG. 8).

Result evaluation

In this study, patch clamp technique was used to detect the excitability and regulating effect of GRU90-MRP-FTA (the product 2-02 in Example 2) on TRPM8 channel, so as to evaluate the influence of TRPM8 channel. The test results are summarized as follows:

Results of excitation of GRU90-MRP-FTA (the product 2-02 in example 2) on TRPM8 current:

| Compound | % of menthol 100 µM | | | | n |
|---|---|---|---|---|---|
| | 10 ppm | 100 ppm | 1000 ppm | 10000 ppm | |
| GRU90-MRP-FTA (product 2-02 in Example 2) | -8.24% | -2.66% | -1.20%±2.13% | -2.4%±2.42% | 2 |

The results showed that the GRU90-MRP-FTA (the product 2-02 in example 2) had no direct excitatory effect on TRPM8.

Results of regulating effect of GRU90-MRP-FTA (the product 2-02 in example 2) on TRPM8 current:

| Compound | Regulating Effect% | | | | n |
|---|---|---|---|---|---|
| | 10 ppm | 100 ppm | 1000 ppm | 10000 ppm | |
| GRU90-MRP-FTA (product 2-02 in Example 2) | 25.07% | 25.18% | 4.52%±4.40 % | 10.45%±18.39 % | 2 |

Conclusion: The results showed that the GRU90-MRP-FTA (the product 2-02 in Example 2) had no direct excitatory effect on TRPM8. However, GRU90-MRP-FTA (the product 2-02 in Example 2) had regulating effect on TRPM8. This may explain the influence of GRU90-MRP-FTA (the product 2-02 in Example 2) on cool feeling.

### Example 26. The Influence of GRU90-MRP-FTA (Product 2-02 in Example 2) on the Stable Expression of TRPM4 Channel Current by Whole-Cell Patch Clamp Technique

Material: GRU90-MRP-FTA, the product 2-02 in Example 2.

### Sample Preparation

i) GRU90-MRP-FTA (product 2-02 in Example 2) are directly prepared to 0.01 g /mL working solution with extracellular solution.

ii) Visually inspect the solubility of the sample to be tested. GRU90-MRP-FTA (product 2-02 in Example 2) is completely dissolved without visible precipitation.

### Cell Culture

HEK-293 cells (TRPM4/SUR1 stably expressed) is adopted in this experiment and the gene information: trpm4: nm _ 017636; SUR1, ABCC8: NM_000352.

Day 1: HEK293 cells were transferred to 6-well plates, with about 5× 10⁵ cells per well.

Day 2: Lipofectamine 3000 transfection reagent was used for transfection, and the ratio of plasmid to transfection reagent was 1 µg: 2 µL. The total dosage of plasmids per well is 3 µg, and the ratio of TRPM4 and SUR1 plasmids is 1:10. Specifically, take two sterile centrifuge tubes, each add 100 µL Opti-MEM. And then one tube add 6 µL Lipofectamine 3000, the other tube add 0.2 µg TRPM4 plasmid, 2.2 µg SUR1 plasmid and 6 µL Lipofectamine 3000, and mix well. Then add the diluted plasmid DNA into diluted Lipofectamine 3000, and incubate at room temperature for 10-15 min. Drop DNA- liposome complex into cells, gently shake and mix, and put into an incubator for culture. Change the fluid after 4-6 hours.

Day 3: Digest the cells and inoculate them into 24-well plates with cover glass in advance, with 8×10³ cells per well.

Day 4: Patch clamp testing.

All operations are followed the standard operating procedure of cell culture of Beijing ICE BIOSCIENCE CO.Ltd.

### Electrophysiological Records

1. Record the liquid used

Extracellular fluid:

K-007-1: 140 mM NaCl, 3.5 mM KCl, 1 mM MgCl₂·6H₂O, 2 mM CaCl₂-2H₂O, 10 mM D-Glucose, 10 mM HEPES, 1.25 mM NaH₂PO₄-H₂O, NaOH adjust the pH to 7.4.

Intracellular fluid:

Nav-001-2: 50 mM CsCl, 20 mM EGTA, 10 mM NaCl, 10 mM HEPES, 60 mM CsF, CsOH adjust the pH to 7.2.

The storage time of extracellular fluid is 2 weeks. Intracellular fluid is prepared and packed into 1 mL each tube and then frozen in the refrigerator at -20° C. And the newly melted intracellular fluid is used every day. It’s worth noting that all intracellular fluid should be used up in three months. After three months, discard the old intracellular fluid and re-prepare it.

### Patch Clamp Detection

The voltage stimulation scheme of whole-cell patch clamp recording whole-cell TRPM4/SUR1 current is as follows: when the whole-cell seal is formed, the cell membrane voltage is clamped at -80 mV. Record the voltage step from -80 mV to -100 mV, and then give 500 ms Ramp stimulation to 100 mV. Data were collected repeatedly every 5 s to observe the effect of drugs on the peak value of outward current of TRPM4/SUR1. Test data are collected by EPC-10 amplifier (HEKA) and stored in PatchMaster(HEKA) software.

The capillary glass tube is drawn into a recording electrode by a microelectrode drawing instrument. Manipulate the microelectrode manipulator under the inverted microscope to contact the recording electrode to the cell, and give negative pressure suction to form G ω seal. After G ω sealing is formed, rapid capacitance compensation is performed, and then negative pressure is continuously applied to suck the cell membrane, thus forming a whole-cell recording mode. Then, the compensation of slow capacitance is made and the film capacitance and series resistance are recorded. No leakage compensation is given.

The cover glass covered with cells is placed in the recording bath of inverted microscope, and the test compound and the external liquid without compound flow through the recording bath by gravity perfusion to act on the cells, and the liquid exchange is carried out by vacuum pump during recording. The TRPM4/SUR1 current of the whole cell was recorded, and each cell used the TRPM4/SUR1 current after 10 µM A23187 as its own control group. Two cells were detected independently. All electrophysiological tests were conducted at room temperature.

### Data Quality Standard

The following criteria are used to judge whether the data is acceptable:

1. Electrode resistance < 5 MΩ.

2. Sealing resistance > 1 GΩ ω.

3. Start of access resistance < 15 mω.

4. End of access resistance < 15 mω.

### Data Analysis

Excitement:

Standardize the TRPM4/SUR1 channel current after each drug concentration and TRPM4/SUR1 current after 10 µMA23187

$$\left(\frac{P e a k c u r r e n t c o m p o u n d}{P e a k c u r r e n t v e h i c l e}\right) .$$

Calculate the mean, standard deviation and standard error.

Regulating effect:

Standardize the TRPM4/SUR1 channel current and A23187 10 µM current after drug treatment

$$\left(\frac{P e a k c u r r e n t c o m p o u n d}{P e a k c u r r e n t v e h i c l e}\right) .$$

Then calculate the corresponding regulating rate of drugs

$$\left(1 - \frac{P e a k c u r r e n t c o m p o u n d}{P e a k c u r r e n t v e h i c l e}\right) ,$$

and calculate the mean, standard deviation and standard error.

### Data

i) Excitatory action

The Excitatory action is performed on cell S6-211208007 and cell S6-211208010 with 0.01 g/mL GRU90-MRP-FTA (product 2-02 in Example 2) sample. The results are shown in Table 26-1.

TABLE 26-1

The Excitatory action of GRU90-MRP-FTA (product 2-02 in Example 2) sample on TRPM4 channel current

| | TRPM4-HKE Peak Current(pA) | | | % of A23187 10 µM |
|---|---|---|---|---|
| Cell No. | Control | sample 0.01 g/mL | A23187 10 µM | sample 0.01 g/mL |
| S6-211208007 | 290.99 | 261.17 | 1225.00 | -3.19% |
| S6-211208010 | 75.36 | 72.22 | 572.15 | -0.63% |
| mean | 183.17 | 166.70 | 898.58 | -1.91% |
| SD | 152.48 | 133.61 | 461.63 | 1.81% |
| n | 2 | 2 | 2 | 2 |
| SE | 107.82 | 94.47 | 326.43 | 1.28% |

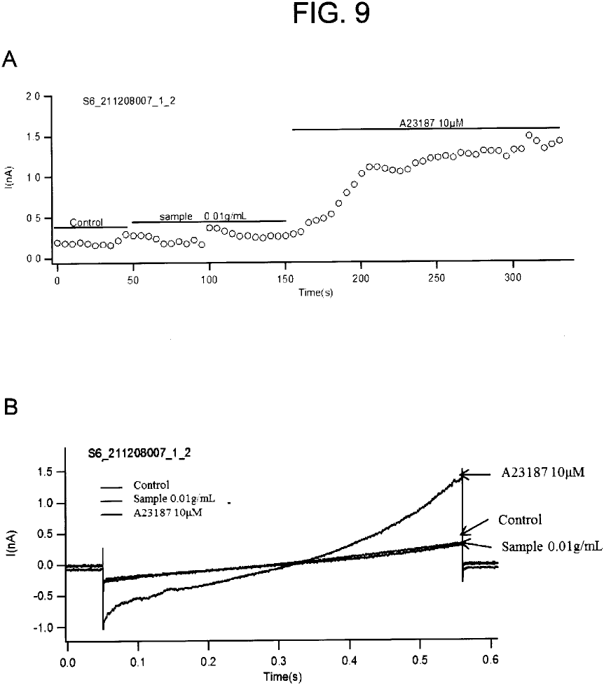

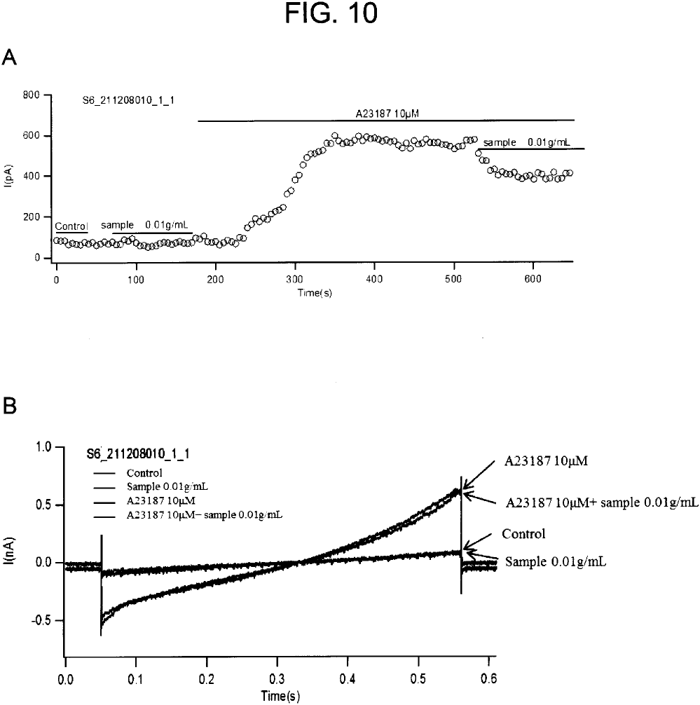

FIGS. 9-10 show the excitatory action of different concentration GRU90-MRP-FTA (product 2-02 in Example 2) sample on TRPM4 channel current on cell S6-211208007 (FIG. 9) and cell S6-211208010 (FIG. 10).

i) Regulating action

The regulating action is performed on cell S6-211208008 and cell S6-211208010 with 0.01 g/mL GRU90-MRP-FTA (product 2-02 in Example 2) sample. The results are shown in Table 26-2.

TABLE 26-2

The regulating action of GRU90-MRP-FTA (product 2-02 in Example 2) sample on TRPM4 channel current.
Sample

| | TRPM4-HKE Peak Current(pA) | | | Regulating Effect% |
|---|---|---|---|---|
| Cell No. | Control | A23187 10 µM | A23187 10 µM+sample 0.01 g/mL | A23187 10 µM+sample 0.01 g/mL |
| S6-211208008 | 244.58 | 9017.10 | 7496.50 | 17.33% |
| S6-211208010 | 75.355 | 572.15 | 535.42 | 7.39% |
| mean | 159.97 | 4794.63 | 4015.96 | 12.36% |
| SD | 119.66 | 5971.48 | 4922.23 | 7.03% |
| n | 2 | 2 | 2 | 2 |
| SE | 84.61 | 4222.48 | 3480.54 | 4.97% |

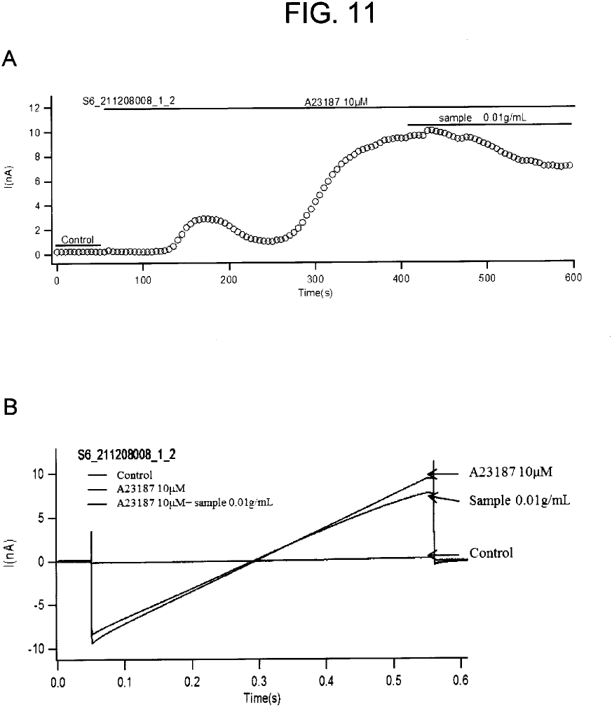

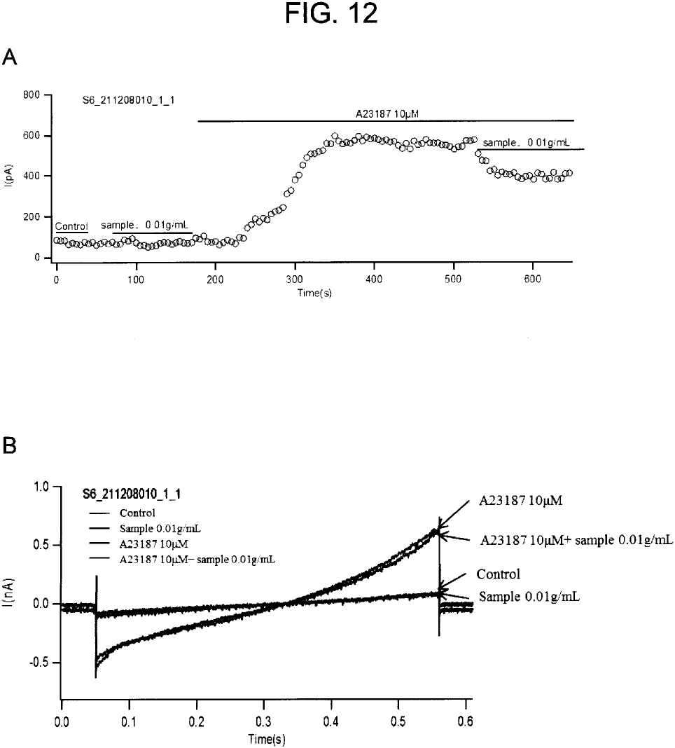

FIGS. 11 and 12 show the regulating action of different concentration GRU90-MRP-FTA (product 2-02 in Example 2) sample on TRPM4 channel current on cell S6-211208008 (FIG. 11) and cell S6-211208010 (FIG. 12).

Result evaluation

In this study, patch clamp technique was used to detect the excitability and regulating effect of GRU90-MRP-FTA (the product 2-02 in Example 2) on TRPM4 channel, so as to evaluate the influence of TRPM4 channel. The test results are summarized as follows:

Results of excitation of GRU90-MRP-FTA (the product 2-02 in Example 2) on TRPM4 current:

| Compound | % of A23187 10 µM | n |
|---|---|---|
| GRU90-MRP-FTA (the product 2-02 in Example 2) | -1.91%±1.28% | 2 |

The results showed that the GRU90-MRP-FTA (the product 2-02 in Example 2) had no direct excitatory effect on TRPM4.

Results of regulating effect of GRU90-MRP-FTA (the product 2-02 in Example 2) on TRPM4 current:

| Compound | Regulating effect%-0.01 g/mL | n |
|---|---|---|
| GRU90-MRP-FTA (the product 2-02 in Example 2) | 12.36%±4.97% | 2 |

Conclusion: The results showed that the GRU90-MRP-FTA (the product 2-02 in Example 2) had no direct excitatory effect on TRPM4. However, GRU90-MRP-FTA (the product 2-02 in Example 2) had regulating effect on TRPM4. This may explain the influence of GRU90-MRP-FTA (the product 2-02 in Example 2) on cool feeling.

### Example 27. The Influence of GRU90-MRP-FTA (Product 2-02 In Example 2) on the Stable Expression of TRPV3 Channel Current by Whole-Cell Patch Clamp Technique

Material. GRU90-MRP-FTA, the product 2-02 in example 2

### Sample Preparation

i) GRU90-MRP-FTA (product 2-02 in example 2) are directly prepared to 0.01 g /mL working solution with extracellular solution.

ii) Visually inspect the solubility of the sample to be tested. GRU90-MRP-FTA (product 2-02 in example 2) is completely dissolved without visible precipitation.

### Cell Culture

HEK293 cells (TRPV3 stably expressed) is adopted in this experiment and TRPV3 receptor gene information: transient receptor potential channel subfamily vmember, nm_ 145068.

Day 1: HEK293 cells were transferred to 6-well plates, with about 5×105 cells per well.

Day 2: Lipofectamine 3000 transfection reagent was used for transfection, and the ratio of plasmid to transfection reagent was 1 µg: 2 µL. The total dosage of plasmid per well is 3 µg. Specifically, take two sterile centrifuge tubes, each add 100 µL Opti-MEM. And then one tube add 6 µL Lipofectamine 3000, the other tube add TRPV3 plasmid, 6 µL Lipofectamine 3000 and mix well. Then add the diluted plasmid DNA into diluted Lipofectamine 3000, and incubate at room temperature for 10-15 min. Drop DNA- liposome complex into cells, gently shake and mix, and put into an incubator for culture. Change the fluid after 4-6 hours.

Day 3: Digest the cells and inoculate them into 24-well plates with cover glass in advance, with 8×103 cells per well.

Day 4: Patch clamp test.

All operations follow the standard operating procedure of cell culture of Beijing ICE BIOSCIENCE CO.Ltd.

### Electrophysiological Records

1. Record the liquid used

Extracellular fluid:

TRPV3-001-1: 140 mM NaCl, 3.5mMKCl, 1 mM MgCl_{2•}6H₂O, 2 mM CaCl₂-2H₂O, 10 mM D-Glucose, 10 mM HEPES, 1.25 mM NaH₂PO₄-H₂O, NaOH adjust the pH to 7.4.

Intracellular fluid:

TRPV3-001-2: 50 mM CsCl, 20 mM EGTA, 10 mM NaCl, 10 mM HEPES, 60 mM CsF, CsOH adjust the pH to 7.2.

The storage time of extracellular fluid is 2 weeks. Intracellular fluid is prepared and packed into 1 mL each tube and then frozen in the refrigerator at -20° C. And the newly melted intracellular fluid is used every day. It’s worth noting that all intracellular fluid should be used up in three months. After three months, discard the old intracellular fluid and re-prepare it.

### Patch Clamp Detection

The voltage stimulation scheme of whole-cell patch clamp recording whole-cell TRPV3 current is as follows: when the whole-cell seal is formed, the cell membrane voltage is clamped at -80 mV. Record the voltage step from 0 mV to -100 mV for 20 ms, and then 100 ms Ramp was given to stimulate to 100 mV. Data were collected repeatedly every 5 s to observe the effect of drugs on the peak value of outward current of TRPV3. Test data are collected by EPC-10 amplifier (HEKA) and stored in PatchMaster (HEKA) software.

### Data Quality Standard

The following criteria are used to judge whether the data is acceptable:

1. Electrode resistance < 5 MΩ.

2. Sealing resistance > 1 GΩ ω.

3. Start of access resistance < 15mω.

4. End of access resistance < 15mω.

### Data Analysis

Excitement: The TRPV3 channel current after each drug concentration and the TRPV3 current after 2 mM menthol are standardized

$$\left(\frac{P e a k c u r r e n t c o m p o u n d}{P e a k c u r r e n t v e h i c l e}\right) .$$

Calculate the mean, standard deviation and standard error.

Regulating effect: Standardize the current of TRPV3 channel after drug treatment and TRPV3 current after 2 mM menthol treatment

$$\left(\frac{P e a k c u r r e n t c o m p o u n d}{P e a k c u r r e n t v e h i c l e}\right) .$$

Then calculate the corresponding Regulating rate of drugs

$$\left(1 - \frac{P e a k c u r r e n t c o m p o u n d}{P e a k c u r r e n t v e h i c l e}\right) ,$$

and calculate the mean, standard deviation and standard error.

### Data

i) Excitatory action

The Excitatory action is performed on cell S6-211208003 and cell S6-211208004 with 0.01 g/mL GRU90-MRP-FTA (product 2-02 in Example 2) sample. The results are shown in Table 27-1.

TABLE 27-1

The Excitatory action of GRU90-MRP-FTA (product 2-02 in Example 2) sample on TRPV3 channel current
Sample

| | TRPV3-HKE Peak Current(pA) | | | % of menthol 2 mM |
|---|---|---|---|---|
| Cell No. | Control | sample 0.01 g/mL | Menthol 2 mM | sample 0.01 g/mL |
| S6-211208003 | 599.65 | 141.58 | 5519.50 | -9.31% |
| S6-211208004 | 185.90 | 116.88 | 4980.60 | -1.44% |
| mean | 392.78 | 129.23 | 5250.05 | -5.38% |
| SD | 292.57 | 17.47 | 381.06 | 5.57% |
| n | 2 | 2 | 2 | 2 |
| SE | 206.88 | 12.35 | 269.45 | 3.94% |

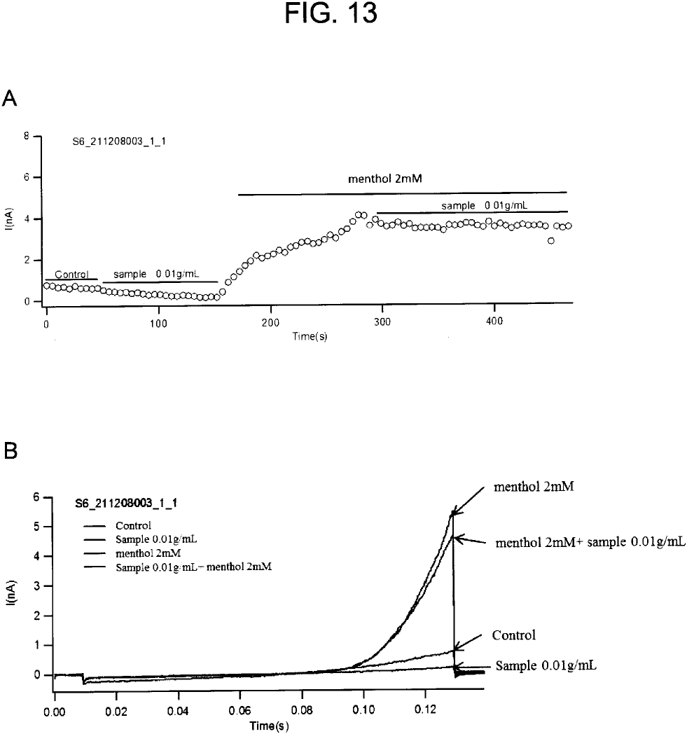

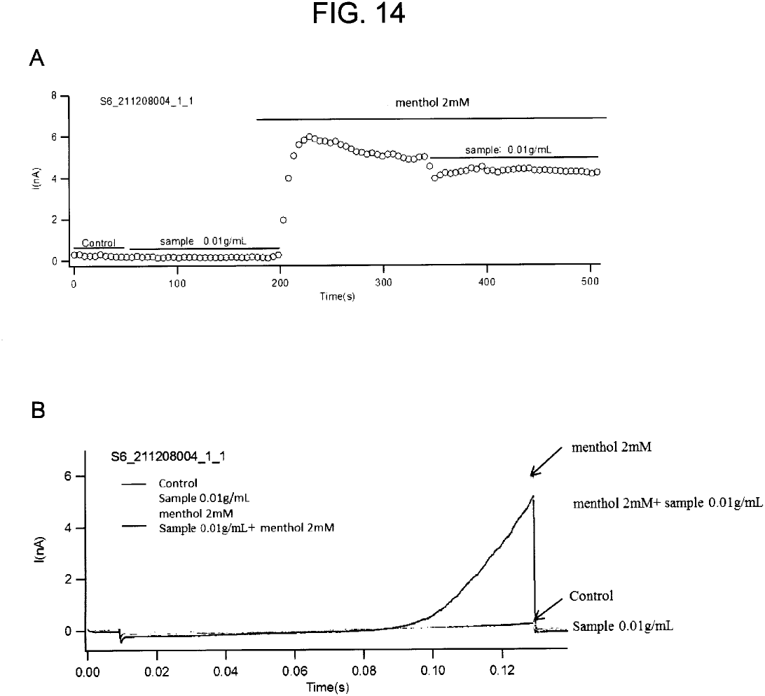

FIGS. 13 and 14 show the excitatory action of different concentration GRU90-MRP-FTA (product 2-02 in Example 2) sample on TRPV3 channel current on cell S6-211208003 (FIG. 13) and cell S6-211208004 (FIG. 14).

i) Regulating action

The regulating action is performed on cell S6-211208003 and cell S6-211208004 with 0.01 g/mL GRU90-MRP-FTA (product 2-02 in Example 2) sample. The results are shown in Table 27-2.

TABLE 27-2

The regulating action of GRU90-MRP-FTA (product 2-02 in Example 2) sample on TRPV3 channel current.

| | TRPV3-HKE Peak Current(pA) | | | Regulating Effect% |
|---|---|---|---|---|
| Cell No. | Contro 1 | Menthol 2 mM | Menthol 2 mM+sample 0.01 g/mL | Menthol 2 mM+sample 0.01 g/mL |
| S6-211208003 | 599.65 | 4064.00 | 3635.20 | 12.38% |
| S6-211208004 | 185.9 | 4980.60 | 4126.30 | 17.82% |
| mean | 392.78 | 4522.30 | 3880.75 | 15.10% |
| SD | 292.57 | 648.13 | 347.26 | 3.85% |
| n | 2 | 2 | 2 | 2 |
| SE | 206.88 | 458.30 | 245.55 | 2.72% |

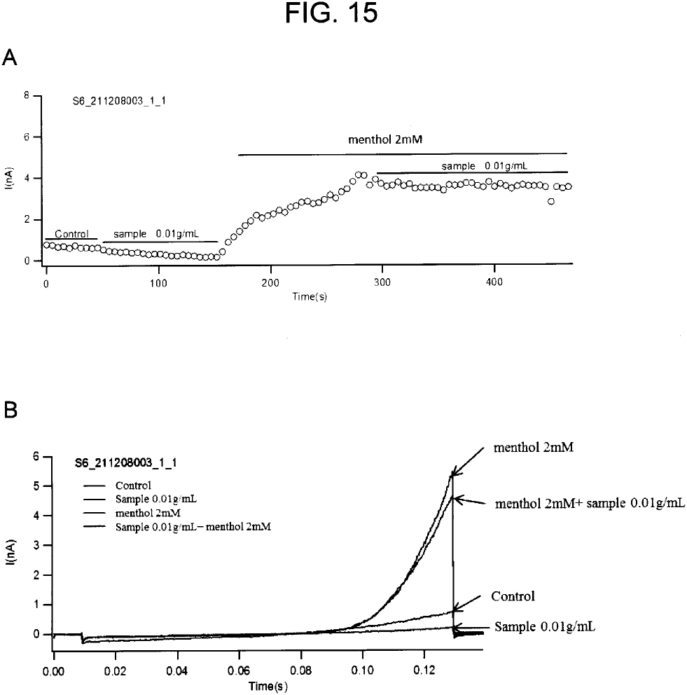

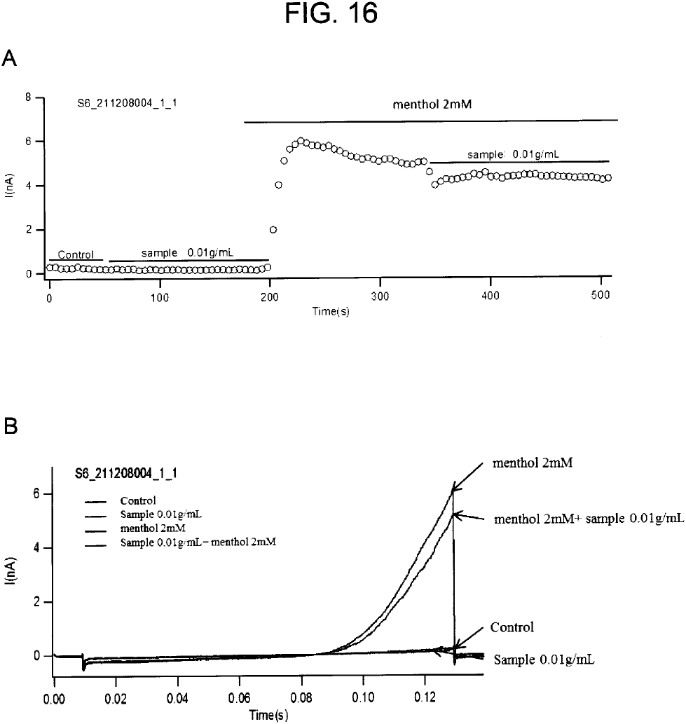

FIGS. 15 and 16 show the regulating action of different concentration GRU90-MRP-FTA (product 2-02 in Example 2) sample on TRPV3 channel current on cell S6-211208003 (FIG. 15) and cell S6-211208004 (FIG. 16).

Result evaluation

In this study, patch clamp technique was used to detect the excitability and regulating effect of GRU90-MRP-FTA (the product 2-02 in Example 2) on TRPV3 channel, so as to evaluate the influence of TRPV3 channel. The test results are summarized as follows:

Results of excitation of GRU90-MRP-FTA (the product 2-02 in Example 2) on TRPV3 current:

| Compound | % of menthol 2 mM | n |
|---|---|---|
| GRU90-MRP-FTA (the product 2-02 in Example 2) | -5.38%±3.94% | 2 |

The results showed that the GRU90-MRP-FTA (the product 2-02 in example 2) had no direct excitatory effect on TRPV3.

Results of regulating effect of GRU90-MRP-FTA (the product 2-02 in example 2) on TRPV3 current:

| Compound | Regulating Effect%-0.01 g/mL | n |
|---|---|---|
| GRU90-MRP-FTA (the product 2-02 in Example 2) | 15.10%±2.72% | 2 |

Conclusion: The results showed that the GRU90-MRP-FTA (the product 2-02 in Example 2) had no direct excitatory effect on TRPM4. However, GRU90-MRP-FTA (the product 2-02 in Example 2) had regulating effect on TRPM4. This may explain the influence of GRU90-MRP-FTA (the product 2-02 in Example 2) on cool feeling.

Note: All the Chemical reagent and Instrument are shown in the bellow tables from Example 25 to Example 27.

Chemical reagent:

| | | | |
|---|---|---|---|
| Chemical agent | brand name | Art.No. | batch number |
| DMEM | Corning | 10-013-CVR | 01521005 |
| FBS | Gibco | 10099141C | 2045512CP |
| 0.25% Trypsin-EDTA | Gibco | 25000-072 | 2323141 |
| Blasticidin | Solarbio | B9300-100 mg | 1203W021 |
| Zeocin | Solarbio | Z8020 | 714U021 |
| Lipofectamine® 3000 Transfection Reagent | Invitrogen | L3000-015 | 2332077 |
| Opti-MEM | Gibco | 51985-034 | 2193158 |
| Sodium chloride (NaCl) | Sigma | S5886 | BCCC2757 |
| Potassium chloride (KCl) | Sigma | P5405 | WXBD2577V |
| Ethylene glycol bis (2-aminoethyl ether) tetraacetic acid (EGTA) | Sigma | E3889 | SLCD5533 |
| 4- hydroxyethyl piperazine ethanesulfonic acid (HEPES) | Santa Cruz | SC-29097A | A2821 |
| Glucose (D-Glucose) | Sigma | G8270 | SLBT7079 |
| Magnesium chloride (MgCl₂•6H₂O) | Sigma | M2393 | BCCB1113 |
| Cesium chloride (CsCI) | Sigma | C4036 | 00000118328 |
| Cesium fluoride (CsF) | Innochem | A44779 | KCEG521 |
| Sodium dihydrogen phosphate (NaH₂PO₄•2H₂O) | GENERAL-REAGENT | G21298B | P1417625 |
| Phosphate buffer (PBS) | Takara | T900 | AJ1P033 |

Instrument:

| | | |
|---|---|---|
| Name | Supplier | Model |
| amplifier | Molecular Devices (USA) | Axonpatch 700B |
| Micromanipulator | Scientifica | ACCi UI |
| Electrode drawing instrument | Sutter Instruments (USA) | P97 |
| microscope | Micro-shot | MF53 |
| Capillary glass tube | Sutter Instruments (USA) | BF150-86-10 |
| Data acquisition and analysis software | HEKA (Germany) | PatchMaster & IGOR Pro |

### Example 28. Preparation of GRU90-MRP-FTAs From GRU90, Fructose and Glutamic Acid

GRU90: glycosylated rubusoside comprises unreacted rubusoside. GRU90, fructose, glutamic acid and water were weighed and dissolved in water according to Table 28-1. The solutions were then heated at about 100° C. for 1.5 hour. When the reactions were completed, the solutions were filtered through filter paper and the filtrates were dried with a spray dryer, thereby resulting in 28-01 product as an off white powder.

TABLE 28-1

Sample compositions.

| Product No. | Weight of GRU90 (g) | Weight of fructose (g) | Weight of glutamic acid (g) | Weight of water (g) |
|---|---|---|---|---|
| 28-01 | 6 | 3.696 | 0.308 | 5 |

### Example 29. Preparation of GRU90-MRP-FTAs From GRU90, Fructose, Glutamic Acid and Essential Oils/Essences

Raw material: GRU90: glycosylated rubusoside comprises unreacted rubusoside. Lemon juice aroma extract is available from Chongqing Zhengyuan flavor Co., Ltd. Lot#:Y0034434

Procedure: GRU90, fructose, glutamic acid, Lemon juice aroma extract, water were weighted as follows. The solution was then heated at about 100° C. for 2.5 hour. When the reaction was completed, the solution was filtered through filter paper and the filtrate was dried with a spray dryer, thereby resulting in product 29-01 as an off white powder.

TABLE 29-01

Test sample composition

| Product No. | Weight of GRU90 (g) | Weight of fructose (g) | Weight of glutamic acid (g) | Weight of water (mL) | Weight of Lemon Juice Aroma Extract (mL) |
|---|---|---|---|---|---|
| 29-01 | 45 | 3.75 | 1.25 | 25 | 1.2 |

### Example 30. Preparation of Mixture From GRU90-MRP-FTAs (Product of 28-01 in Example 28) and Mint Essential Oil

Material: GRU90-MRP-FTAs: product of 28-01 in Example 28.

Mint essential oil: available from ADM, Lot#: 768808002008/0406190. Procedure: GRU90-MRP-FTAs, mint essential oil, Pure propylene glycol, Pure Glycerin and water are mixed according to Table 30-1.

TABLE 30-1

mixture of GRU90-MRP-FTAs, mint essential oil etc.

| Product No. | GRU90-MRP-FTAs (product of 28-1 in Example 28) | mint essential oil | propylene glycol | water | Glycerin |
|---|---|---|---|---|---|
| 30-01 | 10000 ppm | 1000 ppm | 50 ppm | 25 ppm | 25 ppm |

### Example 31. GRU90-MRP-FTAs (Product of 28-01 in Example 28) Reduce the Salty Taste of Commercial Grapefruit Flavored Electrolyte Beverages

Commercial grapefruit flavor electrolyte beverages: grapefruit flavored Gatorade electrolyte beverages, details are shown in Table 31-1.

TABLE 31-1

Details of Grapefruit flavored Gatorade electrolyte beverages

| Product | Company | Lot# | Ingredients |
|---|---|---|---|
| Grapefruit flavored Gatorade electrolyte beverages | Pepsi Co. (China). Inc. | 20220618 | Water, sugar, edible glucose, food additives (citric acid, sodium citrate, monopotassium phosphate, food flavoring, edible salt. |

Procedure: GRU90-MRP-FTA (product of 28-01 in Example 28) was dissolved into a Gatorade electrolyte beverage with grapefruit flavored as described in Table 31-2.

TABLE 31-2

Sample compositions.

| Concentration of GRU90-MRP-FTA (ppm) | Weight of GRU90-MRP-FTA (mg) | Volume of commercial functional beverage. (mL) |
|---|---|---|
| - | - | 100 |
| 10 | 1 | 100 |
| 20 | 2 | 100 |
| 50 | 5 | 100 |

Experiment: Each sample in Table 31-2 was evaluated according to the sensory evaluation method. The results are shown in Table 31-3.

TABLE 31-3

Sensory evaluation results. The influence of different dosage of GRU90-MRP-FTA (product of 28-01 in Example 28) on the flavor of Gatorade electrolyte beverages

| | |
|---|---|
| Concentration of GRU90-MRP-FTA (ppm) | sensory evaluation |
| - | Less refreshing, salty taste |
| 10 ppm | Flavor improved, refreshing, salty taste reducing |
| 20 ppm | Flavor change a little, mouthfeel, salty taste reducing significantly |
| 50 ppm | Flavor change significantly, salty taste reducing significantly |

Conclusion: The interaction of high salty taste with the sweetness and bitterness of synthetic sweeteners makes the drink less refreshing. GRU90-MRP-FTA (product of 28-01 in Example 28) can change the salty taste. Low dose can reduce the salty taste and make the flavor more refreshing. Higher dose can significantly reduce the salty taste and improve flavor profile.

### Example 32. Product 30-01 in Example 30 Reduce the Salty Taste Of Commercial Berry Kiwi Flavored Sugar-Free Electrolyte Beverages

Commercial berry kiwi flavored sugar-free electrolyte beverages: Berry kiwi flavored sugar-free Gatorade, details are shown in Table 32-1.

TABLE 32-1

Details of berry kiwi flavored sugar-free Gatorade electrolyte beverages

| Product | Company | Lot number | Ingredients |
|---|---|---|---|
| Berry kiwi flavored sugar-free Gatorade electrolyte beverages | Pepsi Co. (China). Inc. | 20220816 | Water, food additives (citric acid, sodium citrate, food flavoring, monopotassium phosphate, sucralose, Acesulfame-K) edible salt, nicotinamide, vitamin B6. |

Procedure: Product 30-01 in Example 30 was added to the berry kiwi flavored sugar-free Gatorade electrolyte beverages at 1/1000, v/v for sensory evaluation.

TABLE 32-2

Sensory evaluation results.

| Sample | Overall likability | Salt y | Sweet lingering | Flavor | Refreshing |
|---|---|---|---|---|---|
| Base | 3 | 4 | 3 | 3 | 2 |
| Product 30-01 in Example 30 | 4 | 1.5 | 1.5 | 3.5 | 3 |

Conclusion: product 30-01 in example 3 can reduce the unpleasant salty taste and sweet lingering while improve the berry kiwi flavor, refreshing and overall likability of the beverages. The results show that product 30-01 in Example 30 can improve the berry kiwi flavored sugar-free Gatorade electrolyte beverages.

### Example 33. Product 30-01 in Example 30 Reduce the Salty Taste Of Commercial Grapefruit Flavored Electrolyte Beverages

Commercial grapefruit flavored electrolyte beverages : grapefruit flavor Gatorade electrolyte beverages: details are the same with Example 31.

Procedure: product 30-01 in Example 30 was added to the grapefruit flavored Gatorade electrolyte beverages at 1/1000 v/v for sensory evaluation.

TABLE 33-1

Sensory evaluation results.

| Sample | Overall likability | Salty | Bitterness | Flavor |
|---|---|---|---|---|
| Base | 2.5 | 4 | 2.5 | 3 |
| Product 30-01 in Example 30 | 4 | 1.5 | 1.5 | 3.5 |

Conclusion: product 30-01 in example 3 can significantly reduce the salty taste and bitterness while make the grapefruit flavor of the beverages clearer and more palatable. The results show that product 30-01 in example 3 can improve the grapefruit flavored Gatorade electrolyte beverages.

### Example 34. Product 30-01 in Example 3 Reduce the Salty Taste Of Commercial Lemon Lime Flavored Electrolyte Beverages

Commercial lemon lime flavor electrolyte beverages: lemon lime flavor Gatorade electrolyte beverages: details are shown in Table 34-1.

TABLE 34-1

Details of Lemon lime flavored Gatorade electrolyte beverages

| Product | Company | Lot number | Ingredients |
|---|---|---|---|
| Lemon lime flavor Gatorade electrolyte beverages | Pepsi Co. (China). Inc. | 20220215 | Water, sugar, edible glucose, food additives (citric acid, sodium citrate, monopotassium phosphate, food flavoring, edible salt. |

Procedure: product 30-01 in Example 30 was added to the lemon lime flavored Gatorade electrolyte beverages at 1/1000 v/v for sensory evaluation.

TABLE 34-2

Sensory evaluation results.

| | Overall likability | Salty | Bitterness | Refreshing |
|---|---|---|---|---|
| Base | 3 | 3 | 3 | 3 |
| Product 30-01 in Example 30 | 4 | 1.5 | 2 | 4 |

Conclusion: product 30-01 in Example 30 can significantly reduce the salty taste and bitterness while make the grapefruit flavor of the beverages more refreshing, clearer and more palatable. The results show that product 30-01 in Example 3 can improve the lemon lime flavor Gatorade electrolyte beverages.

### Example 35. Mixture of GRU90-MRP-FTAs (Product of 28-01 in Example 28) and GRU90-MRP-FTAs (Product of 29-01 in Example 29) Reduce the Salty Taste of Commercial Lemon Lime Flavored Electrolyte Beverages

Commercial lemon lime flavor electrolyte beverages: Lemon lime flavored Gatorade electrolyte beverages: details are the same with Example 34.

GRU90-MRP-FTAs: product of 28-01 in Example 28, GRU90-MRP-FTAs: product of 29-01 in Example 29.

Process: GRU90-MRP-FTA (product of 28-01 in Example 28) and GRU90-MRP-FTAs (product of 29-01 in Example 29) were dissolved into a Lemon lime flavored Gatorade electrolyte beverages as described in Table 35-1.

TABLE 35-1

Sample compositions.

| Product No. | Weight of GRU90-MRP-FTA (product of 28-01 in Example 28) (mg) | Weight of GRU90-MRP-FTA(product of 29-01 in Example 29) (mg) | Volume of commercial functional beverage. (mL) |
|---|---|---|---|
| Base | - | - | 100 |
| 35-01 | 10 | 10 | 100 |

Experiment: each sample in Table 35-1 was evaluated according to the sensory evaluation method. The results are shown in Table 35-2.

TABLE 35-2

Sensory evaluation results.

| | Overall likability | Salt y | Bitterness | Refreshing |
|---|---|---|---|---|
| Base | 3 | 3 | 3 | 3 |
| 35-01 | 4 | 1.5 | 1.5 | 4.5 |

Conclusion: mixture of GRU90-MRP-FTAs (product of 28-01 in Example 28) and GRU90-MRP-FTAs (product of 29-01 in Example 29) can significantly reduce the salty taste and bitterness while make the lemon lime flavor of the beverages more refreshing, clearer and more palatable. The results show that mixture of GRU90-MRP-FTAs (product of 28-01 in Example 28) and GRU90-MRP-FTAs (product of 29-01 in Example 29) can improve the lemon lime flavored Gatorade electrolyte beverages.

### Example 36. Number of Positions Available for Glucosylation (n=1-10) of GRA100

Rebaudioside A offers 3 practical positions (see arrows in Formula III below) for (α 1 →4) extensions with glucose using a glucose donor and glucosyltransferases. A fourth position (see broken arrow in Formula III) is theoretically available only as it would create branching and not extension and is sterically hindered. Considering that glucosyltransferases do not act site-specific, all three available binding positions will be glucosylated in a random order. Subsequent glucosylations are also expected to appear in random order whereby the total number of extension positions remains 3, irrespectively of the number of glucose units attached.

Mathematically, glucosylation follows then the exponential function V = nm where V denotes all possible structural variants, m denotes the number of glucose units attached and n denotes the number of possible positions for glucosylation.

The number of all possible structural variants includes molecules which are chemically identical, but the sequence of glucosylation to yield the identical structure is different. The number of variants with different chemical structures (structural isomers) follow the formula I = (n + m - 1)! / ((n - 1)! * m!) where I denotes structural isomers, m denotes the number of glucose units attached and n denotes the number of possible positions for glucosylation.

Exemplifying, the possible sequence of adding 2 or 3 glucose units is shown below on Table 36-1. The 3 sites for the first glucose unit are denoted as a1, b1, c1 (step 1) the variations for the second glucose unit are denoted as a2, b2 and c2 (step 2) or a3, b3, c3 (step3). Variants in step 2 or 3 with identical chemical structure but different sequence are shown in one row.

Table 36-2 summarizes the number of all possible structural variants and structural isomers for various numbers of initially available (α 1→4) positions for glucosylation. Mathematically the difference between possible structure and structural isomers is explained by considering versus neglecting the sequence of glucosylation.

The formula below shows positions available for (α 1-4) glucosidic bonds in Rebaudioside A (marked by arrows)

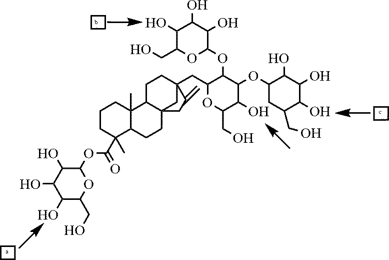

Table 36-1, Structural variants for glucosylation with 2 or 3 glucose in (α 1→4) positions. Each panel shows all possible variants and each row represents one structural isomer.

Structural variants for glucosylation with 2 glucose in (α 1→4) positions.

| | |
|---|---|
| a1a2 | |
| a1b2 | b1a2 |
| a1c2 | c1a2 |
| b1b2 | |
| b1c2 | c1b2 |
| c1c2 | |

Structural variants for glucosylation with 3 glucose in (α 1→4) positions.

| | | | | | |
|---|---|---|---|---|---|
| a1a2a3 | | | | | |
| b1a2a3 | a1b2a3 | a1a2b3 | | | |
| c1a2a3 | a1c2a3 | a1a2c3 | | | |
| a1b2c3 | a1c2b3 | b1a2c3 | b1c2a3 | c1a2b3 | clb2a3 |
| b1b2b3 | | | | | |
| a1b2b3 | b1a2b3 | b1b2a3 | | | |
| c1b2b3 | b1c2b3 | b1b2c3 | | | |
| c1c2c3 | | | | | |
| a1c2c3 | c1a3c3 | c1c2a3 | | | |
| b1c2c3 | c1b2c3 | c1c2b3 | | | |

Legend: a, b, c denotes the positions available for (α 1→4) glucosylation; 1, 2, 3 denotes the sequence of glucosylation.

TABLE 36-2

Generalized Overview on possible variants (pv) and structural isomers (is) dependent on the number of available positions for glucosylation and number of glucose units attached.

| number of glc units attached (m) | number of positions available for glucosylation (n) | | | | | | | | | |
|---|---|---|---|---|---|---|---|---|---|---|
| | 1 | | 2 | | 3 | | 4 | | 5 | |
| | pv | si | pv | si | pv | si | pv | si | pv | si |
| 1 | 1 | 1 | 2 | 2 | 3 | 3 | 4 | 4 | 5 | 5 |
| 2 | 1 | 1 | 4 | 3 | 9 | 6 | 16 | 10 | 25 | 15 |
| 3 | 1 | 1 | 8 | 4 | 27 | 10 | 64 | 20 | 125 | 35 |
| 4 | 1 | 1 | 16 | 5 | 81 | 15 | 256 | 35 | 625 | 70 |
| 5 | 1 | 1 | 32 | 6 | 243 | 21 | 1024 | 56 | 3125 | 126 |
| 6 | 1 | 1 | 64 | 7 | 729 | 28 | 4096 | 84 | 15625 | 210 |
| 7 | 1 | 1 | 128 | 8 | 2187 | 36 | 16384 | 120 | 78125 | 330 |
| 8 | 1 | 1 | 256 | 9 | 6561 | 45 | 65536 | 165 | 390625 | 495 |
| 9 | 1 | 1 | 512 | 10 | 19683 | 55 | 262144 | 220 | 1953125 | 715 |
| 10 | 1 | 1 | 1024 | 11 | 59049 | 66 | 1048576 | 286 | 9765625 | 1001 |

An embodiment of sweetener or flavor composition of the present application comprises one or more glycosylated stevia glycosides as per Formula III and/or Table 36-2. Additionally, the structural variants for glucosylation described in Formula III and Table 36-2 also applies to other stevia glycosides, wherein the structure of Rebaudioside A is replaced by stevioside, rubusoside, rebaudioside B, Rebaudioside C, Rebaudioside D, Rebaudioside M etc.

The above description is for the purpose of teaching a person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A composition comprising an agent that regulates a cooling effect or taste, wherein the agent comprises one or more nature high-intensity sweeteners (NHIS) and/or derivatives of NHIS, and wherein the agent is present in the composition in a sufficient amount to regulate a cooling effect or taste.

2. The composition of claim 1, wherein the one or more NHIS and the one or more derivatives of NHIS are selected from the group consisting of stevia extracts (SEs), glycosylated stevia extracts (G-SEs), stevia extract-MRPs (SE-MRPs), glycosylated stevia extract-MRPs (G-SE-MRPs), stevia glycosides (SGs), glycosylated stevia glycosides (GSGs), stevia glycoside-MRPs (SG-MRPs), glycosylated stevia glycoside-MRPs (GSG-MRPs), sweet tea extracts (STEs), glycosylated sweet tea extracts (G-STEs), sweet tea extract-MRPs (ST-MRPs), glycosylated sweet tea extract-MRPs (G-ST-MRPs), sweet tea glycosides (STGs), glycosylated sweet tea glycosides (G-STGs), sweet tea glycoside-MRPs (STG-MRPs), glycosylated sweet tea glycoside-MRPs (G-STG-MRPs), monk fruit extracts (MFEs), glycosylated monk fruit extracts (G-MFEs), monk fruit glycosides (MFGs), glycosylated monk fruit glycosides (G-MFG), monk fruit glycoside-MRPs (MFG-MRPs) and glycosylated monk fruit glycoside-MRPs (G-MFG-MRPs).

3. The composition of claim 1, wherein the agent comprises a glycosylated stevia glycoside-MRP (GSG-MRP).

4. The composition of claim 3, wherein the GSG-MRP comprises unreacted GSG, unreacted SG, unreacted amine donor and/or unreacted sugar donor.

5. The composition of claim 1, further comprising one or more cooling agents selected from the group consisting of icilin, eucalyptol, camphor, eucalyptol, linalool, geraniol, hydroxycitronellal, menthoxypropanediol (cooling agent 10), CPS-113, CPS-369, Frescolat ML and Frescolat MGA, menthol, menthone, menthol esters, ethers and menthane carboxamides.

6. The composition of claim 1, wherein the composition further comprises at least one flavoring substance.

7. The composition of claim 1, wherein the agent is present in an amount in the range of 0.001-99 wt% of the composition.

8. A consumable product comprising the composition of claim 1.

9. The consumable product of claim 9, further comprising one or more warming, bitter or tingling substances.

10. A method of improving a taste profile of a consumable product, comprising adding to the consumable product, a sufficient amount of the composition of claim 1, wherein the coolness of the consumable product is improved after the addition of the composition.

11. The method of claim 10, wherein the consumable product is a food product, a beverage, a candy, a chewing gum or an oral hygiene product.

12. A method for improving a cooling profile in a composition, comprising adding one or more nature high-intensity sweeteners (NHIS) and/or one or more derivatives of NHIS to the composition, wherein the one or more nature high-intensity sweeteners (NHIS) and/or one or more derivatives of NHIS improves coolness of the composition.

13. The method of claim 12, wherein the one or more NHIS and the one or more derivatives of NHIS are selected from the group consisting of stevia extracts (SEs), glycosylated stevia extracts (G-SEs), stevia extract-MRPs (SE-MRPs), glycosylated stevia extract-MRPs (G-SE-MRPs), stevia glycosides (SGs), glycosylated stevia glycosides (GSGs), stevia glycoside-MRPs (SG-MRPs), glycosylated stevia glycoside-MRPs (GSG-MRPs), sweet tea extracts (STEs), glycosylated sweet tea extracts (G-STEs), sweet tea extract-MRPs (ST-MRPs), glycosylated sweet tea extract-MRPs (G-ST-MRPs), sweet tea glycosides (STGs), glycosylated sweet tea glycosides (G-STGs), sweet tea glycoside-MRPs (STG-MRPs), glycosylated sweet tea glycoside-MRPs (G-STG-MRPs), monk fruit extracts (MFEs), glycosylated monk fruit extracts (G-MFEs), monk fruit glycosides (MFGs), glycosylated monk fruit glycosides (G-MFG), monk fruit glycoside-MRPs (MFG-MRPs) and glycosylated monk fruit glycoside-MRPs (G-MFG-MRPs).

14. The method of claim 13, wherein the one or more derivatives of NHIS comprises a glycosylated stevia glycoside-MRP (GSG-MRP).

15. The method of claim 14, wherein the GSG-MRP comprises unreacted GSG, unreacted SG, unreacted amine donor and/or unreacted sugar donor.

16. The method of claim 12, wherein the composition comprises one or more cooling agents selected from the group consisting of icilin, eucalyptol, camphor, eucalyptol, linalool, geraniol, hydroxycitronellal, menthoxypropanediol (cooling agent 10), CPS-113, CPS-369, Frescolat ML and Frescolat MGA, menthol, menthone, menthol esters, ethers and menthane carboxamides.

17. A method to regulate a salty taste of a salt-containg consumable, comprising:
adding to the salt-containing consumable a composition comprising a Maillard reaction product,
wherein the Maillard reaction product is prepared from a reaction mixture that comprise a glycosylated steviol glycoside, and
wherein the Maillard reaction product is added at a final concentration of 1 ppm to 0,000 ppm.

18. The method of claim 17, wherein the glycosylated steviol glycoside is glycosylated rubusoside.

19. A flavor or sweetening composition, comprising:
one or more high intensity sweeteners; and
glycerin,
wherein the weight ratio of glycerin-to-high intensity sweeteners is in the range of 99:1 to 1:99.

20. A food or beverage, comprising the flavor or sweetening composition of claim 19.
